SEQUENCE LISTING (69 sequences)

SEQ ID NO: 1 - protein, 170 aa, Taraxacum officinale
MADNGNTGRQKDDDGGHDGPRQNPTTPPSPSRTPRRPRRNTSPPKHSPGASSSTMPAPPTPPAPTGITGASSSSVGTNIISFIPPKTKRTKSVICPICNKDMCHEKALCGHIRWHTQEERLAASIAIARALSSNVVVSGNGDEDEGPSKKYKLPDLNKSPPPEEEDEDAA

SEQ ID NO: 2 - DNA, 1900 bp, Taraxacum officinale
  source 1..1900: mol_type = unassigned DNA
taaggccataacaaggttatttgaacattcgcctaaaccctaaaccaactatatctattcggatttgttacgagttttagagtttctagaaaatcagttatatatcgattaggcttggttctaggttataagaaaaaatgattctctatccggtatgacatagagacaaaccggtctacttttataagaaaatcatatttgaaacaatgtctagttaataattaattaatatgtgcatgtttaaatttagaatatcctgaatttatatttagtgcgcaaccctatgtaagatcgaactaattttaaaccgtaaccagacatgcatgtttgatcagtaataaaaaggctattatcgtcgaaaatattaaaacaaaatataatatcttatttggcgccatttcatgtcaacggttgaaagcaagttaccaacgcctccaaccgatcatttctccggtaacaaccaactgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctactaataaccgccaccgcaaaaacctataaatacaacttccatcacaattctgtcccctactcccatttcaatccaaaaaaaggctacacagacatatacagcg

SEQ ID NO: 3 - DNA, 513 bp, Taraxacum officinale
  source 1..513: mol_type = unassigned DNA
atggcagataatggcaacaccggccgtcaaaaggatgacgacggtggccatgatggaccacgccaaaacccaactactccaccctccccttcccgcacccctcgaagaccaaggcggaacacatcaccgcccaaacattctccgggggcgtcttcaagcaccatgccagcgccgcctactccccctgcgccgacgggaatcaccggtgcttctagttcttctgtgggtactaatataatttcatttattccacccaaaaccaaaagaacgaagtcggtgatctgcccgatctgcaacaaagatatgtgccatgagaaggcgctgtgtggccacatccggtggcatacacaggaggaaagattggcggccagcatcgctatagcaagagcgctatcttctaacgttgttgtttctggcaatggcgatgaagatgaaggtccatctaaaaagtataaactcccggacctgaacaaatctccaccgccggaggaggaggacgaggacgctgcctga

SEQ ID NO: 4 - DNA, 5356 bp, Taraxacum officinale
  source 1..5356: mol_type = unassigned DNA
ttttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcatgatattgcaggaatgtaacttagaactcaacgataaaaagtgtgttgagatcgtggaagttgatcatttatcttgaattcataagataagcaacatgagataggataattaattactgtggggatttctttttatattcatattattcgttgtaatgcaaatttttaagtagaataacgaatgtgtcttattatttacggttaaatgcaaaagatcattaacatatctccattgatttgtttactgatgtattctcatgtgttctgttgctaaattctattttgaaaagtatattcaattaaagcaatgcattttagatataaagcaactccataacttataattgtgtagatttttaccaattcaatgtcttaaaagaaaaataaaaattgaaagtttaattaattgccatagtgatcccgttcccgtcaattatgtgatcaatttttaccagagcaaaaccccacatttcttgtagtgaacaatctgtggatgataaccgatcgaagacttttgtggggatttcactaaccaactatttggctctcattaattactattagttatataaggccatcccaagtattttggcaatattcaaaacgagtaaactataaaattacacttttggttcttgtatttctaacttttagaattatgtatttggttcttatagttttttatttgtatgaattacattacttttggtccacaatattttttattttctataattccatttttagtcattgtagttttaatttgcaataaaaaattaatttatatttttaatccatatatatagttggcaatattctatattttaggttcagattgtatcttttataaaaaagagtcttaaaagactaaaccttttggatccttatagttttcacaatcacttgtgatttttttaatgaacatatcatatttcttaaattacatcagtaatctacgtataacatttaatataattttatttaatcagaaatataggattcgtataataactttgctgaacttaggcttcatttttaacaaaggataatgcatcatgcatgacataaaaatttgtaatatttctaatgtgcatgcactacaagaacctgacccttcatatacattaatgaatgattctcttttcttatttaatcttagcaagcggtttggataattgtctgcacaatattaagaattgcactagctagctaggggagtccatgacgtaataagcaaattaactagaggacttgtaggaggcttgatcacttcattgtatatctaatggcactatcgatatatgattggcttgtgtgtatctatggaatttaacactattaaaaaatatatctttaatgacaatcaacaacacacagtaatttatgacttacaaaatcatgtgtgattaaaaaaaatgtcatgttttttaaaattatagaggatttacgacttacataattatgtgtcataattatatgtcatcttgtgtaatatttatgacttacaaaaatgtatgtcatgttttatcatcataaaatctataaaaaaaaatagaaacacgaggatactttcagtgtatgttatctgtaaaaaaaaataaaaactcggaggacactaaaagtgtagaaacttgacagcataaacgaatttatttaccttcatcaaaacttattttacttcaatctctgaaccagctcccttcattaatctcttcgagatgtaggttgattttaatgctaaatgaacttttcaaatgtataaataaaacttttctgtgccagatttgacattgatggagatgaagatacacaagtgaattcacctttcaatgatcatactgacataccaagtactcctatcccctctccacctccacaaaacgaacctcaatttcaaaaacgtaactatcatttaaatatttaatcatttttcaattggattcatgcggtaatagaagttactggactttaaactttttgtagctgatgagattcatgagcaacagtccatggacattttccaaatcaacaagtttctgatgaggacaacgtaaaaaaagtttttcaattattaaacatatgattttggtttttaaaagataattaattatggaaaatcttcagaaaagtctcaatactttgggtttattaatgaaaaagtctcaaaaaaaaatttgaataaaaaagtccataaaattggataaattattcgatttagcccaatttgccaagtaaatctggtaaaattaggctaaatcgaataatttagccagtttggtgggcctttttgcgcaaaaacattattttgggacttaatcgttaattttccccaaatttcagtttatttacagtcttggagtaaaaccgaacctagtgcgctaattttggcttttggtgtttggaattatttgggtttattgggctcggtttctgaacgagtttgatttttggtttcttgattcaattttttggttttggggttgaacccatggattttcgatttggtttcggctttgataatatttttgtgcaatcgttcaaaatatccgagttagattgggtttaaatgttatcaaaaccgaaaccgagggtagaaccccaaaactgaacccaatcgaaaatccataggttgaaccccagaaccgaacccaataaactctaaaaaccaaaagccaaaattaaccgataaaaaacttaaaatacatgtttgttaaatctagtaaggtgtttttgacactttatgtaaattatagaaaaaaatggattaactgtttgcttattaagataaaaacgacgtaaaaatagaattggattagaaatgaaccatttttttacatcgtttactttatttactaggttataacccgtgtattacacgggttgacgataaaaaattgtataaaaaaacaacatataatggtaagtttgtaatcgtacatgggttgcttcaattttaatttaaaaattgtaattccaaatgaaatatatgtaggatgatataacatacttgtaaatgtaaatgagagtttgttggcatttcatacatttagcccatcaaatcaaacgcaaaaaatctttttaatcctataatacatgtggcacaaatatacgattagcatttcgctgagagagaggctttaagtgaggatgtgaaaaagaaggaacatagcaacttggaatttaaagatcaaaaaaccttgaaaggcttttggaatgtaaaaatatagaaatgaataaatgtggcttcttgtatgtatcaagagtaggacctgtattagaaacataaaaaatttgtgtctaataaaaatcaacaaaaacagaacgtaaacgaaagaagttattggtaaaaaaagatttcaaaaatagtttttttttgaaccggttcaaaaatagttattttacttatttatccatttaatccaaacttttttttgtcaaatatctcatggaattagttggctgcgtcactcttgtcactgggcgttataacttttgccttgtggtttgcaacaccaccttaaataacatagacaaataacattaaatatcatgataatacataataactcacaaaagatgcattctatagaacggtataattgattagaacaaaataatctaaatagtttatgaggattaccttgataattcccttggagtgaatactatccacgatgtcatcttccaaaggtattcaatggcttttattagaacccaatttcagaattacattgatgccacagttacattcactaaatatcttcctcttaggcttttagtttgactatattttaaccattttcacatcacactttaaccaccccctttggttattaatgtcaaaaggaacaaaactcacaaatcaaatgcgtggtgtttatactattggtacaatgtttataaatttcttaaaaatgagcacaatttcaaaaaacttaaacaaatgtcaatggtcaccatgtggtattaatattataaagttctaaaaataccattccaccccaacatggtactaaccagttagaaaatcaagaaacagaaacatactctttaatctaaaacaatataatatagggatgtgttatgagtttagtttcatctaaccatgaattcatataggattgtacgtttctcctcgaaattgcaaagaattttgttcgactaagcaatctacatattaatggatttgtatggtttctcggtttgtaggtaagttaagagctaccaggaaagagaaaatttgcaccataggtactaaacacattctacaatggtggctttaacgtgagagagaatagagaggtcgaaacatatttccgttaaataagcaaacataagtttctatttgggatcaataagtttcttcctatagtggtaaatggatctgtacttgttaagctcttaaacatatttgttgtcactgttcacttcctattttgcagtgctcttctattacttacattcttcttatgacattcttttaataaggaaaaataaccacttattctagtattctattgttttgttagagtcaatatcagataagaacctaaaaaggtgggacaaattgataacttaatcaatatatcaaactgtcatatgtctcaagtctcaacccaaattgatatgtttaagaaaaattcagaaaagatatgtccatgggacatgtgcatgtatatgtataagtgtacattcctaaaaaaagtaggctcatatgacatgagcatgtatgtgcttattgttataaagattacaaattttcttaaaaatgaacacaatttcaaaatacctataccaaagcccatgatcaacatagcatctgatattaatattataaagctctaaacaaatctaccccgacatcaagtggtacttacaagttggaaaatcaagatacaaaaacatagcttttaatctaagtaaaaacacaataatatagggatatgacctatatgcatgtttaaaatcttaagtctgtaaagattgataggattggaaaactcttctattcaacacaagaaaagaattacagtaggaggagggactctcacactcaactactacaaaaaaaccaaccctcacttactaacactctcacactagtg

SEQ ID NO: 5 - DNA, 7769 bp, Taraxacum officinale
  source 1..7769: mol_type = unassigned DNA
taaggccataacaaggttatttgaacattcgcctaaaccctaaaccaactatatctattcggatttgttacgagttttagagtttctagaaaatcagttatatatcgattaggcttggttctaggttataagaaaaaatgattctctatccggtatgacatagagacaaaccggtctacttttataagaaaatcatatttgaaacaatgtctagttaataattaattaatatgtgcatgtttaaatttagaatatcctgaatttatatttagtgcgcaaccctatgtaagatcgaactaattttaaaccgtaaccagacatgcatgtttgatcagtaataaaaaggctattatcgtcgaaaatattaaaacaaaatataatatcttatttggcgccatttcatgtcaacggttgaaagcaagttaccaacgcctccaaccgatcatttctccggtaacaaccaactgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctactaataaccgccaccgcaaaaacctataaatacaacttccatcacaattctgtcccctactcccatttcaatccaaaaaaaggctacacagacatatacagcgatggcagataatggcaacaccggccgtcaaaaggatgacgacggtggccatgatggaccacgccaaaacccaactactccaccctccccttcccgcacccctcgaagaccaaggcggaacacatcaccgcccaaacattctccgggggcgtcttcaagcaccatgccagcgccgcctactccccctgcgccgacgggaatcaccggtgcttctagttcttctgtgggtactaatataatttcatttattccacccaaaaccaaaagaacgaagtcggtgatctgcccgatctgcaacaaagatatgtgccatgagaaggcgctgtgtggccacatccggtggcatacacaggaggaaagattggcggccagcatcgctatagcaagagcgctatcttctaacgttgttgtttctggcaatggcgatgaagatgaaggtccatctaaaaagtataaactcccggacctgaacaaatctccaccgccggaggaggaggacgaggacgctgcctgattttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcatgatattgcaggaatgtaacttagaactcaacgataaaaagtgtgttgagatcgtggaagttgatcatttatcttgaattcataagataagcaacatgagataggataattaattactgtggggatttctttttatattcatattattcgttgtaatgcaaatttttaagtagaataacgaatgtgtcttattatttacggttaaatgcaaaagatcattaacatatctccattgatttgtttactgatgtattctcatgtgttctgttgctaaattctattttgaaaagtatattcaattaaagcaatgcattttagatataaagcaactccataacttataattgtgtagatttttaccaattcaatgtcttaaaagaaaaataaaaattgaaagtttaattaattgccatagtgatcccgttcccgtcaattatgtgatcaatttttaccagagcaaaaccccacatttcttgtagtgaacaatctgtggatgataaccgatcgaagacttttgtggggatttcactaaccaactatttggctctcattaattactattagttatataaggccatcccaagtattttggcaatattcaaaacgagtaaactataaaattacacttttggttcttgtatttctaacttttagaattatgtatttggttcttatagttttttatttgtatgaattacattacttttggtccacaatattttttattttctataattccatttttagtcattgtagttttaatttgcaataaaaaattaatttatatttttaatccatatatatagttggcaatattctatattttaggttcagattgtatcttttataaaaaagagtcttaaaagactaaaccttttggatccttatagttttcacaatcacttgtgatttttttaatgaacatatcatatttcttaaattacatcagtaatctacgtataacatttaatataattttatttaatcagaaatataggattcgtataataactttgctgaacttaggcttcatttttaacaaaggataatgcatcatgcatgacataaaaatttgtaatatttctaatgtgcatgcactacaagaacctgacccttcatatacattaatgaatgattctcttttcttatttaatcttagcaagcggtttggataattgtctgcacaatattaagaattgcactagctagctaggggagtccatgacgtaataagcaaattaactagaggacttgtaggaggcttgatcacttcattgtatatctaatggcactatcgatatatgattggcttgtgtgtatctatggaatttaacactattaaaaaatatatctttaatgacaatcaacaacacacagtaatttatgacttacaaaatcatgtgtgattaaaaaaaatgtcatgttttttaaaattatagaggatttacgacttacataattatgtgtcataattatatgtcatcttgtgtaatatttatgacttacaaaaatgtatgtcatgttttatcatcataaaatctataaaaaaaaatagaaacacgaggatactttcagtgtatgttatctgtaaaaaaaaataaaaactcggaggacactaaaagtgtagaaacttgacagcataaacgaatttatttaccttcatcaaaacttattttacttcaatctctgaaccagctcccttcattaatctcttcgagatgtaggttgattttaatgctaaatgaacttttcaaatgtataaataaaacttttctgtgccagatttgacattgatggagatgaagatacacaagtgaattcacctttcaatgatcatactgacataccaagtactcctatcccctctccacctccacaaaacgaacctcaatttcaaaaacgtaactatcatttaaatatttaatcatttttcaattggattcatgcggtaatagaagttactggactttaaactttttgtagctgatgagattcatgagcaacagtccatggacattttccaaatcaacaagtttctgatgaggacaacgtaaaaaaagtttttcaattattaaacatatgattttggtttttaaaagataattaattatggaaaatcttcagaaaagtctcaatactttgggtttattaatgaaaaagtctcaaaaaaaaatttgaataaaaaagtccataaaattggataaattattcgatttagcccaatttgccaagtaaatctggtaaaattaggctaaatcgaataatttagccagtttggtgggcctttttgcgcaaaaacattattttgggacttaatcgttaattttccccaaatttcagtttatttacagtcttggagtaaaaccgaacctagtgcgctaattttggcttttggtgtttggaattatttgggtttattgggctcggtttctgaacgagtttgatttttggtttcttgattcaattttttggttttggggttgaacccatggattttcgatttggtttcggctttgataatatttttgtgcaatcgttcaaaatatccgagttagattgggtttaaatgttatcaaaaccgaaaccgagggtagaaccccaaaactgaacccaatcgaaaatccataggttgaaccccagaaccgaacccaataaactctaaaaaccaaaagccaaaattaaccgataaaaaacttaaaatacatgtttgttaaatctagtaaggtgtttttgacactttatgtaaattatagaaaaaaatggattaactgtttgcttattaagataaaaacgacgtaaaaatagaattggattagaaatgaaccatttttttacatcgtttactttatttactaggttataacccgtgtattacacgggttgacgataaaaaattgtataaaaaaacaacatataatggtaagtttgtaatcgtacatgggttgcttcaattttaatttaaaaattgtaattccaaatgaaatatatgtaggatgatataacatacttgtaaatgtaaatgagagtttgttggcatttcatacatttagcccatcaaatcaaacgcaaaaaatctttttaatcctataatacatgtggcacaaatatacgattagcatttcgctgagagagaggctttaagtgaggatgtgaaaaagaaggaacatagcaacttggaatttaaagatcaaaaaaccttgaaaggcttttggaatgtaaaaatatagaaatgaataaatgtggcttcttgtatgtatcaagagtaggacctgtattagaaacataaaaaatttgtgtctaataaaaatcaacaaaaacagaacgtaaacgaaagaagttattggtaaaaaaagatttcaaaaatagtttttttttgaaccggttcaaaaatagttattttacttatttatccatttaatccaaacttttttttgtcaaatatctcatggaattagttggctgcgtcactcttgtcactgggcgttataacttttgccttgtggtttgcaacaccaccttaaataacatagacaaataacattaaatatcatgataatacataataactcacaaaagatgcattctatagaacggtataattgattagaacaaaataatctaaatagtttatgaggattaccttgataattcccttggagtgaatactatccacgatgtcatcttccaaaggtattcaatggcttttattagaacccaatttcagaattacattgatgccacagttacattcactaaatatcttcctcttaggcttttagtttgactatattttaaccattttcacatcacactttaaccaccccctttggttattaatgtcaaaaggaacaaaactcacaaatcaaatgcgtggtgtttatactattggtacaatgtttataaatttcttaaaaatgagcacaatttcaaaaaacttaaacaaatgtcaatggtcaccatgtggtattaatattataaagttctaaaaataccattccaccccaacatggtactaaccagttagaaaatcaagaaacagaaacatactctttaatctaaaacaatataatatagggatgtgttatgagtttagtttcatctaaccatgaattcatataggattgtacgtttctcctcgaaattgcaaagaattttgttcgactaagcaatctacatattaatggatttgtatggtttctcggtttgtaggtaagttaagagctaccaggaaagagaaaatttgcaccataggtactaaacacattctacaatggtggctttaacgtgagagagaatagagaggtcgaaacatatttccgttaaataagcaaacataagtttctatttgggatcaataagtttcttcctatagtggtaaatggatctgtacttgttaagctcttaaacatatttgttgtcactgttcacttcctattttgcagtgctcttctattacttacattcttcttatgacattcttttaataaggaaaaataaccacttattctagtattctattgttttgttagagtcaatatcagataagaacctaaaaaggtgggacaaattgataacttaatcaatatatcaaactgtcatatgtctcaagtctcaacccaaattgatatgtttaagaaaaattcagaaaagatatgtccatgggacatgtgcatgtatatgtataagtgtacattcctaaaaaaagtaggctcatatgacatgagcatgtatgtgcttattgttataaagattacaaattttcttaaaaatgaacacaatttcaaaatacctataccaaagcccatgatcaacatagcatctgatattaatattataaagctctaaacaaatctaccccgacatcaagtggtacttacaagttggaaaatcaagatacaaaaacatagcttttaatctaagtaaaaacacaataatatagggatatgacctatatgcatgtttaaaatcttaagtctgtaaagattgataggattggaaaactcttctattcaacacaagaaaagaattacagtaggaggagggactctcacactcaactactacaaaaaaaccaaccctcacttactaacactctcacactagtg

SEQ ID NO: 6 - protein, 227 aa, Taraxacum officinale
MSTVKSKLSTPPTDHFSGNHQLLLITATAKTYIYNFHHNSVPYSHFNPKKATQTYTAMADNGNTGRQKDDDGGHDGGRPNPTTPPSPSRTPRRPRRTTSPPKHSPGASSSTMPAPPTPPAPTGITGASSSSVGTNIISFTPPKTKRTKSVICPICKKDMCHEKALCGHIRWHTQEERLAASIAIARALSSNVVVSGNGDEDEGPSKKYKLPDLNKSPPPEEEDEDAA

SEQ ID NO: 7 - DNA, 14519 bp, Taraxacum officinale
  source 1..14519: mol_type = unassigned DNA
ctgacttggccaacattttttgttcttccgatcccttcgtaacaatcagtttaagcacaattgaattatatggagataaataggtgaaatttggaagacgaggatgggagagtgcgaagagccaaagaagagaaatgtgggatagcggccggccggttgttaatagaagtgcttagatcttggagaccttctaatgcttatggaggaaccagatgcttcaatttgtaaatctgcgctgcccacgtagaatattgaaaaagaaaatcaaatcgatcaataatgcgaccatatactttaggatttcccacttgtataattctcttattcattttcaaatctttatctaaattaaggatatcggcaaaaatataatcctaattgtttttcaaaatgataaggattataaggtaacttaattcttgttttattcatatttaattattattgttttttatcaatgttttaaaacccgggttttgagtcaacccggtcttgtgaaaaatcccgggtcagccggtcagctgatctaccggttcaatatatttaaataaaatataaattttcatatagaatttgagttttaggctaagaaaatcgggttttgggctaaggaaaatcgggtttaagttttgggctaatggattttcgggtcaataacgtttttttccgttttttagatcggttcgaccgcgttttgtgtgaaacccggccgggttgatccgagtcaataattggcaacaacccggtatatgttgacccgctcttgtccccgggtcccggttcaaccggttggatcggccgggttgaccctggttttaaaacactgttttttatacaatatatttgtttttgtttttagttttgtttttgtttttgattttgtagatatatttttaattttatttttattttgtataaatatagtcattacaaccgattatttgtaagttgagtcggtcaaaacgaactttctgataattcttatacatttttcggcgaaacatgaatattttgatcaaccaatatgcttttttctaggttttttaaataaaccgcacgttcaattgcatattcgcaaacaatagaactcgaaaaccctaatcgagcttgttttatagaaatctgggttgagtgcatacataataattaataactgtgaaaaagtttgttttaacatgctaaacctatcgatagtcggttataataattatatgtaaacaaaatcagaaacaataatgatatatgtcacccaaaaaaatagttactaaatatatacaaaatcaaaaacaacagtaatttcttttacaaaaaaaagagaacaatagtgactaactttagaaagaaaaaaaaagaactaaattactcttataaatcattatcttttatataaattggtaaattggtcatgtaatctagaatgtgaattgcggaatgctttagtcacattttgaacaaactggtaattattaatttcgataaaagttcttcaagaatagtttggtcacattttggacgacaatggtgtgttttccatagtaagcaatacgtaatgagttcgcttaataggaaaatagatctaaagcttaatgaaatcatatgtataagtaacatatatttttcacacgttacattgtttgtaacgtcctcaatttttttaatatatataactaggtacactctaaggtcgtgctaagcacgacccacgaatggtaatttaggtaatttttcatccttgactaaaatacattatgctaataacaaagaattaagaatacaatgctataatgcataagtaaataaaagtacatgatatttctcacatttagtctttatacatagaaaacttacactggccatttgcaaaaaaacattaaatctccctaaaaagtagagtttacaaaatgaaagcttgtttttatagcaaacaataatcaatatgtgaagctatagtatagtaacctactcatttcaacttttaggcagtgcagtaattacttactcatgttaatggtgaaatgcattaagataaacttcattcaacggattaaatctgggttcattataaatgtactcttagttactcctccatgtaactgcatttagcgaacaacaaaagctctaataaataatatgaaacacatgctaatttataaatgacatcatgggtatagatgctgtaacatcctaaacttttaaaataaataataaatcatatttgggatttaaagtaatattttaagataattgataaataagagaatgaaatacaaagaggcattttatttttagcctttgatttataaaaaagaaaaaaccttgaaattggatgtattttgaaatgggtaagtattttaaaagatgtgtaagttaagggggataaatggaaattaaggagataagcatttgaccaaagtcaaaatagaaggaacaataagtcggtcgattgttttcctgtgtgcatcttcggacgacagagagacagaaaagaacgagcgagagacagagaggaatcgagacagagagattatcgagagagagacagcgaagtcgggagagggagaccgagaacgaccgagacaggaggaaaaccgagagacaaggggaggacgaggcagcagcagcctaggctgctgcgtttttggcgacagaaaaccaccgccagcggcggtggtggtgagcgacggtggtggtggagccgagaaggggttgtggtggtgttagaagtgatggttcatctcaatttcttgataatttgcccttttggaacaataaattgaaggtaactttcaaagctttagatgattatctcttgaaagtagaaagcaagggtaatttagtcttcaattatgttatggagaagggtaataacacccaaatgctaacaaaatcatatagattaatgtgatttgaagttatatagttcatgaatgtgaaggttgactttctgcaatggtcaaacatggacatggacatgattatagcatattagcaatgtaataggaatggaatcaccaattccattgttgaaaatagaaaatttgatgtgtgcagaattttgtaaaatttcccaaaacaccaactttaaggctgcataagtcatgcatgaagaatgcaaatcagtccattctttcgcctaacatcaagtccttgcaacatagattacacaggaaaaagaaacagccaaatccgagttcgtatgaggattgtatgactgttcaacgtttgtcaaaagttgctgtcatgctgtcaaaccagaatttttctaagtctgggaaatatgcagaaaatgggtttgaccagtttttggagcttcataacttgatttatacatatccaaatcagttgattcttgagcctaaactgtagagaaagatgaggagaaggcataggaaaaagaatcaacacatttggatatcgtatacaacctgtgcaaagggttgaagtaggctcaaaacccatttttgacataattgtcaattatatgaattgtgggtaacttttgccgtaatgaaatgttttgacatataagtgtttatattgacttataaacatgttaagactataaacttgataaagtaacatgtcaaaacccgaaaatgcctatttaggcaaagtatgatgcttaattgagaaatcggtcaaatagcataaaaactaaaagtgttctaaatggtccaagaagactttatacataacttacacatcattagcacatgatgtgtagtacatgtgaggtttgaacacttttatacttggaaaattgtcaagtagtcaaaaatgggacaaaagggtaaaattgtccaaaatgagtatatggctaattaaagcaagttgagctactgaaatgaacttagataagtattataaggactataaaagtataatacttggttaactatgagttttgaaggtaatgtcaatttaaagtaagttaaccaaaagaaagtaagttgctaattttgggatatatgacccaaatgagttatgcattggtttttaggttgtgtaatacctataatgagatatgaattgattatgagacatgtattgatgataatacaaatgatgtaatgaaataggttcgttgcctatcgtggagcaaggagcatgcggattagtgtagctagtcttacctagctactaaggtgagtacgtgtggattgttttcccattttcgggtacatggaaaaataatatttttataatgaagtgcctaatgttttgaaagaaagaatgaatgaaaatacttgatgcttttgaaagaatgttttaaaagaaagtaatgttttgaaagaaaaaggtttgatgctttacgtttgaaaatgaatatgtcttgatattaatgaatggaatgggtaaagcatgaatgatataatatgtaataataagatgctaaggatggttatgtttatgatgttcacgtgaatataagaatgttgctcgatctaagatgtcccgggtagggattcagaggagcctatcggggtggtacctccccttcgcgagataggttacctaatgtaaatgatgtcaatgtaatctgatttgttctttttgcattggtacagacttggggtatatcagacccaagtataatatgatatggccatatgataagatgattaaaagagtatgtgaaatgttaaaggttatgtaaaatgttatatgaaattattcaaagagatctttatggttattatgtctatgaaaaggaagcatataatgttatagatattttatgtctaaacccacgtagctcaccagactagttgtctgacgtatttatttttatgccatgtatttcaggttatacacgaggatagactgatgatcgatagaagctagatgttacgacggatagacggagtggagctttcaagttctattgtaatgatcttaccgtaaattcttatttgctttgctatggttgctgtttgatatataattggtaacacccgagaattgttttatatatattcaaaaagtttttatttgggacggtttttgtaaacatgaccaagtgtcatgaaatatttttaaaaccatagcgtttttaaaacgataaaatgaggggtgttacagatgcgataatatatcttaggattatgtcaaatatttatgccaattatacatgagagagcaagtttataagagttttagttattctatcattggatcaatatagatacgtattaatagtagacagagatgcatgaatattatagcagagaactttttgcaaaacctaacctcactgcatgttgcattcatcaaccccacttgtagttgtaacaccatgaatatgccaattaagaaatatcatgaggtatattgtactggaaatgacaaaaagtactaatttacaatagaacgcagtcaaacttggttattttgtaaattttaagaagcaatttacaaaaaataaaacataataaagaaaaacataacagtaaatcacagacattgctcatggaagagtgactgatcaaactgaccgtttattttcttgtttcttgtacaactaccaaacgttcctgtataaccaccaaatagtcccagactttagaaaatagaacagaaaaccacagatatacatacatacagccatacattataatttttaattgaaaaaaatgcatagaaaaccagtctcaaataaagaaccaaataaagaagatacaaatgaaacctggttatcgaataaaagagttagagataatgatttcagcatttgcactcatgtaaacgatatgccattgggatccgttggaaagggtatcgtgaagtgattaaggagaagcggggtgttagggttttttttgcagtttattgaacaggttgatcaaattttgcgccggtagggttaattccttttcgaacgggtttctatctgggtctttcatcaaaatcactcatgtttgtgtctctaacctttagttgcagatggatatcagatcaacggtttcaggctaaatgggggaacaaataacaagaagaaagcggtgacatctgtgttcgtattcgtatggtggaaggggaagagtggcgtatgaattggtcaatgtaaaacatctggaaacaacttctcccaacatgtaaaaggatccggaaaatatgaaaatgacgattagagatttcgttcggatattgggaatatggtcgcgtctgattgccctacggtgcccaccgtttgggtttcttgatttgtcgtttagatagatgtcggaggtctaaattcacaatcctgttcttccgcggtaattgaaattgggtaaggctaattgtttagatcaaatggcagaggacgatgtgaagttgcgttgccctataattggctctcgtgaaattgaattgcaaatttcacaacgatgttagcaacaataatcgtcgctctcgcatcccataccgtcgcaacgtatgatggtgatcgagattgagagatttagggctagatgtggcgttggcggacataggttgtggttgacggaatgaggagaagacgtgagcgtcgattgaggtagcaacaggcggagggttaacggtgtcatcatggtgaaaggtcgcgcgtcgttttatggtcgatagatcatggacgttgtgagtgaatgccggcgcaaacacggtggaggaaagtctatgctttagttgacggggcggttttctctcggagacggaaagaggacgtcgaaagaaaggccggttggagaaaatatattaaacgcgccgacacacgctatcagagaagaccggttttcttatataataatgactaggtacgtgcaagggtcgtgctaagcacgacccatggaggcttcttgagaaaattgtaaacacatatcagataatagcgacgaactttactgttattgatatataaagtgattaagcatggttggccctaagttaaaactcaaactactgtaagtatgtaatggtaataatttatccttttcagctatcgatcttagattgctctgtagtgttgtgtcttagtgcttttcacggttcttttatatgcatatagtcacataaaatcagacacacacatagaataacccacagacaatcacataaaaatcagtctgatgaggtattaatttaagcaataaggattgatattcaagcaattagaggagaataatatattaatttcacgtactcctattgtagttattgtgacaaaacccaactgtttaattgtgccattgcaaaaagtccacctgtttaatttctttatcaacatgtcgttgcatccttttacatgcaaatatttcagaacatcttcctataatgacccttgagatgaactttttagaaatattgttgctggttacattatcagaaaaaacagagcttatgaaaaaagaccaaacatagtaactcgtacttcatgtctggtagttcttttgaatatttccgtcttttgttcgaaacaatttggttgaactgtagtagatctttcaaactgtatacaacttcacaatatgttttgtaagttcattacttctagatcctttcaaaagaagaggtcaaactcaaaatttgcataaaccagatttattgaaggctaattagattagtgttgtagggctttttggatataaataaaaatgaaacataggtgctgcaacaggtgcgaaagaatttggttggacagtagtaggtctttcaaactgtatacaactttacaatatgttttgtaagttcattatttctagatcctttcaaaaaaaaggaggtcaaactaaaaaaatttctaaaccagatttattgaaggctgattagattaacattgcagggctttttgggaataaatataaatgaaagataggtccaacagaaacgaaggaggtgtgaattatgcaacaaatggaaacacacaattatcgtagtacacatgtaattaacagggatcaatttccatttattgttccatcaattgccaaatttaaaagataacaaatgcatcctttttgaatggaaaagtatgatacataaaaccagagagaggtatacatttagtccagaaacggtggaaattactttacctagagttgcgtgaatcaaatggaaaatcagaggaagagaccctaaatcgcaatccatcttctattccctatagaatcaaatggaaatttcttacttccgacctttttcatttccacaagtttgtaatcgataagatcataaaagaaaaccgatttacattagcaaatctttaaggcacttgtttgtagttctcaagtatgcgttacgtatgaaattctagacagaaaagggacgacagggaaaataatggtggaataaataaacaatcaagaatcaaaagataataatccttaaaactaacaaaaattctatcaataagccctacaaatgagaaatatgacggtggagactcacctgattgcagtggcggaagctcttgggggctttggtaggccatggccccctgttttttggaaaattttcaaaaaaaaaaaaattatatatattatatcattgaataagtctagactcaggaaactatgaaagattagaaatagatcaagttatctaaatcaaatctcgaaaaatatggaaggaatcaaaacacaatatttatctcattgtgaaatcaaaaaccctataaaaaacacaaatctaacctacaatttaaatcaaaaacaccataaaaatcaacctaccgtcggattcagccgtcgtcgtcttcttcaccggaccaccatccaccgccattgcttcaatcgtgctttctcgtcaatagcttagtggcttgttcagctttcttgctctaccaatgatttttatacactttccagcaccaccgatttgttgaacggtaatattatgctcctttatatttttgaaatctttgaaagttcatctggttttctgtctctgtaaactgtaaagaaacttttatgttttgttaagaaaatagagagatagagaagaaagagtattgacgtgcttttcccatatgtctcgaatgccacattaggaaaagacaaaaagaaaacaattagtacattacgctagtgtgatagtactgtatgactattagtatgatttacagctttaatagggattcatagaaaagaacaaaataaactacttataaattaaaaatcgagctttatttttaattagtttaaataaatatttagctataacataatatattatatgttttgtatttataatcaaatataattatttgtattacagattagtttgtataatcaaatataatgattcatgtttcgaaaagattgtttaaatatttttatatgccatgttttttaactgaattatatttttttgtatgattttctttacaatttagtttttctttacaatttagttttggccccccttgtttttttgtttgtgttccgcccctgcctgattgatgcgagaaaaggaaagccgccgacatttgtgcttcttcccgcgttaagaaaaatcgcagatgacattacgattctttttctacagaggaataggggggttgcgactttgcgtgatttgttgttaatgagtgatgcgtcgtccgttttgatcggtagcccgtggagactgatttatttgacagatagatgtcgttctcgttgatgatgcaggtgtaattggcggcgttggtgaagcgaagatggcatagggaatggctttgcagaggagaaaaggagtttcacaaatatggattattgaatcgtcgtttcagagtcgcgatggttagtaatcgatggatttgggtttgcacctgttcaaggggtggtgacgtgtcttactgtaagaaagagtcgcgatggttagtaatcgacgatggatttgggtttgcaacatcgttgacggaaacgacgcggcgatggagagaggcggtagagggaatgaaacgggagatttaccttcaaccgcaaacatgtcttcgttcctcgaaaatcatatgcgccctttatgaaaattccttcacgcgccgtacacgcgctcagaaaaaacatagggttttcttatataataatgactaggaacgtttatgcgccgtgataatcacggcccatgagtggtacaaatgttgtaaaacttagaaaatgggaaagttgaatgtacaatgttatgatttattttattcttcatagcacaatacttttgctttagtgaaagcagttaaaaagtggatgtgtcgttagcaactataaacttttaacaaaatattacatacagaacatagaaataagataaacactaaaaaaatacaaaatagccgtgttcaacacggcccacagaatgcaatttggatatttctaaggaggtgtaaaaaatattgctattatgggagctaattctaggtatattttgttactcacatgacatgaatattgagtaattttgtaaaattggtaatttagcttagattttcccacaaagatgagtgttaaaactatcaaggaggatatatggtacttcctcaacaaagatgattttgaaatatctatattttaaatgtaagaactactatcacatgagacaaatattgatattactggttaggattacaacattgatatgtaaaatattttggtaatgtaaattttctaaaaaagcttggaagtagaatgcaacaacaatatatgctagtattacatgttgggtgattttatgaatgtacttacgtgttgatccttatttaccgtacttgaataattttatctgatcaatgctttacctatatggctatatctagcaatgtgcattattcacaaattatggagtgtaaattcatatagacgtatatgacagtggatgaaaaggaaaacaagtaatttgattgaatcactgagaaaaaagcaaaagatacatgcatataaaatgttacatcaaaatacagaagataaatgaaagaaagataataatgtggagcattcaaaataagtaagaatgtaagtccttgatagctcgaaattcttttaagtcttccgtgatagcctaatgtttataatagaagagacattcaaaatgaataagaattatatcacctaacagtatttaatcctactctacttggccattgagcatattgctattgttaaaaattatgtccaaatcgcatttaactgttagatgcaattctcattgccgattacacttatatgtatgtagtaaattataattaactctttgtcctggtcatttgatgtgtcatggatgtttgtaaggtacaaataatagattacaataacttaaatataaaattgagttaatattccagaattcacaaaatgtgtaataacacttatttataatttccacgtaattaatgattaactacttagaatcctacaagtttttgtttatcacctttcatatgatcgtataaatgcaacaacccttaatgcatttatagttgttggtctgccacgaacacctttttctgttgattatttgtcattattatgttaaaaaatgtttgaaaaatgttaaatgggattttgttatggcaaagagatgaacttctccaatattgtagttgcataaatattgccagttccatcatacagaataatacctcttatattgggcaatagaattatagaaacaataagggttaaccgacttacatgtattgatctctgattcttctttacacactggacaaattagtctatcatgtcttgcacaaccctgttaaagaaaacagtgaaaattgaccacgaaggtgtatcctctagtaccaacattacatctatcattttaaaagggaaatgtatacttatcatatatatctaatggacgttgcctttcctccaattgtcactcttacatctaatggaaattatgatcacaatttacttttaacagcatagatacattcatttgttggtaagacacttttggcggattcattgtattaatctttcatgtaaaaattctcattcaagatattaggtttttgttagaacccttgttatttgaacatgcaacctgaattctgattgttcccaacaccaacattaataatacctcttccactagaccatcattccaccttcaggcccttgtgttcattattataagcactattcggtttagttcttttatgagtcctatatttacatccttaaccattttaacaagtccatcatattgggaattttaatataaatacccaatgttattcatttttgtcgcttcagtagatattgaccatactaaagatgagtatttaatactcatacaacatgaaagaaaaaaatgaaaaactaacagtcacttatgtagccccaatctccatacaaatagcgctttaagaataaaccaatttcaggtgaaattgtagaaaggacataaatctgtacatgaaaaaacattaacagttacacataaattatttgcacagagataaaaagcacaatatgtgatgagaagatttaaaagtaggaaagtgaacaaaaataaccatcaaaatccaatgttagtaataaagaaaatgtacgagcgattcataacattcatccaaaacccatatgcgatgttgaatgcatatctggataaaagaatttaataaacaataagcgcctgtaaaatcaataaacaaaatcaataaaaatagacctaaaaatgcaaccgacttattgtttctatacaccaataaatgattttcacgaattttatccaaagaaagataaaaagtcataaaattgaacgggcttaccgatttgaatcgtggtctgtatctgccacgcagatcagataacaatcataatctacatcaaagatttcagaaaaatatgaaacttccgccattactaatttgatatgaatttctcagagcctaaaacaaacctaaattactaaaaatcaaacacaggtttttgacgtttaccttgatttgtgaaaataggcatttgggttttttcgtaggattttctatgaaagaagccatcaagtttatgacattgtaaattaaagcaaagtatgaatatatgaaggataggaaaagagatcggattgtgatgatccggagagaaaaatacacaaaccagagagaagcgatgttgtgattgtggtgtattaatctgtttttaaacctctaaccacttcttgtaagtccgtaatcagacgaaatgcgcttgaataataagtctttcacgccagtgtcgaggattaatgccgtcaagggtggtggggaggtatttataggaagaaatgcggtggaggtgaatggaggcgtcgatcaaacccaagaggaaacggtttgtcgaaatagatgcgttggtcattcgccataagtttgtgaaacggtgagtgtatccttggcgtcgtttgaaagtttgtgaaacggtggcgatgctccgttaaaggaccggcaacggatcgagcgatatgtaaatgggagggcagaaatattgcatacgcgccggacacgcgcttatggaagtgatagtatggaagtgataggattttcttatataataatgatataacttaattttaaataacatcgtactatacgtttcaaaataaattagagttttaataaaattacggaatgattaaaccaattacaaacaaatataagtgtcggtgatataaaatttgtaacaacgaaaacatttcaaaaaatctgaatgcggaaatccataacatgagaatacgattcgtttgcagtcacgccggtttcttcaccttctgagggtcctgaaaaaggttaataaacaatgggtaagctaaagtttagtgatcatacacattacttgtctattatatgagttagatacattcacatatcacatacatacaagcatacacatattacaaaaaatcctcacacatattgttggtaccaactactatttagtggaccaggttattcttgggggtcaattgtggttaaccataaatgtttggagtttaggttccccttgggggccgaccataaacacccatatcatatctctgtctaggttctctttggggactgactatagacaactttgctcagattctccttggaggataaccatcctcgacgagattaacaacggaccaccactgttttcagcttgtaacctgcaacatcaatagttgcgatcttcggcgacaatgatgaaccaacatcaacacaacagtagtgccggtatgttagtcgttggataatagaggcatcatctgacccttttagcggtgacttttgttgcctatgaaggtcttatcagttatatatcgattaggcttggttctaggttataagaaaaaatgattctctatccggtatgacatagagacaaaccggtctacttttataagaaaatcatatttgaaacaatgtcgtgttaataattaatatgtgtatgtttaaatttagaatatcctgaacttatatttagttcgcaaccctatgtaagatcgaactaattttaaaccgtaaccagacgtgcatgtttgatcagtaataaaaaggctatcatcgtcgaaaatattaaaacaaaatataatatcttatttggcgccatttc

SEQ ID NO: 8 - DNA, 684 bp, Taraxacum officinale
  source 1..684: mol_type = unassigned DNA
atgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatggcagataatggcaacaccggccgtcaaaaggatgacgacggtggccatgatgggggacgcccaaatccgaccaccccaccctccccttcccgcaccccgcgaagacccaggcggaccacatcgccgcctaaacattctccgggggcgtcttcaagcaccatgccagcgccgcctactccccctgcgccgacgggaatcaccggtgcgtctagttcttctgtgggtactaatataatttcatttactccacccaaaaccaaaagaacgaagtcggtgatttgtccgatctgtaagaaagatatgtgccatgagaaggcgctgtgtggccacatccggtggcatactcaggaagaaagattggcggccagcattgctatagcaagagcgctatcttctaacgttgttgtttctggcaatggcgatgaagatgaaggtccatctaaaaagtataaactcccggacctgaacaagtcaccaccgccggaggaggaggacgaggacgctgcctga

SEQ ID NO: 9 - DNA, 4521 bp, Taraxacum officinale
  source 1..4521: mol_type = unassigned DNA
ttttcttgcgggagaggcatgtatgtgtggcctttttgcaataatactatgcatgggagatgcataaagtttatctttattttgttttagtttaataatgtgtggcaaactttatatcatttatgatttcggccatgtactgctacatcgttctaggatggttattccctagaatttgcttttatatattttttttcttcgttaattcttctcttcctttaacgttatttcatgtgaatttgcgatatcatgatattgcaggaacgtaacttagaactcaacgataaaaagtgtgttgagatcgtggaagttgatcgtttatcttgaattcacaagataagcaacatgagatagaataattaattactgcggagatttctttttagattcatcttattcgttgtaaattgcaatgcaaatttttaagtagaataacgaaagcgtgttgttatttacggttaaatgcaaaaaatcattaacatatctccataattgatttgtttagtatgcattctcatgtgttctattgcaaaattctattttgaaattcaattaaagcaatgcattttagatataaagcaattccatcacttaattatattgggtagatttttaccaaatcactgtcctaaaagaaaaataaaaaatgaaagttcaatgccatagtgatcccgtcaattatgtgatcaatttttaccagagcaaaaccccaaaagttaaagagtataattgcagaacgacaacaatctgaggatgatactaactccggtataaaccgaatgcttttgtggggatttcactaatcaactatttggctctcatcacaaggccgtgttaatggattactattagttataaggccatcccaagttgtttggctatattattaccttttaaaaaccaaaatgtgtaaactgtaaaattacacttttggttcttgtattttctaatttttagaattatgtatttggttcttgtagtttttttatttgtatgaatcatattagttttggtccacgataacttttattttctataattacacttttggtcattgtagttttaatttgcaagaaaaaaataatctatctttttagtccatatatatagtttgcaatattctatattttaggttcaggttatatatcttttacaaaaatactacttatgattttttaatgaatatatcatatttcgtaaattacataaacattttatataattttatttagtcagaactataggattcgcatgataactttgctgaacttaattgggcttcatttttaacaaaggataatgcatcatgcatgacataaaaatttgtaatgtttctaatgtgcatgcactacaagaacctgacccttcatatacattaatgaatgattctcttttcttattatatcttagcaaacggtttggataagagtctgaacaatattaagaattgcactagctagctagtccatgcagcaagcaaattaattagaggactttttaagaggcttgatcacttcattgtatatctaatggcactgtggatatatgattggattgtgtgtatgtatggaatttaatattactaaaaaatattaatgacaaacaataacacactaatttataacttacaaaagcgtgtgtcattaaagaaaatgtcatgtttcttaaaatatggagaatttactacttacataattatgtgtcataaatttatgtcatcttgtgtgatatttatgacttacaaaaatgtatgttatgtttatcgtcataaaatctgtaaaaaaaaatagaaacatggaggatactttcaaggtgtagaaacttgacagcataaacgggtttattaccttcatcaaaacttatcttacttcaatctcttggagatgcaggttgattttaatgctaaatgaacttttcaaatgtataaatatatatcttttgacggatttgacattgatggagatgaagatacaccagtgaattcacctttcaatgatcatactgacataccaagtactcttatcccctctccacctccacagaacgaacctcaatttcaaaaacgtaactgccgctgttctaaaaacagaaaattttcaaaaacgtaactatcatttaaatatttaatcatttttcaattggattcatgctgtaatagaagttactggactttaaacttcttgtagctgatgagattcatgaacaacagtccaaggagattttccaaatcaacaagtttctgatgagaacaaggtaaaaaaaataagtttttcatttaaacatatgattttggttttcaaaagataattaattataggaactcttctgaaaagtcccaaagtttacccgtaaaattgattttggtttttttttaaattgaataaaaaagtccataaaattggataaattattcgatttagcccaatttacctagtatatctggtaaaaattggttaaatcgaataatttaggcagtttgttgggcttttttgcgcaaaaacattattttgggacttaatcgttaattttccccaagtttcagtttatttacagtattggaataaaaccgaacctagtgcgctaattttgtcttttggtgtttagaattatttgggtttattggtctcggtttctaaacgagttcggtttttggtttcttgattcgatttttggcttttgggttcaacccatggattttcgatttggttttggttttgataatattttttgtgcaatcgttcaaaaaatccgagttagattgggtttaaatgttatcaaaaccaaaaccgatggtagaaccccaaaactgaacccaatcgaaaatccatgggttgaacctcaaaaacgaacccaataaattctaaaaaccaaaatccaaaattagcggataaaaattttaaaatacatatctactggtaaggtgtttttgacaatttatgtaaattatagaaaaaaatgtattaagtgtttgctatttaagataaaaatggcgttaaaaatagaattggttagaaatgaaccatttttttatattttttacttcatttattaagtaaatagtagaggtgaattagagatgatctaagatattttttttctttcaaaaagagcttcgaaaacacttttccgacagaaacaagacacaaacctttttactcgtagaccgacgatttttttaaagatcaaagacctaagaagatttgtctcccatcttcttaaaacatatgtgagatagacacttctagcctcttctatgctaaaaaaaagagctcgcctttagtttggttaatttgagacaatggtctccacaaaaacataggtagttaggtactcacgcgccaagttactaatctcaaatcaaacataactgtatgatcgtcaacacattttacaagttaacaaaagagcattgctttagacttaaaaattgctttgagaaaaaacaaacacctaagaatttctaaaagcgatgatttcaactctctttaaaatgtatatatatgttacattttggtctttaaacttttcaagaaagtacaattcaactcctattttcaaaatgttcttacatgagcgatatgattttaccgtttaaaatgtaaaccatgataactatttctaaattccaatttttgtttcgttgatcaatctcattatcattcaatgcatacaaaacacaatacaatctgttttgtcatcttcttcaaggccgaaagacaactaaacatatgaagactgaagaggacaatctagcaaactcaaacttcactataaatttgcaattgctattggtagccatgaagtaacatttttgaggaggttaacaaaattttaacagtaaattcaataatccatcaccacgatatgtaatcatctacacaaacacagatgatgatagtcatatatgttcgtaatcacacaagcaagaaatcgcaatgtactttacaccgtcttctctgacttgaacacgggaattggttgtctaagaagtagattgtttcccacggatttaccaagtacatttaaatccttcaccctttgtatctctcttcctgttttttgttttgcaatcaacaaaaaaaaaagtaaaaatcccatcttaaattcacgcaaaaaaacacattgaatcaaccatcactatagtagtttcaagaaaaatctgcacttccataatttctttctattacaccattatactaggaaaacttcatagcaatattatctaaatacaaagcaatttttactagtaatattcgtagatttgtcaatatatagtgtcctaataaagaaaaaataacggaactataatgctctaataggtagattttttttagtataatgtcctaataagagaaaaaacaaatgtacaattatgtaataataagaaaagaaagtaatatgcttaatagacaaataaatgaaaccatgttgctatttcttgcatctagtccttatttgaataatatgtagcatattaactacttatgaattttatatataaatgataaaaaattccgtgag

SEQ ID NO: 10 - DNA, 19724 bp, Taraxacum officinale
  source 1..19724: mol_type = unassigned DNA
ctgacttggccaacattttttgttcttccgatcccttcgtaacaatcagtttaagcacaattgaattatatggagataaataggtgaaatttggaagacgaggatgggagagtgcgaagagccaaagaagagaaatgtgggatagcggccggccggttgttaatagaagtgcttagatcttggagaccttctaatgcttatggaggaaccagatgcttcaatttgtaaatctgcgctgcccacgtagaatattgaaaaagaaaatcaaatcgatcaataatgcgaccatatactttaggatttcccacttgtataattctcttattcattttcaaatctttatctaaattaaggatatcggcaaaaatataatcctaattgtttttcaaaatgataaggattataaggtaacttaattcttgttttattcatatttaattattattgttttttatcaatgttttaaaacccgggttttgagtcaacccggtcttgtgaaaaatcccgggtcagccggtcagctgatctaccggttcaatatatttaaataaaatataaattttcatatagaatttgagttttaggctaagaaaatcgggttttgggctaaggaaaatcgggtttaagttttgggctaatggattttcgggtcaataacgtttttttccgttttttagatcggttcgaccgcgttttgtgtgaaacccggccgggttgatccgagtcaataattggcaacaacccggtatatgttgacccgctcttgtccccgggtcccggttcaaccggttggatcggccgggttgaccctggttttaaaacactgttttttatacaatatatttgtttttgtttttagttttgtttttgtttttgattttgtagatatatttttaattttatttttattttgtataaatatagtcattacaaccgattatttgtaagttgagtcggtcaaaacgaactttctgataattcttatacatttttcggcgaaacatgaatattttgatcaaccaatatgcttttttctaggttttttaaataaaccgcacgttcaattgcatattcgcaaacaatagaactcgaaaaccctaatcgagcttgttttatagaaatctgggttgagtgcatacataataattaataactgtgaaaaagtttgttttaacatgctaaacctatcgatagtcggttataataattatatgtaaacaaaatcagaaacaataatgatatatgtcacccaaaaaaatagttactaaatatatacaaaatcaaaaacaacagtaatttcttttacaaaaaaaagagaacaatagtgactaactttagaaagaaaaaaaaagaactaaattactcttataaatcattatcttttatataaattggtaaattggtcatgtaatctagaatgtgaattgcggaatgctttagtcacattttgaacaaactggtaattattaatttcgataaaagttcttcaagaatagtttggtcacattttggacgacaatggtgtgttttccatagtaagcaatacgtaatgagttcgcttaataggaaaatagatctaaagcttaatgaaatcatatgtataagtaacatatatttttcacacgttacattgtttgtaacgtcctcaatttttttaatatatataactaggtacactctaaggtcgtgctaagcacgacccacgaatggtaatttaggtaatttttcatccttgactaaaatacattatgctaataacaaagaattaagaatacaatgctataatgcataagtaaataaaagtacatgatatttctcacatttagtctttatacatagaaaacttacactggccatttgcaaaaaaacattaaatctccctaaaaagtagagtttacaaaatgaaagcttgtttttatagcaaacaataatcaatatgtgaagctatagtatagtaacctactcatttcaacttttaggcagtgcagtaattacttactcatgttaatggtgaaatgcattaagataaacttcattcaacggattaaatctgggttcattataaatgtactcttagttactcctccatgtaactgcatttagcgaacaacaaaagctctaataaataatatgaaacacatgctaatttataaatgacatcatgggtatagatgctgtaacatcctaaacttttaaaataaataataaatcatatttgggatttaaagtaatattttaagataattgataaataagagaatgaaatacaaagaggcattttatttttagcctttgatttataaaaaagaaaaaaccttgaaattggatgtattttgaaatgggtaagtattttaaaagatgtgtaagttaagggggataaatggaaattaaggagataagcatttgaccaaagtcaaaatagaaggaacaataagtcggtcgattgttttcctgtgtgcatcttcggacgacagagagacagaaaagaacgagcgagagacagagaggaatcgagacagagagattatcgagagagagacagcgaagtcgggagagggagaccgagaacgaccgagacaggaggaaaaccgagagacaaggggaggacgaggcagcagcagcctaggctgctgcgtttttggcgacagaaaaccaccgccagcggcggtggtggtgagcgacggtggtggtggagccgagaaggggttgtggtggtgttagaagtgatggttcatctcaatttcttgataatttgcccttttggaacaataaattgaaggtaactttcaaagctttagatgattatctcttgaaagtagaaagcaagggtaatttagtcttcaattatgttatggagaagggtaataacacccaaatgctaacaaaatcatatagattaatgtgatttgaagttatatagttcatgaatgtgaaggttgactttctgcaatggtcaaacatggacatggacatgattatagcatattagcaatgtaataggaatggaatcaccaattccattgttgaaaatagaaaatttgatgtgtgcagaattttgtaaaatttcccaaaacaccaactttaaggctgcataagtcatgcatgaagaatgcaaatcagtccattctttcgcctaacatcaagtccttgcaacatagattacacaggaaaaagaaacagccaaatccgagttcgtatgaggattgtatgactgttcaacgtttgtcaaaagttgctgtcatgctgtcaaaccagaatttttctaagtctgggaaatatgcagaaaatgggtttgaccagtttttggagcttcataacttgatttatacatatccaaatcagttgattcttgagcctaaactgtagagaaagatgaggagaaggcataggaaaaagaatcaacacatttggatatcgtatacaacctgtgcaaagggttgaagtaggctcaaaacccatttttgacataattgtcaattatatgaattgtgggtaacttttgccgtaatgaaatgttttgacatataagtgtttatattgacttataaacatgttaagactataaacttgataaagtaacatgtcaaaacccgaaaatgcctatttaggcaaagtatgatgcttaattgagaaatcggtcaaatagcataaaaactaaaagtgttctaaatggtccaagaagactttatacataacttacacatcattagcacatgatgtgtagtacatgtgaggtttgaacacttttatacttggaaaattgtcaagtagtcaaaaatgggacaaaagggtaaaattgtccaaaatgagtatatggctaattaaagcaagttgagctactgaaatgaacttagataagtattataaggactataaaagtataatacttggttaactatgagttttgaaggtaatgtcaatttaaagtaagttaaccaaaagaaagtaagttgctaattttgggatatatgacccaaatgagttatgcattggtttttaggttgtgtaatacctataatgagatatgaattgattatgagacatgtattgatgataatacaaatgatgtaatgaaataggttcgttgcctatcgtggagcaaggagcatgcggattagtgtagctagtcttacctagctactaaggtgagtacgtgtggattgttttcccattttcgggtacatggaaaaataatatttttataatgaagtgcctaatgttttgaaagaaagaatgaatgaaaatacttgatgcttttgaaagaatgttttaaaagaaagtaatgttttgaaagaaaaaggtttgatgctttacgtttgaaaatgaatatgtcttgatattaatgaatggaatgggtaaagcatgaatgatataatatgtaataataagatgctaaggatggttatgtttatgatgttcacgtgaatataagaatgttgctcgatctaagatgtcccgggtagggattcagaggagcctatcggggtggtacctccccttcgcgagataggttacctaatgtaaatgatgtcaatgtaatctgatttgttctttttgcattggtacagacttggggtatatcagacccaagtataatatgatatggccatatgataagatgattaaaagagtatgtgaaatgttaaaggttatgtaaaatgttatatgaaattattcaaagagatctttatggttattatgtctatgaaaaggaagcatataatgttatagatattttatgtctaaacccacgtagctcaccagactagttgtctgacgtatttatttttatgccatgtatttcaggttatacacgaggatagactgatgatcgatagaagctagatgttacgacggatagacggagtggagctttcaagttctattgtaatgatcttaccgtaaattcttatttgctttgctatggttgctgtttgatatataattggtaacacccgagaattgttttatatatattcaaaaagtttttatttgggacggtttttgtaaacatgaccaagtgtcatgaaatatttttaaaaccatagcgtttttaaaacgataaaatgaggggtgttacagatgcgataatatatcttaggattatgtcaaatatttatgccaattatacatgagagagcaagtttataagagttttagttattctatcattggatcaatatagatacgtattaatagtagacagagatgcatgaatattatagcagagaactttttgcaaaacctaacctcactgcatgttgcattcatcaaccccacttgtagttgtaacaccatgaatatgccaattaagaaatatcatgaggtatattgtactggaaatgacaaaaagtactaatttacaatagaacgcagtcaaacttggttattttgtaaattttaagaagcaatttacaaaaaataaaacataataaagaaaaacataacagtaaatcacagacattgctcatggaagagtgactgatcaaactgaccgtttattttcttgtttcttgtacaactaccaaacgttcctgtataaccaccaaatagtcccagactttagaaaatagaacagaaaaccacagatatacatacatacagccatacattataatttttaattgaaaaaaatgcatagaaaaccagtctcaaataaagaaccaaataaagaagatacaaatgaaacctggttatcgaataaaagagttagagataatgatttcagcatttgcactcatgtaaacgatatgccattgggatccgttggaaagggtatcgtgaagtgattaaggagaagcggggtgttagggttttttttgcagtttattgaacaggttgatcaaattttgcgccggtagggttaattccttttcgaacgggtttctatctgggtctttcatcaaaatcactcatgtttgtgtctctaacctttagttgcagatggatatcagatcaacggtttcaggctaaatgggggaacaaataacaagaagaaagcggtgacatctgtgttcgtattcgtatggtggaaggggaagagtggcgtatgaattggtcaatgtaaaacatctggaaacaacttctcccaacatgtaaaaggatccggaaaatatgaaaatgacgattagagatttcgttcggatattgggaatatggtcgcgtctgattgccctacggtgcccaccgtttgggtttcttgatttgtcgtttagatagatgtcggaggtctaaattcacaatcctgttcttccgcggtaattgaaattgggtaaggctaattgtttagatcaaatggcagaggacgatgtgaagttgcgttgccctataattggctctcgtgaaattgaattgcaaatttcacaacgatgttagcaacaataatcgtcgctctcgcatcccataccgtcgcaacgtatgatggtgatcgagattgagagatttagggctagatgtggcgttggcggacataggttgtggttgacggaatgaggagaagacgtgagcgtcgattgaggtagcaacaggcggagggttaacggtgtcatcatggtgaaaggtcgcgcgtcgttttatggtcgatagatcatggacgttgtgagtgaatgccggcgcaaacacggtggaggaaagtctatgctttagttgacggggcggttttctctcggagacggaaagaggacgtcgaaagaaaggccggttggagaaaatatattaaacgcgccgacacacgctatcagagaagaccggttttcttatataataatgactaggtacgtgcaagggtcgtgctaagcacgacccatggaggcttcttgagaaaattgtaaacacatatcagataatagcgacgaactttactgttattgatatataaagtgattaagcatggttggccctaagttaaaactcaaactactgtaagtatgtaatggtaataatttatccttttcagctatcgatcttagattgctctgtagtgttgtgtcttagtgcttttcacggttcttttatatgcatatagtcacataaaatcagacacacacatagaataacccacagacaatcacataaaaatcagtctgatgaggtattaatttaagcaataaggattgatattcaagcaattagaggagaataatatattaatttcacgtactcctattgtagttattgtgacaaaacccaactgtttaattgtgccattgcaaaaagtccacctgtttaatttctttatcaacatgtcgttgcatccttttacatgcaaatatttcagaacatcttcctataatgacccttgagatgaactttttagaaatattgttgctggttacattatcagaaaaaacagagcttatgaaaaaagaccaaacatagtaactcgtacttcatgtctggtagttcttttgaatatttccgtcttttgttcgaaacaatttggttgaactgtagtagatctttcaaactgtatacaacttcacaatatgttttgtaagttcattacttctagatcctttcaaaagaagaggtcaaactcaaaatttgcataaaccagatttattgaaggctaattagattagtgttgtagggctttttggatataaataaaaatgaaacataggtgctgcaacaggtgcgaaagaatttggttggacagtagtaggtctttcaaactgtatacaactttacaatatgttttgtaagttcattatttctagatcctttcaaaaaaaaggaggtcaaactaaaaaaatttctaaaccagatttattgaaggctgattagattaacattgcagggctttttgggaataaatataaatgaaagataggtccaacagaaacgaaggaggtgtgaattatgcaacaaatggaaacacacaattatcgtagtacacatgtaattaacagggatcaatttccatttattgttccatcaattgccaaatttaaaagataacaaatgcatcctttttgaatggaaaagtatgatacataaaaccagagagaggtatacatttagtccagaaacggtggaaattactttacctagagttgcgtgaatcaaatggaaaatcagaggaagagaccctaaatcgcaatccatcttctattccctatagaatcaaatggaaatttcttacttccgacctttttcatttccacaagtttgtaatcgataagatcataaaagaaaaccgatttacattagcaaatctttaaggcacttgtttgtagttctcaagtatgcgttacgtatgaaattctagacagaaaagggacgacagggaaaataatggtggaataaataaacaatcaagaatcaaaagataataatccttaaaactaacaaaaattctatcaataagccctacaaatgagaaatatgacggtggagactcacctgattgcagtggcggaagctcttgggggctttggtaggccatggccccctgttttttggaaaattttcaaaaaaaaaaaaattatatatattatatcattgaataagtctagactcaggaaactatgaaagattagaaatagatcaagttatctaaatcaaatctcgaaaaatatggaaggaatcaaaacacaatatttatctcattgtgaaatcaaaaaccctataaaaaacacaaatctaacctacaatttaaatcaaaaacaccataaaaatcaacctaccgtcggattcagccgtcgtcgtcttcttcaccggaccaccatccaccgccattgcttcaatcgtgctttctcgtcaatagcttagtggcttgttcagctttcttgctctaccaatgatttttatacactttccagcaccaccgatttgttgaacggtaatattatgctcctttatatttttgaaatctttgaaagttcatctggttttctgtctctgtaaactgtaaagaaacttttatgttttgttaagaaaatagagagatagagaagaaagagtattgacgtgcttttcccatatgtctcgaatgccacattaggaaaagacaaaaagaaaacaattagtacattacgctagtgtgatagtactgtatgactattagtatgatttacagctttaatagggattcatagaaaagaacaaaataaactacttataaattaaaaatcgagctttatttttaattagtttaaataaatatttagctataacataatatattatatgttttgtatttataatcaaatataattatttgtattacagattagtttgtataatcaaatataatgattcatgtttcgaaaagattgtttaaatatttttatatgccatgttttttaactgaattatatttttttgtatgattttctttacaatttagtttttctttacaatttagttttggccccccttgtttttttgtttgtgttccgcccctgcctgattgatgcgagaaaaggaaagccgccgacatttgtgcttcttcccgcgttaagaaaaatcgcagatgacattacgattctttttctacagaggaataggggggttgcgactttgcgtgatttgttgttaatgagtgatgcgtcgtccgttttgatcggtagcccgtggagactgatttatttgacagatagatgtcgttctcgttgatgatgcaggtgtaattggcggcgttggtgaagcgaagatggcatagggaatggctttgcagaggagaaaaggagtttcacaaatatggattattgaatcgtcgtttcagagtcgcgatggttagtaatcgatggatttgggtttgcacctgttcaaggggtggtgacgtgtcttactgtaagaaagagtcgcgatggttagtaatcgacgatggatttgggtttgcaacatcgttgacggaaacgacgcggcgatggagagaggcggtagagggaatgaaacgggagatttaccttcaaccgcaaacatgtcttcgttcctcgaaaatcatatgcgccctttatgaaaattccttcacgcgccgtacacgcgctcagaaaaaacatagggttttcttatataataatgactaggaacgtttatgcgccgtgataatcacggcccatgagtggtacaaatgttgtaaaacttagaaaatgggaaagttgaatgtacaatgttatgatttattttattcttcatagcacaatacttttgctttagtgaaagcagttaaaaagtggatgtgtcgttagcaactataaacttttaacaaaatattacatacagaacatagaaataagataaacactaaaaaaatacaaaatagccgtgttcaacacggcccacagaatgcaatttggatatttctaaggaggtgtaaaaaatattgctattatgggagctaattctaggtatattttgttactcacatgacatgaatattgagtaattttgtaaaattggtaatttagcttagattttcccacaaagatgagtgttaaaactatcaaggaggatatatggtacttcctcaacaaagatgattttgaaatatctatattttaaatgtaagaactactatcacatgagacaaatattgatattactggttaggattacaacattgatatgtaaaatattttggtaatgtaaattttctaaaaaagcttggaagtagaatgcaacaacaatatatgctagtattacatgttgggtgattttatgaatgtacttacgtgttgatccttatttaccgtacttgaataattttatctgatcaatgctttacctatatggctatatctagcaatgtgcattattcacaaattatggagtgtaaattcatatagacgtatatgacagtggatgaaaaggaaaacaagtaatttgattgaatcactgagaaaaaagcaaaagatacatgcatataaaatgttacatcaaaatacagaagataaatgaaagaaagataataatgtggagcattcaaaataagtaagaatgtaagtccttgatagctcgaaattcttttaagtcttccgtgatagcctaatgtttataatagaagagacattcaaaatgaataagaattatatcacctaacagtatttaatcctactctacttggccattgagcatattgctattgttaaaaattatgtccaaatcgcatttaactgttagatgcaattctcattgccgattacacttatatgtatgtagtaaattataattaactctttgtcctggtcatttgatgtgtcatggatgtttgtaaggtacaaataatagattacaataacttaaatataaaattgagttaatattccagaattcacaaaatgtgtaataacacttatttataatttccacgtaattaatgattaactacttagaatcctacaagtttttgtttatcacctttcatatgatcgtataaatgcaacaacccttaatgcatttatagttgttggtctgccacgaacacctttttctgttgattatttgtcattattatgttaaaaaatgtttgaaaaatgttaaatgggattttgttatggcaaagagatgaacttctccaatattgtagttgcataaatattgccagttccatcatacagaataatacctcttatattgggcaatagaattatagaaacaataagggttaaccgacttacatgtattgatctctgattcttctttacacactggacaaattagtctatcatgtcttgcacaaccctgttaaagaaaacagtgaaaattgaccacgaaggtgtatcctctagtaccaacattacatctatcattttaaaagggaaatgtatacttatcatatatatctaatggacgttgcctttcctccaattgtcactcttacatctaatggaaattatgatcacaatttacttttaacagcatagatacattcatttgttggtaagacacttttggcggattcattgtattaatctttcatgtaaaaattctcattcaagatattaggtttttgttagaacccttgttatttgaacatgcaacctgaattctgattgttcccaacaccaacattaataatacctcttccactagaccatcattccaccttcaggcccttgtgttcattattataagcactattcggtttagttcttttatgagtcctatatttacatccttaaccattttaacaagtccatcatattgggaattttaatataaatacccaatgttattcatttttgtcgcttcagtagatattgaccatactaaagatgagtatttaatactcatacaacatgaaagaaaaaaatgaaaaactaacagtcacttatgtagccccaatctccatacaaatagcgctttaagaataaaccaatttcaggtgaaattgtagaaaggacataaatctgtacatgaaaaaacattaacagttacacataaattatttgcacagagataaaaagcacaatatgtgatgagaagatttaaaagtaggaaagtgaacaaaaataaccatcaaaatccaatgttagtaataaagaaaatgtacgagcgattcataacattcatccaaaacccatatgcgatgttgaatgcatatctggataaaagaatttaataaacaataagcgcctgtaaaatcaataaacaaaatcaataaaaatagacctaaaaatgcaaccgacttattgtttctatacaccaataaatgattttcacgaattttatccaaagaaagataaaaagtcataaaattgaacgggcttaccgatttgaatcgtggtctgtatctgccacgcagatcagataacaatcataatctacatcaaagatttcagaaaaatatgaaacttccgccattactaatttgatatgaatttctcagagcctaaaacaaacctaaattactaaaaatcaaacacaggtttttgacgtttaccttgatttgtgaaaataggcatttgggttttttcgtaggattttctatgaaagaagccatcaagtttatgacattgtaaattaaagcaaagtatgaatatatgaaggataggaaaagagatcggattgtgatgatccggagagaaaaatacacaaaccagagagaagcgatgttgtgattgtggtgtattaatctgtttttaaacctctaaccacttcttgtaagtccgtaatcagacgaaatgcgcttgaataataagtctttcacgccagtgtcgaggattaatgccgtcaagggtggtggggaggtatttataggaagaaatgcggtggaggtgaatggaggcgtcgatcaaacccaagaggaaacggtttgtcgaaatagatgcgttggtcattcgccataagtttgtgaaacggtgagtgtatccttggcgtcgtttgaaagtttgtgaaacggtggcgatgctccgttaaaggaccggcaacggatcgagcgatatgtaaatgggagggcagaaatattgcatacgcgccggacacgcgcttatggaagtgatagtatggaagtgataggattttcttatataataatgatataacttaattttaaataacatcgtactatacgtttcaaaataaattagagttttaataaaattacggaatgattaaaccaattacaaacaaatataagtgtcggtgatataaaatttgtaacaacgaaaacatttcaaaaaatctgaatgcggaaatccataacatgagaatacgattcgtttgcagtcacgccggtttcttcaccttctgagggtcctgaaaaaggttaataaacaatgggtaagctaaagtttagtgatcatacacattacttgtctattatatgagttagatacattcacatatcacatacatacaagcatacacatattacaaaaaatcctcacacatattgttggtaccaactactatttagtggaccaggttattcttgggggtcaattgtggttaaccataaatgtttggagtttaggttccccttgggggccgaccataaacacccatatcatatctctgtctaggttctctttggggactgactatagacaactttgctcagattctccttggaggataaccatcctcgacgagattaacaacggaccaccactgttttcagcttgtaacctgcaacatcaatagttgcgatcttcggcgacaatgatgaaccaacatcaacacaacagtagtgccggtatgttagtcgttggataatagaggcatcatctgacccttttagcggtgacttttgttgcctatgaaggtcttatcagttatatatcgattaggcttggttctaggttataagaaaaaatgattctctatccggtatgacatagagacaaaccggtctacttttataagaaaatcatatttgaaacaatgtcgtgttaataattaatatgtgtatgtttaaatttagaatatcctgaacttatatttagttcgcaaccctatgtaagatcgaactaattttaaaccgtaaccagacgtgcatgtttgatcagtaataaaaaggctatcatcgtcgaaaatattaaaacaaaatataatatcttatttggcgccatttcatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatggcagataatggcaacaccggccgtcaaaaggatgacgacggtggccatgatgggggacgcccaaatccgaccaccccaccctccccttcccgcaccccgcgaagacccaggcggaccacatcgccgcctaaacattctccgggggcgtcttcaagcaccatgccagcgccgcctactccccctgcgccgacgggaatcaccggtgcgtctagttcttctgtgggtactaatataatttcatttactccacccaaaaccaaaagaacgaagtcggtgatttgtccgatctgtaagaaagatatgtgccatgagaaggcgctgtgtggccacatccggtggcatactcaggaagaaagattggcggccagcattgctatagcaagagcgctatcttctaacgttgttgtttctggcaatggcgatgaagatgaaggtccatctaaaaagtataaactcccggacctgaacaagtcaccaccgccggaggaggaggacgaggacgctgcctgattttcttgcgggagaggcatgtatgtgtggcctttttgcaataatactatgcatgggagatgcataaagtttatctttattttgttttagtttaataatgtgtggcaaactttatatcatttatgatttcggccatgtactgctacatcgttctaggatggttattccctagaatttgcttttatatattttttttcttcgttaattcttctcttcctttaacgttatttcatgtgaatttgcgatatcatgatattgcaggaacgtaacttagaactcaacgataaaaagtgtgttgagatcgtggaagttgatcgtttatcttgaattcacaagataagcaacatgagatagaataattaattactgcggagatttctttttagattcatcttattcgttgtaaattgcaatgcaaatttttaagtagaataacgaaagcgtgttgttatttacggttaaatgcaaaaaatcattaacatatctccataattgatttgtttagtatgcattctcatgtgttctattgcaaaattctattttgaaattcaattaaagcaatgcattttagatataaagcaattccatcacttaattatattgggtagatttttaccaaatcactgtcctaaaagaaaaataaaaaatgaaagttcaatgccatagtgatcccgtcaattatgtgatcaatttttaccagagcaaaaccccaaaagttaaagagtataattgcagaacgacaacaatctgaggatgatactaactccggtataaaccgaatgcttttgtggggatttcactaatcaactatttggctctcatcacaaggccgtgttaatggattactattagttataaggccatcccaagttgtttggctatattattaccttttaaaaaccaaaatgtgtaaactgtaaaattacacttttggttcttgtattttctaatttttagaattatgtatttggttcttgtagtttttttatttgtatgaatcatattagttttggtccacgataacttttattttctataattacacttttggtcattgtagttttaatttgcaagaaaaaaataatctatctttttagtccatatatatagtttgcaatattctatattttaggttcaggttatatatcttttacaaaaatactacttatgattttttaatgaatatatcatatttcgtaaattacataaacattttatataattttatttagtcagaactataggattcgcatgataactttgctgaacttaattgggcttcatttttaacaaaggataatgcatcatgcatgacataaaaatttgtaatgtttctaatgtgcatgcactacaagaacctgacccttcatatacattaatgaatgattctcttttcttattatatcttagcaaacggtttggataagagtctgaacaatattaagaattgcactagctagctagtccatgcagcaagcaaattaattagaggactttttaagaggcttgatcacttcattgtatatctaatggcactgtggatatatgattggattgtgtgtatgtatggaatttaatattactaaaaaatattaatgacaaacaataacacactaatttataacttacaaaagcgtgtgtcattaaagaaaatgtcatgtttcttaaaatatggagaatttactacttacataattatgtgtcataaatttatgtcatcttgtgtgatatttatgacttacaaaaatgtatgttatgtttatcgtcataaaatctgtaaaaaaaaatagaaacatggaggatactttcaaggtgtagaaacttgacagcataaacgggtttattaccttcatcaaaacttatcttacttcaatctcttggagatgcaggttgattttaatgctaaatgaacttttcaaatgtataaatatatatcttttgacggatttgacattgatggagatgaagatacaccagtgaattcacctttcaatgatcatactgacataccaagtactcttatcccctctccacctccacagaacgaacctcaatttcaaaaacgtaactgccgctgttctaaaaacagaaaattttcaaaaacgtaactatcatttaaatatttaatcatttttcaattggattcatgctgtaatagaagttactggactttaaacttcttgtagctgatgagattcatgaacaacagtccaaggagattttccaaatcaacaagtttctgatgagaacaaggtaaaaaaaataagtttttcatttaaacatatgattttggttttcaaaagataattaattataggaactcttctgaaaagtcccaaagtttacccgtaaaattgattttggtttttttttaaattgaataaaaaagtccataaaattggataaattattcgatttagcccaatttacctagtatatctggtaaaaattggttaaatcgaataatttaggcagtttgttgggcttttttgcgcaaaaacattattttgggacttaatcgttaattttccccaagtttcagtttatttacagtattggaataaaaccgaacctagtgcgctaattttgtcttttggtgtttagaattatttgggtttattggtctcggtttctaaacgagttcggtttttggtttcttgattcgatttttggcttttgggttcaacccatggattttcgatttggttttggttttgataatattttttgtgcaatcgttcaaaaaatccgagttagattgggtttaaatgttatcaaaaccaaaaccgatggtagaaccccaaaactgaacccaatcgaaaatccatgggttgaacctcaaaaacgaacccaataaattctaaaaaccaaaatccaaaattagcggataaaaattttaaaatacatatctactggtaaggtgtttttgacaatttatgtaaattatagaaaaaaatgtattaagtgtttgctatttaagataaaaatggcgttaaaaatagaattggttagaaatgaaccatttttttatattttttacttcatttattaagtaaatagtagaggtgaattagagatgatctaagatattttttttctttcaaaaagagcttcgaaaacacttttccgacagaaacaagacacaaacctttttactcgtagaccgacgatttttttaaagatcaaagacctaagaagatttgtctcccatcttcttaaaacatatgtgagatagacacttctagcctcttctatgctaaaaaaaagagctcgcctttagtttggttaatttgagacaatggtctccacaaaaacataggtagttaggtactcacgcgccaagttactaatctcaaatcaaacataactgtatgatcgtcaacacattttacaagttaacaaaagagcattgctttagacttaaaaattgctttgagaaaaaacaaacacctaagaatttctaaaagcgatgatttcaactctctttaaaatgtatatatatgttacattttggtctttaaacttttcaagaaagtacaattcaactcctattttcaaaatgttcttacatgagcgatatgattttaccgtttaaaatgtaaaccatgataactatttctaaattccaatttttgtttcgttgatcaatctcattatcattcaatgcatacaaaacacaatacaatctgttttgtcatcttcttcaaggccgaaagacaactaaacatatgaagactgaagaggacaatctagcaaactcaaacttcactataaatttgcaattgctattggtagccatgaagtaacatttttgaggaggttaacaaaattttaacagtaaattcaataatccatcaccacgatatgtaatcatctacacaaacacagatgatgatagtcatatatgttcgtaatcacacaagcaagaaatcgcaatgtactttacaccgtcttctctgacttgaacacgggaattggttgtctaagaagtagattgtttcccacggatttaccaagtacatttaaatccttcaccctttgtatctctcttcctgttttttgttttgcaatcaacaaaaaaaaaagtaaaaatcccatcttaaattcacgcaaaaaaacacattgaatcaaccatcactatagtagtttcaagaaaaatctgcacttccataatttctttctattacaccattatactaggaaaacttcatagcaatattatctaaatacaaagcaatttttactagtaatattcgtagatttgtcaatatatagtgtcctaataaagaaaaaataacggaactataatgctctaataggtagattttttttagtataatgtcctaataagagaaaaaacaaatgtacaattatgtaataataagaaaagaaagtaatatgcttaatagacaaataaatgaaaccatgttgctatttcttgcatctagtccttatttgaataatatgtagcatattaactacttatgaattttatatataaatgataaaaaattccgtgag

SEQ ID NO: 11 - protein, 227 aa, Taraxacum officinale
MSTVKSKLSTPPTDHFSGNHQLLLITATAKTYIYNFHHNSVPYSHFNPKKATQTYTAMADNGNTGRQKDDDGGHDGGRPNPTTPPSPSRTPRRPRRTTSPPKHSPGASSSTMPAPPTPPAPTGITGASSSSLGTNIISFTPPKTKRTKSVICPICKKDMCHEKALCGHIRWHTQEERLAASIAIARALSSNVVVSGNGDEDEGPSKKYKLPDLNKSPPPEEEDEDAA

SEQ ID NO: 12 - DNA, 3217 bp, Taraxacum officinale
  source 1..3217: mol_type = unassigned DNA
gttgaaaggaaggatcggcagagaacaagattgagagagttttatcgaagagaagagatggttgtgttgattgacggcagagttcagcaatggcagatatgcgtgtttaaagcgcagtttgtggacagtttgtccaaaggccttcaagtgggagattgttgagatgaagacccttttgaccaagtacttaaatgtgattggttgcatgggaaagagctttacacttcattggttgaaaatgaagcatgttatttaatagtacgtctccacttgttctttatgaaggatgaagacagccaccaagtctttatgaatgtttcaaggagaagacaatgattagatgctgcctgctcccttacttcttcactataaataggtgcttaaggcacctctcttcttacaccaaacaacacacaagtcacagattacaaagattgagagagttttcattttctctctagagtttgttttgtcaaaagttttgtgtgcagattaattagttgagagacactcctaattaatataaccacttacacttgtcattgattagcattctagtgtcagagagttctgagtgattgtaacctttgtgtgatttatatataaatctctctcttgttcgccagtggacgtagctgattatcctgatcagtgaaccacttaaaatatcgtgttgatattatttatctgcagattattttaccagatagttttattccgcaccgcaaagcataccgtccattatttgtcagacgtacctactctcagctcatcccagcaggagttagacctgcaggcagccagccgtatttcacaacagtgaattatgcaacaaatggaaacacacaattatcgtagtacacatgtaattaacagggatcaatttccatttattgttccatcaattgccaaatttaaaagataacaaatgcatcctttttgaatggaaaagtatgatacataaaaccagagagaggtatacatttagtccagaaacggtggaaattactttacctagagttgcgtgaatcaaatggaaaatcagaggaagagaccctaaatcgcaatccatcttctattccctatagaatcaaatggaaatttcttacttccgacctttttcatttccacaagtttgtaatcgataagatcataaaagaaaaccgatttacattagcaaatctttaaggcagtttgtagttctcaagtatgcgttacgtatgaaattctagacagaaaagggacgacagggaaaataatggtggaataaataaacaatcaagaatcaaaagataataatccttaaaactaacaaaaattctatcaataagccctacaaatgagaaatatgacggtggagactcacctgattgatgcgagaaaaggaaagccgccgacatttgtgcttcttcccgcgttaagaaaaatcgcagatgacattacgattctttttctacagaggaataggggggttgcgactttgcgtgatttgttgttaatgagtgatgcgtcgtccgttttgatcggtagcccatggagactgatttatttgacagatagatgtcgttctcgttgatgatgcaggtgtaattggcggcgttggtgaagcgaagatggcatagggaatggatttgcagaggagaaaaggagtttcacaaatatggattattgaattgtcgtttcagagtcgcgatggttagtaatcgatggatttgggtttgcacctgttcaaggggtggtgacgtgtcttactgtaagaaagagtcgcgatggttagtaatcgacgatggatttgggtttgcaacatcgttgacggaaacgacgcggcgatggagagaggcggtagagggaatgaaacgggagatttaccttcaaccgcaaacatgtcttcgttcctcgaaaatcatatgcgccctttatgaaaattccttcacgcgccgtacacgcgctcagaaaaagcatagggttttcttatataataatgatataacttaattttaaataacatcgtactatacgtttcaaaataaattagagttttaataaaattacggaatgattaaacctattacaaacaaatataagtgtcggtaatataaaatttgtaacaacgaaaacatttcaaaaaatctgaatgcggaaatccataacatgagaatacgattcgtttgcagtcacgccggtttcttcaccttctgagggtcctgaaaaaggttaataaacaatgggtaagctaaagtttagggatcatacacattacttgtctattatatgagttagatacattcacatatcacatacatacaagcatacacatattacaaaaaatcctaacacatatggttggtaccaactactatttagtggaccaggttattcttgggggtcaattgtggttaaccataaatgtttggagtttaggttctctttgggggccgaccataaacacccatatcatatctctgtctaggttctctttggggactgactatagacaactttgctcagattctccttggaggataaccatcctcgacgagattaacaacggaccaccactgttttcagcttgtaacctgcaacatcaatagttgcgatcttcggcgacaatgatgaaccaacatcaacacaacagtagtgccggtatgttagtcgttggataatagaggcatcatctgacccttttagcggtgacttttgttgcctatgaaggtcttatcagttatatatcgattaggcttggttctaggttataagaaaaaatgattctctatccggtatgacatagagacaaaccggtctacttttataagaaaatcatactcgtatttgaaacgaaaatcatatttgaaacaatgtcgtgttaataattaatatgtgtatgtttaaatttagaatatcctgaacttatatttagttcgcaaccctatgtaagatcgaactaattttaaaccgtaaccagacatgcatgtttgatcagtaataaaaaggctatcatcgtcgaaaatattaaaacaaaatataatttcttatttggcgccatttcatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcg

SEQ ID NO: 13 - DNA, 684 bp, Taraxacum officinale
  source 1..684: mol_type = unassigned DNA
atgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatggcagataatggcaacaccggccgtcaaaaggatgacgacggtggccatgatgggggacgcccaaatccgaccaccccaccctccccttcccgcaccccgcgaagacccaggcggaccacatcgccgcctaaacattctccgggggcgtcttcaagcaccatgccagcgccgcctactccccctgcgccgacgggaatcaccggtgcgtctagttcttctctgggtactaatataatttcatttactccacccaaaaccaaaagaacgaagtcggtgatttgtccgatctgtaagaaagatatgtgccatgagaaggcgctgtgtggccacatccggtggcatactcaggaagaaagattggcggccagcattgctatagcaagagcgctatcttctaacgttgttgtttctggcaatggcgatgaagatgaaggtccatctaaaaagtataaactcccggacctgaacaagtcaccaccgccggaggaggaggacgaggacgctgcctga

SEQ ID NO: 14 - DNA, 3925 bp, Taraxacum officinale
  source 1..3925: mol_type = unassigned DNA
ttttcttgcgggagaggcatgtatgtgtggcctttttgcaataatactatgcatgggagatgcataaagtttatctttattttgttttagtttaataatgtgtggcaaactttatatcatttatgatttcggccatgtactgctacatcgttctaggatggttattccctagaatttgcttttatatattttttttcttcgttaattcttctcttcctttaacgttatttcatgtgaatttgcgatatcatgatattgcaggaacgtaacttagaactcaacgataaaaagtgtgttgagatcgtggaagttgatcgtttatcttgaattcacaagataagcaacatgagatagaataattaattactgcggagatttctttttagattcatcttattcgttgtaaattgcaatgcaaatttttaagtagaataacgaaagcgtgttgttatttacggttaaatgcaaaaaatcaataacatatctccataattgatttgtttagtatgcattctcatgtgttctattgcaaaattctattttgaaattcaattaaagcaatgcattttagatataaagcaattccatcacttaattatattgggtagatttttaccaaatcactgtcctaaaagaaaaataaaaaatgaaagttcaatgccatagtgatcccgtcaattatgtgatcaatttttaccagagcaaaaccccaaaagttaaagagtataattgcagaacgacaacaatctgaggatgatactaactccggtataaaccgaatgcttttgtggggatttcactaatcaactatttggctctcatcacaaggccgtgttaatggattactattagttataaggccatcccaagttgtttggctatattattaccttttaaaaaccaaaatgtgtaaactgtaaaattacacttttggttcttgtattttctaatttttagaattatgtatttggttcttgtagtttttttatttgtatgaatcatattagttttggtccacgataacttttattttctataattacacttttggtcattgtagttttaatttgcaagaaaaaaataatctatctttttagtccatatatatagtttgcaatattctatattttaggttcaggttatatatcttttacaaaaatactacttatgattttttaatgaatatatcatatttcgtaaattacataaacattttatataattttatttagtcagaactataggattcgcatgataactttgctgaacttaattgggcttcatttttaacaaaggataatgcatcatgcatgacataaaaatttgtaatgtttctaatgtgcatgcactacaagaacctgacccttcatatacattaatgaatgattctcttttcttattatatcttagcaaacggtttggataagagtctgaacaatattaagaattgcactagctagctaggggagtccatgcagcaagcaaattaattagaggactttttaagaggcttgatcacttcattgtatatctaatggcactgtggatatatgattggattgtgtgtatgtatggaatttaatattactaaaaaatatacatttaatgacaaacaataacacactaatttataacttacaaaagcgtgtatcattaaagaaaatttcatgtttcttaaaatatggagaatttactacttacataattatgtgtcataaatttatgtcatcttgtgtgatatttatgacttacaaaaatgtatgtgatatttatgacttacaaaaatgtatgttatgtttatcgtcataaaatctgtaaaaaaaaatagaaacatggaggatactttcaatgtgtagaaacttgacagcataaacaggtttattaccttcatcaaaacttatcttacttcaatctcttggagatgcaggttgattttaatgctaaatgaacttttcaaatgtataaatatatatcttttgacggatttgacattgatggagatgaagatacaccagtgaattcacctttcaatgatcatactgacataccaagtactcttatcccctctccacctccacagaacgaacctcaatttcaaaaacgtaactgccgctgttctaaaaacagaaaattttcaaaaacgtaactatcatttaaatatttaatcatttttcaattggattcatgctgtaatagaagttactggactttaaacttcttgtagctgatgagattcatgaacaacagtccaaggagattttccaaatcaacaagtttctgatgagaacaaggtaaaaaaaataagtttttcatttaaacatatgattttggttttcaaaagataattaattataggaactcttctgaaaagtcccaaagtttacccgtaaaattgattttggttttttttttttaaattgaataaaaaagtccataaaattggataaattattcgatttagcccaatttacctagtatatctggtaaaaatgggttaaatcgaataatttaggcagtttgttgggcttttttgcgcaaaaacattattttgggacttaatcgttaattttccccaagtttcagtttatttacagtattggaataaaaccgaacctagtgcgctaattttgtcttttggtgtttagaattatttgggtttattggtctcggtttctaaacgagttcggtttttggtttcttgattcgatttttggcttttgggttcaacccatggattttcgatttggttttggttttgataatattttttgtgcaatcgttcaaaaaatccgagttagattgggtttaaatgttatcaaaaccaaaaccgatggtagaaccccaaaactgaacccaatcgaaaatccatgggttgaacctcaaaaacgaacccaataaattctaaaaaccaaaatccaaaattagcggataaaaattttaaaatacatatctactggtaaggtgtttttgacaatttatgtaaattatagaaaaaaatgtattaagtgtttgctatttaagataaaaatggcgttaaaaatacaattggttagaaatgaaccatttttttgttgtaaatgagtggtgcatcaacgatggtggatgccatcttttccttccaccatatacatctttgtcaaacaccatacacacaaattctcatatgtcaaagatattaattatcatgtccactgagtttgtaatataaatagatgatcaattacaacgagaaatatacactgaattctcatttctaaattgaagaatctctctctctctctctcatctctcagttttcaaagttttataagttactagtttatagtgtttctaattacttcatatttataacacgttatcagcacgagagctctaagcaagatatgcaattacttaataaagtttataaaaataaacaaatcataataaacaacatagatataaaattaattattaccatggacatgatggactactaacattttatttcttatactaacatttatttatgttaactaacatttatttatgtttctaacatttatttatgcatactaacatttatttatgttaactaacatttatttatgtttctaacatttatttatgcatactaacatttatttatgttactaacttttatttatgtatactaatgtttatttatcaaatttatttaacatttactattttttgattatattttttaattagccgatagaatttatttatttaattattctatttattaaacttatttttataaatatatatatatatataaccgttatatatataacggctatatatataacgtctatatatatataacggctatatttttacactataaataaacacatcattctccatttttataccacaacaaataatttctctcatcttccaaatcatcaagagaaaa

SEQ ID NO: 15 - DNA, 7826 bp, Taraxacum officinale
  source 1..7826: mol_type = unassigned DNA
gttgaaaggaaggatcggcagagaacaagattgagagagttttatcgaagagaagagatggttgtgttgattgacggcagagttcagcaatggcagatatgcgtgtttaaagcgcagtttgtggacagtttgtccaaaggccttcaagtgggagattgttgagatgaagacccttttgaccaagtacttaaatgtgattggttgcatgggaaagagctttacacttcattggttgaaaatgaagcatgttatttaatagtacgtctccacttgttctttatgaaggatgaagacagccaccaagtctttatgaatgtttcaaggagaagacaatgattagatgctgcctgctcccttacttcttcactataaataggtgcttaaggcacctctcttcttacaccaaacaacacacaagtcacagattacaaagattgagagagttttcattttctctctagagtttgttttgtcaaaagttttgtgtgcagattaattagttgagagacactcctaattaatataaccacttacacttgtcattgattagcattctagtgtcagagagttctgagtgattgtaacctttgtgtgatttatatataaatctctctcttgttcgccagtggacgtagctgattatcctgatcagtgaaccacttaaaatatcgtgttgatattatttatctgcagattattttaccagatagttttattccgcaccgcaaagcataccgtccattatttgtcagacgtacctactctcagctcatcccagcaggagttagacctgcaggcagccagccgtatttcacaacagtgaattatgcaacaaatggaaacacacaattatcgtagtacacatgtaattaacagggatcaatttccatttattgttccatcaattgccaaatttaaaagataacaaatgcatcctttttgaatggaaaagtatgatacataaaaccagagagaggtatacatttagtccagaaacggtggaaattactttacctagagttgcgtgaatcaaatggaaaatcagaggaagagaccctaaatcgcaatccatcttctattccctatagaatcaaatggaaatttcttacttccgacctttttcatttccacaagtttgtaatcgataagatcataaaagaaaaccgatttacattagcaaatctttaaggcagtttgtagttctcaagtatgcgttacgtatgaaattctagacagaaaagggacgacagggaaaataatggtggaataaataaacaatcaagaatcaaaagataataatccttaaaactaacaaaaattctatcaataagccctacaaatgagaaatatgacggtggagactcacctgattgatgcgagaaaaggaaagccgccgacatttgtgcttcttcccgcgttaagaaaaatcgcagatgacattacgattctttttctacagaggaataggggggttgcgactttgcgtgatttgttgttaatgagtgatgcgtcgtccgttttgatcggtagcccatggagactgatttatttgacagatagatgtcgttctcgttgatgatgcaggtgtaattggcggcgttggtgaagcgaagatggcatagggaatggatttgcagaggagaaaaggagtttcacaaatatggattattgaattgtcgtttcagagtcgcgatggttagtaatcgatggatttgggtttgcacctgttcaaggggtggtgacgtgtcttactgtaagaaagagtcgcgatggttagtaatcgacgatggatttgggtttgcaacatcgttgacggaaacgacgcggcgatggagagaggcggtagagggaatgaaacgggagatttaccttcaaccgcaaacatgtcttcgttcctcgaaaatcatatgcgccctttatgaaaattccttcacgcgccgtacacgcgctcagaaaaagcatagggttttcttatataataatgatataacttaattttaaataacatcgtactatacgtttcaaaataaattagagttttaataaaattacggaatgattaaacctattacaaacaaatataagtgtcggtaatataaaatttgtaacaacgaaaacatttcaaaaaatctgaatgcggaaatccataacatgagaatacgattcgtttgcagtcacgccggtttcttcaccttctgagggtcctgaaaaaggttaataaacaatgggtaagctaaagtttagggatcatacacattacttgtctattatatgagttagatacattcacatatcacatacatacaagcatacacatattacaaaaaatcctaacacatatggttggtaccaactactatttagtggaccaggttattcttgggggtcaattgtggttaaccataaatgtttggagtttaggttctctttgggggccgaccataaacacccatatcatatctctgtctaggttctctttggggactgactatagacaactttgctcagattctccttggaggataaccatcctcgacgagattaacaacggaccaccactgttttcagcttgtaacctgcaacatcaatagttgcgatcttcggcgacaatgatgaaccaacatcaacacaacagtagtgccggtatgttagtcgttggataatagaggcatcatctgacccttttagcggtgacttttgttgcctatgaaggtcttatcagttatatatcgattaggcttggttctaggttataagaaaaaatgattctctatccggtatgacatagagacaaaccggtctacttttataagaaaatcatactcgtatttgaaacgaaaatcatatttgaaacaatgtcgtgttaataattaatatgtgtatgtttaaatttagaatatcctgaacttatatttagttcgcaaccctatgtaagatcgaactaattttaaaccgtaaccagacatgcatgtttgatcagtaataaaaaggctatcatcgtcgaaaatattaaaacaaaatataatttcttatttggcgccatttcatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatggcagataatggcaacaccggccgtcaaaaggatgacgacggtggccatgatgggggacgcccaaatccgaccaccccaccctccccttcccgcaccccgcgaagacccaggcggaccacatcgccgcctaaacattctccgggggcgtcttcaagcaccatgccagcgccgcctactccccctgcgccgacgggaatcaccggtgcgtctagttcttctctgggtactaatataatttcatttactccacccaaaaccaaaagaacgaagtcggtgatttgtccgatctgtaagaaagatatgtgccatgagaaggcgctgtgtggccacatccggtggcatactcaggaagaaagattggcggccagcattgctatagcaagagcgctatcttctaacgttgttgtttctggcaatggcgatgaagatgaaggtccatctaaaaagtataaactcccggacctgaacaagtcaccaccgccggaggaggaggacgaggacgctgcctgattttcttgcgggagaggcatgtatgtgtggcctttttgcaataatactatgcatgggagatgcataaagtttatctttattttgttttagtttaataatgtgtggcaaactttatatcatttatgatttcggccatgtactgctacatcgttctaggatggttattccctagaatttgcttttatatattttttttcttcgttaattcttctcttcctttaacgttatttcatgtgaatttgcgatatcatgatattgcaggaacgtaacttagaactcaacgataaaaagtgtgttgagatcgtggaagttgatcgtttatcttgaattcacaagataagcaacatgagatagaataattaattactgcggagatttctttttagattcatcttattcgttgtaaattgcaatgcaaatttttaagtagaataacgaaagcgtgttgttatttacggttaaatgcaaaaaatcaataacatatctccataattgatttgtttagtatgcattctcatgtgttctattgcaaaattctattttgaaattcaattaaagcaatgcattttagatataaagcaattccatcacttaattatattgggtagatttttaccaaatcactgtcctaaaagaaaaataaaaaatgaaagttcaatgccatagtgatcccgtcaattatgtgatcaatttttaccagagcaaaaccccaaaagttaaagagtataattgcagaacgacaacaatctgaggatgatactaactccggtataaaccgaatgcttttgtggggatttcactaatcaactatttggctctcatcacaaggccgtgttaatggattactattagttataaggccatcccaagttgtttggctatattattaccttttaaaaaccaaaatgtgtaaactgtaaaattacacttttggttcttgtattttctaatttttagaattatgtatttggttcttgtagtttttttatttgtatgaatcatattagttttggtccacgataacttttattttctataattacacttttggtcattgtagttttaatttgcaagaaaaaaataatctatctttttagtccatatatatagtttgcaatattctatattttaggttcaggttatatatcttttacaaaaatactacttatgattttttaatgaatatatcatatttcgtaaattacataaacattttatataattttatttagtcagaactataggattcgcatgataactttgctgaacttaattgggcttcatttttaacaaaggataatgcatcatgcatgacataaaaatttgtaatgtttctaatgtgcatgcactacaagaacctgacccttcatatacattaatgaatgattctcttttcttattatatcttagcaaacggtttggataagagtctgaacaatattaagaattgcactagctagctaggggagtccatgcagcaagcaaattaattagaggactttttaagaggcttgatcacttcattgtatatctaatggcactgtggatatatgattggattgtgtgtatgtatggaatttaatattactaaaaaatatacatttaatgacaaacaataacacactaatttataacttacaaaagcgtgtatcattaaagaaaatttcatgtttcttaaaatatggagaatttactacttacataattatgtgtcataaatttatgtcatcttgtgtgatatttatgacttacaaaaatgtatgtgatatttatgacttacaaaaatgtatgttatgtttatcgtcataaaatctgtaaaaaaaaatagaaacatggaggatactttcaatgtgtagaaacttgacagcataaacaggtttattaccttcatcaaaacttatcttacttcaatctcttggagatgcaggttgattttaatgctaaatgaacttttcaaatgtataaatatatatcttttgacggatttgacattgatggagatgaagatacaccagtgaattcacctttcaatgatcatactgacataccaagtactcttatcccctctccacctccacagaacgaacctcaatttcaaaaacgtaactgccgctgttctaaaaacagaaaattttcaaaaacgtaactatcatttaaatatttaatcatttttcaattggattcatgctgtaatagaagttactggactttaaacttcttgtagctgatgagattcatgaacaacagtccaaggagattttccaaatcaacaagtttctgatgagaacaaggtaaaaaaaataagtttttcatttaaacatatgattttggttttcaaaagataattaattataggaactcttctgaaaagtcccaaagtttacccgtaaaattgattttggttttttttttttaaattgaataaaaaagtccataaaattggataaattattcgatttagcccaatttacctagtatatctggtaaaaatgggttaaatcgaataatttaggcagtttgttgggcttttttgcgcaaaaacattattttgggacttaatcgttaattttccccaagtttcagtttatttacagtattggaataaaaccgaacctagtgcgctaattttgtcttttggtgtttagaattatttgggtttattggtctcggtttctaaacgagttcggtttttggtttcttgattcgatttttggcttttgggttcaacccatggattttcgatttggttttggttttgataatattttttgtgcaatcgttcaaaaaatccgagttagattgggtttaaatgttatcaaaaccaaaaccgatggtagaaccccaaaactgaacccaatcgaaaatccatgggttgaacctcaaaaacgaacccaataaattctaaaaaccaaaatccaaaattagcggataaaaattttaaaatacatatctactggtaaggtgtttttgacaatttatgtaaattatagaaaaaaatgtattaagtgtttgctatttaagataaaaatggcgttaaaaatacaattggttagaaatgaaccatttttttgttgtaaatgagtggtgcatcaacgatggtggatgccatcttttccttccaccatatacatctttgtcaaacaccatacacacaaattctcatatgtcaaagatattaattatcatgtccactgagtttgtaatataaatagatgatcaattacaacgagaaatatacactgaattctcatttctaaattgaagaatctctctctctctctctcatctctcagttttcaaagttttataagttactagtttatagtgtttctaattacttcatatttataacacgttatcagcacgagagctctaagcaagatatgcaattacttaataaagtttataaaaataaacaaatcataataaacaacatagatataaaattaattattaccatggacatgatggactactaacattttatttcttatactaacatttatttatgttaactaacatttatttatgtttctaacatttatttatgcatactaacatttatttatgttaactaacatttatttatgtttctaacatttatttatgcatactaacatttatttatgttactaacttttatttatgtatactaatgtttatttatcaaatttatttaacatttactattttttgattatattttttaattagccgatagaatttatttatttaattattctatttattaaacttatttttataaatatatatatatatataaccgttatatatataacggctatatatataacgtctatatatatataacggctatatttttacactataaataaacacatcattctccatttttataccacaacaaataatttctctcatcttccaaatcatcaagagaaaa

SEQ ID NO: 16 - DNA, 1547 bp, Lactuca sativa
  source 1..1547: mol_type = unassigned DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaaaccagctctgtatatattggttatgttatgggttatatagagttgtttaaaaggcagttatcgattacgttctcagttctagagttttcacaaaaatcaattctcctgtatcacatcgaaagcaaaccggtctatttataaaagagatacatatttgaaacaacatattttttaattatatttatgtgttgtttataaatctagaaacccgtaaatttatatttagttcagtatccaatataggatctgaaactactttataaccgttaccagacaaataccggtcaagtttatattggtgtagttttcgattcctttgattttatatctttttggcgccatttcacgtcaacggtttaaaaacaagttatcgtttctccggtacagttaaccaccaaccgccacaaataaccgtcaccggaatacctataaatacaacttccatttcaatcgtctctctctgcactcccattctttgaatccaaaaaaggctacacacacacacagacaaca

SEQ ID NO: 17 - DNA, 1532 bp, synthetic construct
  misc_feature 1..1532: note = Lactuca sativa LSAT_8X112340 promoter without 2 MYB binding sites
  source 1..1532: mol_type = other DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaaaccagctctgtatatattggttatgttatgggttatatagagttgtttaaaaggcagttatcgattacgttctcagttctagagttttcacaaaaatcaattctcctgtatcacatcgaaagcaaaccggtctatttataaaagagatacatatttgaaacaacatattttttaattatatttatgtgttgtttataaatctagaaacccgtaaatttatatttagttcagtatccaatataggatctgaaactactttataaccgttaccagacaaataccggtcaagtttatattggtgtagttttcgattcctttgattttatatctttttggcgccatttcacgtcaacggtttaaaaacaagttatcgtttctccggtacagttaaccacccaaataccggaatacctataaatacaacttccatttcaatcgtctctctctgcactcccattctttgaatccaaaaaaggctacacacacacacagacaaca

SEQ ID NO: 18 - DNA, 2882 bp, synthetic construct
  misc_feature 1..2882: note = Lactuca sativa LSAT_8X112340 promoter with Taraxacum officinale Par allele MITE insertion
  source 1..2882: mol_type = other DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaaaccagctctgtatatattggttatgttatgggttatatagagttgtttaaaaggcagttatcgattacgttctcagttctagagttttcacaaaaatcaattctcctgtatcacatcgaaagcaaaccggtctatttataaaagagatacatatttgaaacaacatattttttaattatatttatgtgttgtttataaatctagaaacccgtaaatttatatttagttcagtatccaatataggatctgaaactactttataaccgttaccagacaaataccggtcaagtttatattggtgtagttttcgattcctttgattttatatctttttggcgccatttcacgtcaacggtttaaaaacaagttatcgtttctccggtacagttaaccaccactgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctacaaccgccacaaataaccgtcaccggaatacctataaatacaacttccatttcaatcgtctctctctgcactcccattctttgaatccaaaaaaggctacacacacacacagacaaca

SEQ ID NO: 19 - DNA, 20 bp, synthetic construct
  misc_feature 1..20: note = target specific sequence of RNA-1
  source 1..20: mol_type = other DNA
catccggtggcatacacagg

SEQ ID NO: 20 - DNA, 1148 bp, synthetic construct
  misc_feature 1..1148: note = Lactuca sativa LSAT_8X112340 promoter with deletion
  source 1..1148: mol_type = other DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaccgccacaaataaccgtcaccggaatacctataaatacaacttccatttcaatcgtctctctctgcactcccattctttgaatccaaaaaaggctacacacacacacagacaaca

SEQ ID NO: 21 - protein, 89 aa, Helianthus annuus
MSSPSKNKSKSSICPVCKRDLCHEKALNGHIRWHTQQEREAAGIGNAKAVASASVVIRDANVRRPETSKPVKLPDLNKSPPREEDKDAA

SEQ ID NO: 22 - protein, 157 aa, Lactuca sativa
MADDGNTARQQADAGGHGNARPNSSTPPSSPSQPPRRPRRAGATTPSKLSQAASSSTNLPPPPTPTPTPPTPSADGILLGTARRPVICPICKKDMYHEKALCGHIRWHTQEERLAASRDIARALSANVVSGQRGDGEQGPSKRFKLPDLNEPPPSED

SEQ ID NO: 23 - DNA, 36 bp, Taraxacum officinale
  source 1..36: mol_type = unassigned DNA
tgtggccacatccggtggcatacacaggaggaaaga

SEQ ID NO: 24 - DNA, 37 bp, synthetic construct
  misc_feature 1..37: note = pKG10821-1
  source 1..37: mol_type = other DNA
tgtggccacatccggtggcatacaccaggaggaaaga

SEQ ID NO: 25 - DNA, 37 bp, synthetic construct
  misc_feature 1..37: note = pKG10821-4
  source 1..37: mol_type = other DNA
tgtggccacatccggtggcatacacaaggaggaaaga

SEQ ID NO: 26 - DNA, 35 bp, synthetic construct
  misc_feature 1..35: note = pKG10821-5
  source 1..35: mol_type = other DNA
tgtggccacatccggtggcatacaaggaggaaaga

SEQ ID NO: 27 - DNA, 33 bp, synthetic construct
  misc_feature 1..33: note = pKG10821-6
  source 1..33: mol_type = other DNA
tgtggccacatccggtggcataaggaggaaaga

SEQ ID NO: 28 - protein, 12 aa, synthetic construct
  REGION 1..12: note = pKG10821-1
CGHIRWHTPGGK

SEQ ID NO: 29 - protein, 12 aa, synthetic construct
  REGION 1..12: note = pKG108121-4
CGHIRWHTQGGK

SEQ ID NO: 30 - protein, 11 aa, synthetic construct
  REGION 1..11: note = pKG10821-5
CGHIRWHTRRK

SEQ ID NO: 31 - protein, 11 aa, synthetic construct
  REGION 1..11: note = pKG10821-6
CGHIRWHKEER

SEQ ID NO: 32 - protein, 12 aa, Taraxacum officinale
CGHIRWHTQEER

SEQ ID NO: 33 - DNA, 474 bp, synthetic construct
  misc_feature 1..474: note = Lactuca sativa LSAT_8X112340 coding sequence
  source 1..474: mol_type = other DNA
atggcagatgatggcaacaccgcccgtcaacaggctgacgccggtggccatggaaacgcacgtccaaactcatccacccccccttcgtcaccctcccaacctccaagaagaccacgccgtgccggagccactacaccgtcaaagctttcccaggcggcatcttcgtcaacaaatcttccaccgccgccaacgccaacaccaactccgcccaccccttctgccgacgggatattactaggtactgcaagaaggccggtaatatgcccgatttgcaaaaaggatatgtatcatgaaaaggcgctgtgtggccacatccggtggcacacgcaggaggagagactggcggcaagcagggatattgcccgggcgctttctgccaacgttgtttctgggcaaagaggcgatggagaacaagggccgtctaaacggttcaaactgccggatcttaacgagccaccaccgtcggaggactga

SEQ ID NO: 34 - DNA, 3021 bp, synthetic construct
  misc_feature 1..3021: note = LsPromtoter_LsCoding sequence_To3'UTR
  source 1..3021: mol_type = other DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaaaccagctctgtatatattggttatgttatgggttatatagagttgtttaaaaggcagttatcgattacgttctcagttctagagttttcacaaaaatcaattctcctgtatcacatcgaaagcaaaccggtctatttataaaagagatacatatttgaaacaacatattttttaattatatttatgtgttgtttataaatctagaaacccgtaaatttatatttagttcagtatccaatataggatctgaaactactttataaccgttaccagacaaataccggtcaagtttatattggtgtagttttcgattcctttgattttatatctttttggcgccatttcacgtcaacggtttaaaaacaagttatcgtttctccggtacagttaaccaccaaccgccacaaataaccgtcaccggaatacctataaatacaacttccatttcaatcgtctctctctgcactcccattctttgaatccaaaaaaggctacacacacacacagacaacaatggcagatgatggcaacaccgcccgtcaacaggctgacgccggtggccatggaaacgcacgtccaaactcatccacccccccttcgtcaccctcccaacctccaagaagaccacgccgtgccggagccactacaccgtcaaagctttcccaggcggcatcttcgtcaacaaatcttccaccgccgccaacgccaacaccaactccgcccaccccttctgccgacgggatattactaggtactgcaagaaggccggtaatatgcccgatttgcaaaaaggatatgtatcatgaaaaggcgctgtgtggccacatccggtggcacacgcaggaggagagactggcggcaagcagggatattgcccgggcgctttctgccaacgttgtttctgggcaaagaggcgatggagaacaagggccgtctaaacggttcaaactgccggatcttaacgagccaccaccgtcggaggactgattttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcatgatattgcaggaatgtaacttagaactcaacgataaaaagtgtgttgagatcgtggaagttgatcatttatcttgaattcataagataagcaacatgagataggataattaattactgtggggatttctttttatattcatattattcgttgtaatgcaaatttttaagtagaataacgaatgtgtcttattatttacggttaaatgcaaaagatcattaacatatctccattgatttgtttactgatgtattctcatgtgttctgttgctaaattctattttgaaaagtatattcaattaaagcaatgcattttagatataaagcaactccataacttataattgtgtagatttttaccaattcaatgtcttaaaagaaaaataaaaattgaaagtttaattaattgccatagtgatcccgttcccgtcaattatgtgatcaatttttaccagagcaaaaccccacatttcttgtagtgaacaatctgtggatgataaccgatcgaagacttttgtggggatttcactaaccaactatttggctctcattaattactattagttatataaggccatcccaagtattttggcaatattcaaaacgagtaaactataaaattacacttttggttcttgtatttctaacttttagaattatgtatttggttcttatagttttttatttgtatgaattacattacttttggtccacaatattttttattttctataattccatttttagtc

SEQ ID NO: 35 - DNA, 3006 bp, synthetic construct
  misc_feature 1..3006: note = LsPromoter-without-MYB-sites_LsCoding sequence_To3'UTR
  source 1..3006: mol_type = other DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaaaccagctctgtatatattggttatgttatgggttatatagagttgtttaaaaggcagttatcgattacgttctcagttctagagttttcacaaaaatcaattctcctgtatcacatcgaaagcaaaccggtctatttataaaagagatacatatttgaaacaacatattttttaattatatttatgtgttgtttataaatctagaaacccgtaaatttatatttagttcagtatccaatataggatctgaaactactttataaccgttaccagacaaataccggtcaagtttatattggtgtagttttcgattcctttgattttatatctttttggcgccatttcacgtcaacggtttaaaaacaagttatcgtttctccggtacagttaaccacccaaataccggaatacctataaatacaacttccatttcaatcgtctctctctgcactcccattctttgaatccaaaaaaggctacacacacacacagacaacaatggcagatgatggcaacaccgcccgtcaacaggctgacgccggtggccatggaaacgcacgtccaaactcatccacccccccttcgtcaccctcccaacctccaagaagaccacgccgtgccggagccactacaccgtcaaagctttcccaggcggcatcttcgtcaacaaatcttccaccgccgccaacgccaacaccaactccgcccaccccttctgccgacgggatattactaggtactgcaagaaggccggtaatatgcccgatttgcaaaaaggatatgtatcatgaaaaggcgctgtgtggccacatccggtggcacacgcaggaggagagactggcggcaagcagggatattgcccgggcgctttctgccaacgttgtttctgggcaaagaggcgatggagaacaagggccgtctaaacggttcaaactgccggatcttaacgagccaccaccgtcggaggactgattttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcatgatattgcaggaatgtaacttagaactcaacgataaaaagtgtgttgagatcgtggaagttgatcatttatcttgaattcataagataagcaacatgagataggataattaattactgtggggatttctttttatattcatattattcgttgtaatgcaaatttttaagtagaataacgaatgtgtcttattatttacggttaaatgcaaaagatcattaacatatctccattgatttgtttactgatgtattctcatgtgttctgttgctaaattctattttgaaaagtatattcaattaaagcaatgcattttagatataaagcaactccataacttataattgtgtagatttttaccaattcaatgtcttaaaagaaaaataaaaattgaaagtttaattaattgccatagtgatcccgttcccgtcaattatgtgatcaatttttaccagagcaaaaccccacatttcttgtagtgaacaatctgtggatgataaccgatcgaagacttttgtggggatttcactaaccaactatttggctctcattaattactattagttatataaggccatcccaagtattttggcaatattcaaaacgagtaaactataaaattacacttttggttcttgtatttctaacttttagaattatgtatttggttcttatagttttttatttgtatgaattacattacttttggtccacaatattttttattttctataattccatttttagtc

SEQ ID NO: 36 - DNA, 4356 bp, synthetic construct
  misc_feature 1..4356: note = LsPromoter-with-MITE-insert_LsCoding sequence_To3'UTR
  source 1..4356: mol_type = other DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaaaccagctctgtatatattggttatgttatgggttatatagagttgtttaaaaggcagttatcgattacgttctcagttctagagttttcacaaaaatcaattctcctgtatcacatcgaaagcaaaccggtctatttataaaagagatacatatttgaaacaacatattttttaattatatttatgtgttgtttataaatctagaaacccgtaaatttatatttagttcagtatccaatataggatctgaaactactttataaccgttaccagacaaataccggtcaagtttatattggtgtagttttcgattcctttgattttatatctttttggcgccatttcacgtcaacggtttaaaaacaagttatcgtttctccggtacagttaaccaccactgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctacaaccgccacaaataaccgtcaccggaatacctataaatacaacttccatttcaatcgtctctctctgcactcccattctttgaatccaaaaaaggctacacacacacacagacaacaatggcagatgatggcaacaccgcccgtcaacaggctgacgccggtggccatggaaacgcacgtccaaactcatccacccccccttcgtcaccctcccaacctccaagaagaccacgccgtgccggagccactacaccgtcaaagctttcccaggcggcatcttcgtcaacaaatcttccaccgccgccaacgccaacaccaactccgcccaccccttctgccgacgggatattactaggtactgcaagaaggccggtaatatgcccgatttgcaaaaaggatatgtatcatgaaaaggcgctgtgtggccacatccggtggcacacgcaggaggagagactggcggcaagcagggatattgcccgggcgctttctgccaacgttgtttctgggcaaagaggcgatggagaacaagggccgtctaaacggttcaaactgccggatcttaacgagccaccaccgtcggaggactgattttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcatgatattgcaggaatgtaacttagaactcaacgataaaaagtgtgttgagatcgtggaagttgatcatttatcttgaattcataagataagcaacatgagataggataattaattactgtggggatttctttttatattcatattattcgttgtaatgcaaatttttaagtagaataacgaatgtgtcttattatttacggttaaatgcaaaagatcattaacatatctccattgatttgtttactgatgtattctcatgtgttctgttgctaaattctattttgaaaagtatattcaattaaagcaatgcattttagatataaagcaactccataacttataattgtgtagatttttaccaattcaatgtcttaaaagaaaaataaaaattgaaagtttaattaattgccatagtgatcccgttcccgtcaattatgtgatcaatttttaccagagcaaaaccccacatttcttgtagtgaacaatctgtggatgataaccgatcgaagacttttgtggggatttcactaaccaactatttggctctcattaattactattagttatataaggccatcccaagtattttggcaatattcaaaacgagtaaactataaaattacacttttggttcttgtatttctaacttttagaattatgtatttggttcttatagttttttatttgtatgaattacattacttttggtccacaatattttttattttctataattccatttttagtc

SEQ ID NO: 37 - protein, 21 aa, synthetic construct
  REGION 1..21: note = K2-2 like zinc finger domain
  VARIANT 2..3: note = misc_feature - Xaa can be any naturally occurring amino acid
  VARIANT 5..11: note = misc_feature - Xaa can be any naturally occurring amino acid
  VARIANT 12: note = MISC_FEATURE - Xaa can be K or R
  VARIANT 14..15: note = misc_feature - Xaa can be any naturally occurring amino acid
  VARIANT 18: note = misc_feature - Xaa can be any naturally occurring amino acid
  VARIANT 19: note = MISC_FEATURE - Xaa can be R or N
  VARIANT 20: note = misc_feature - Xaa can be any naturally occurring amino acid
CXXCXXXXXXXXAXXGHXXXH

SEQ ID NO: 38 - protein, 21 aa, synthetic construct
  REGION 1..21: note = consensus zinc finger domain
  VARIANT 2..3: note = misc_feature - Xaa can be any naturally occurring amino acid
  VARIANT 5..16: note = misc_feature - Xaa can be any naturally occurring amino acid
  VARIANT 18..20: note = misc_feature - Xaa can be any naturally occurring amino acid
CXXCXXXXXXXXXXXXHXXXH

SEQ ID NO: 39 - protein, 163 aa, Cichorium endivia
MVDDGTTARQQVDDGGHGNPRPNPTTPPPSPSRTPRRTRRAGATTPSKLSPAVSSTTLPPPPTPPTPPADRLVTGTSSSLGTQRVRRSKSVICPICKKDMCHEKALCGHIRWHTHEERQAASSDIARALSSNFSSGHGGEEQGPSKRFKVPDLNKPPPPEEDD

SEQ ID NO: 40 - protein, 161 aa, Hieracium aurantiacum
MVDDGTAARQQVDDGGHGERRPTPTTPPPSPSRTPRRSHRPGTTPPSKLSPAASSTALPPPPTPPTPTVVAGSSSSLTTPRIKRSKSVICPICKKDMCHEKALCGHIRWHTQEERLAASGDIARALSANFVSGGNGDEEQGSSKRFKVPDLNKPPPPEEDD

SEQ ID NO: 41 - protein, 166 aa, unidentified
  source 1..166: note = Senecio cambrensis
MVNSGDASRSDDNPNTPNRRAGKAHAAPSTSPSSPPPPIGRLSATTPLSGSKRPHSPTKKSNFICPICGKDLYHSKALNGHIRWHSQMERELKKKALRDIRERDVAVVAAADAVQVAPVPATVPVPVPTIVRTRLFHEQPQPQPQEEVPYNYAYLLPDLNASADEE

SEQ ID NO: 42 - protein, 203 aa, Hevea brasiliensis
MVNSSPSSSSSSSSPTPPPEVQVGPGGSSVGGESEEGCSSRKKKAGSNDQQGDEEGVQKRLKRGEMDSPISEPVCCICNKKFGSWKGVFGHMRAHPEREWRGAFPPPKEKAGAININHQQILQQQLAPTLLSLGREALARMTNHHGSGAANAAAPSRRAGDTELNREQQELAGPSTIIHPSLRGFDLNLPPPPEEANEDDRNN

SEQ ID NO: 43 - protein, 230 aa, Cucurbita moschata
MEKNANTNVPSETSSDQRHGGERSPTGPTSPPPTHNRDNNPNPTTEHSSYSDTLRVPPAAETSRRDALLDVGTSYQNIVGSSEGEPGKKRGRGDGEEEEEQQQPPQQQVKAAKKKGELTEVPKGEPRCATCNKVFKSWKALFGHLRSHPERTYRGALPPPTAAELDIRRCQQQLASTLLTVAQEVAASRRGLDIDLNQPSTADDGDSPEKTGGVGFDLNADPPPESDDED

SEQ ID NO: 44 - protein, 242 aa, Eutrema salsugineum
MVTEEEINEISEDSGYLSSGEEGEIRELVCALPALNVTERLNMEEAGLHNAVVAAEFVMVASEEAPVSKENMQTVDAIDDDAKNTALGRRPRIDELDDAAGASGSTAIGSGGEDEPVKKAKKKGSSQLHAPPQGPPQCNICGRNFTSWKAVFGHLRAHKDRGYQGFLPPPTFNATEEGFSAVVLASDSSRGGVLGFGSSGIDLNLDPMEEEEEAVSGFIPKFDLNRSPPQEGQEEEEEDKAK

SEQ ID NO: 45 - protein, 244 aa, unidentified
  source 1..244: note = Arachis ipaensis
MEKNHSTSASTTSLPSLSLSTPVTEPPSSPLVDQPNLSGEEEKLNILKTPEKILTDLTTNNDANGGSALPPSSPPPSPPQMMVSGAIATAVGGGAVVLGGNNIIGPIKRRKASEIMDPPSGTPTCPICSKTFSSWKGAFGHMRKHPDRQYRGFFKPPTFAPSSSKAATNASGNRHEAAGEEGSSAASGEVGEGGASPAVRNLLFDLNHPVEGVKDEEEGKIILGFDLNEPAVEEEEEEKEDKPF

SEQ ID NO: 46 - protein, 202 aa, Cajanus cajan
MQEQNPSENSPSNDDAPNPQGGEGEIMKTPWQVLEELCGGSSTPVAVAPVSEGVLQPKKRKTSEVRDPPRGKPICPVCNKEFTSWKAAFGHMRAHPNRDYRGFYKPPAFPSSSSSKDQPPPSANNNKGGGAKKTNAASDGTDEVDSGGGKEVTPSPNQLFGFDLNEPVEGLGSSHAVEEGVQEEKDLGFDLNEMPPAEDHEG

SEQ ID NO: 47 - protein, 265 aa, Brassica rapa
MTNPEKMKVDGDTEKEKEDSSDEQWSDEESAMREIVLGLPALSISSATFGVSIAVDEEEEARLNEQAVVAAELVIAAAEEAVMKEKSDGKKKKVRRQRKTMKLNNDDEAGGSSKGGEAAKKPRKKTSEFTNLPRGPPVCNVCGRAFHSWKAVFGHLRSHKDRSYRGFLPPPRFSAPIRGVMIPGPNSAFVRVVARGGSSGVVVASGGGGGGGASGGEGGRGVGIVMDVDRVEKEKQEVTESGTAAKFDLNKSPPKDDDEEEDKTK

SEQ ID NO: 48 - protein, 237 aa, Lagenaria siceraria
MEKNTNANANDNTVSETSPDQRHGGERSPMASSPPPSARNDDVNNPTPNVVIELSSSVVVAAPESTTTGDVRRRQTVDATLLDVGTSSHDHVGGSSDIEQGKKRGRGDGGEQQQQVKAAKKKGELTEVPKGEPRCATCNKVFKSWKALFGHLRSHPERTYRGALPPPTAAELDIRRCQQQLASTLLTVAQQVATSRRGLNIDLNQPSAADDGDSPEKTGSVGFDLNVEPPPESDDEK

SEQ ID NO: 49 - protein, 243 aa, unidentified
  source 1..243: note = Arachis ipaensis
MDKNASSSSSSSSSSSSSPSTSTTEQPPPPSPQDVPVEEPPVILKTPEEVLAELTESGESPPPPPPSAAEEGGIGGSAMAIAVGVGGEGVVLGEAGSGGSGAVGASRKRKASEVKDPPTGTPTCPVCNKTFASWKGAFGHMRKHPDRQYRGFFKPPTFTSSVAAAATSGATTQGFVEGGSSAASGEGGSVGAAGQVGEGGSFDLNQPVMERGGAGDGDEGRNGDEGKDLGFDLNKPADEEDKA

SEQ ID NO: 50 - protein, 271 aa, Prunus persica
MANQQNTTTNNTNRLASSAEGQQQPGNDNVNVNVTGVAATGTPSFPNPPPPSTVQLNVARGSSVSPPGLNRGPSGRGSSNPRRVSPSGRKSPPGGRGSPSSGRGSSSFASSPGAGQITQIGGRNFTPLSVEITIGGGSGTAGGSMIPGSSSGSPPRAKRKAEITAPEGVSPVCSVCKRSNFSSWKALFGHMRCHPERQWRGCFPPPGFEEAQRALQQGERLGALRGFDLNEASDPEEENEGGGGGGFDLNMLPPDEDKDGGGSSGGAAKTG

SEQ ID NO: 51 - protein, 321 aa, Glycine max
MQNQNPSEDDSTNKAPSDDTPHPTNNDNNNPPGESLIMKTVRQVLEELGGSRDDNDGGVGDGTPVAVALEVGGGGDGSGGDGGGVGGGPPVAMEASDGGDEDNDNEHSDSIGDSVPVAVAVKVGGRGGDNSGGGATVTVSVALSDGDYGGSNGSGGSPVAVAVAVGGGSTVMESVVVGTEGVQGRKRKTPLVRDPPTGRPTCPLCQKEFQTWKGAFGHMRAHPDRDYRGFFKPPVFGSPSSTQDQPPSDGKGDDSAKKSTGEDNTAEKGSASLPVRVPMFDLNELIEEDGSSHAAEPAEDMSTGEGKGSGFDLNEMPPAED

SEQ ID NO: 52 - protein, 355 aa, Glycine max
MNHHKYSSHRHNLFNSFLTLAIFIILGFSFSATGMQNQNPSEDDSTNKAPSDDTPHPTNNDNNNPPGESLIMKTVRQVLEELGGSRDDNDGGVGDGTPVAVALEVGGGGDGSGGDGGGVGGGPPVAMEASDGGDEDNDNEHSDSIGDSVPVAVAVKVGGRGGDNSGGGATVTVSVALSDGDYGGSNGSGGSPVAVAVAVGGGSTVMESVVVGTEGVQGRKRKTPLVRDPPTGRPTCPLCQKEFQTWKGAFGHMRAHPDRDYRGFFKPPVFGSPSSTQDQPPSDGKGDDSAKKSTGEDNTAEKGSASLPVRVPMFDLNELIEEDGSSHAAEPAEDMSTGEGKGSGFDLNEMPPAED

SEQ ID NO: 53 - protein, 357 aa, Glycine max
MNHHKYSSHRHNLFNSFLTLAIFIILGFSFSATGMQNQNPSEDDSTNKAPSDDTPHPTNNDNNNPPGESLIMKTVRQVLEELGGSRDDNDGGVGDGTPVAVALEVGGGGDGSGGDGGGVGGGPPVAMEASDGGDEDNDNEHSDSIGDSVPVAVAVKVGGRGGDNSGGGATVTVSVALSDGDYGGSNGSGGSPVAVAVAVGGGSTVMESVVVGTEGVQGRKRKTPLVRDPPTGRPTCPLCQKEFQTWKGAFGHMRAHPDRDYRGFFKPPVFGSPSSTQDQPPSDGKVAGDDSAKKSTGEDNTAEKGSASLPVRVPMFDLNELIEEDGSSHAAEPAEDMSTGEGKGSGFDLNEMPPAED

SEQ ID NO: 54 - protein, 324 aa, Glycine max
MQNQNPSEDDSTKKSPSSPSDDTSHPTNNNNNPQGESLVMKTVWQVLEELGGSGDDSDGDSGFGDGAPVAVAVGGGGDDNGDGDGSVGGGATVAMEKSDDDSDNNGDNSGSNLAVAVVVGGGDGSGSSGVGATVTVSVAVSGGGGNHGGGSSGGGGGGPVAVSVAVGGGSTVVESVVVSTEGVQGRKRKTSIVRDPPTGRPTCPLCQKEFPTWKGAFGHMRAHPDRDYRGFFKPPVFGSPSSTQDQPPRDDKVAGDDSAKKSSGEVNNGEKGSASSHVRVPMFDLNEPIEEEGSSHAAEPAEDMSSEEGKGFGFDLNEMPPAED

SEQ ID NO: 55 - protein, 280 aa, Cicer arietinum
MKRFCHSFHKTEKHDDYVWPLLAHEMKKNGSRIFICDLCDKSFSSGNALGGHKASHNRSDLLQPPIKKHKLTIDSCSLSSPNDHDDVKHKHACVLCHKVFPSNKALYGHMRSHSQKDSKAIQPPLITTTSRDSKIQSNNTDDQPILPAIDLEKYFPPRSHQTKKRCSKSTIDYELINVAQILCDMSRSDPTKFTTNIDNQRNKEHVTIVKNNNNPKKLVVTFKIPKDKTFKIPKDKEEKNKEAEMEKESSHQLGSRAVAKDFDLNEIPVDLDLVADEQAP

SEQ ID NO: 56 - protein, 290 aa, Cicer arietinum
MDVDELQAAEREDRRSIIIMKRSCQSFDKPMKNDDFVWPLLANEMKKNGSRIFICDLCGKSFSSGNALGGHKASHNRSNLLQPPIKKHKLTIDSCSLSSPHDHDDVKHKHACVLCHKVFPSNKVLYGHMRSHSQKDSKAIQPPPITTTSPDSKKQSNNTHDQPILPTIDLEKYFPPRSHQTKKRRSKSIIDYELINVAQILCDMSRSDPKRLKLSTNIDNQKNKEHVTIVKNNNNEKKLVVRFKIPKDKEVKNNEGETKKESSHQLGSRGVVKDFDLNEIPVDVADEQAN

SEQ ID NO: 57 - protein, 246 aa, Cicer arietinum
MKKNGSRIFICDLCGKSFSSGNALGGHKASHNRSNLLQPPIKKHKLTIDSCSLSSPHDHDDVKHKHACVLCHKVFPSNKVLYGHMRSHSQKDSKAIQPPPITTTSPDSKKQSNNTDDQPILPTIDLEKYFPPRSHQTKKRRSKSIIDYELINVAQILCDMSRSDPKRLKLSTNIDNQKNKEHVTIVKNNNNEKKLVVRFKIPKDKEVKNNEGETKKESSHQLGSRGVVKDFDLNEIPVDVADEQAN

SEQ ID NO: 58 - protein, 6 aa, synthetic construct
  REGION 1..6: note = EAR motif
  VARIANT 4..5: note = misc_feature - Xaa can be any naturally occurring amino acid
DLNXXP

SEQ ID NO: 59 - protein, 5 aa, synthetic construct
  REGION 1..5: note = EAR motif
  VARIANT 4: note = misc_feature - Xaa can be any naturally occurring amino acid
DLNXP

SEQ ID NO: 60 - DNA, 1335 bp, synthetic construct
  misc_feature 1..1335: note = Tar-MITE insert
  source 1..1335: mol_type = other DNA
actgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctac

SEQ ID NO: 61 - DNA, 2622 bp, synthetic construct
  misc_feature 1..2622: note = LsPromoter-with-deletion_LsCoding sequence_To3'UTR
  source 1..2622: mol_type = other DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaccgccacaaataaccgtcaccggaatacctataaatacaacttccatttcaatcgtctctctctgcactcccattctttgaatccaaaaaaggctacacacacacacagacaacaatggcagatgatggcaacaccgcccgtcaacaggctgacgccggtggccatggaaacgcacgtccaaactcatccacccccccttcgtcaccctcccaacctccaagaagaccacgccgtgccggagccactacaccgtcaaagctttcccaggcggcatcttcgtcaacaaatcttccaccgccgccaacgccaacaccaactccgcccaccccttctgccgacgggatattactaggtactgcaagaaggccggtaatatgcccgatttgcaaaaaggatatgtatcatgaaaaggcgctgtgtggccacatccggtggcacacgcaggaggagagactggcggcaagcagggatattgcccgggcgctttctgccaacgttgtttctgggcaaagaggcgatggagaacaagggccgtctaaacggttcaaactgccggatcttaacgagccaccaccgtcggaggactgattttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcatgatattgcaggaatgtaacttagaactcaacgataaaaagtgtgttgagatcgtggaagttgatcatttatcttgaattcataagataagcaacatgagataggataattaattactgtggggatttctttttatattcatattattcgttgtaatgcaaatttttaagtagaataacgaatgtgtcttattatttacggttaaatgcaaaagatcattaacatatctccattgatttgtttactgatgtattctcatgtgttctgttgctaaattctattttgaaaagtatattcaattaaagcaatgcattttagatataaagcaactccataacttataattgtgtagatttttaccaattcaatgtcttaaaagaaaaataaaaattgaaagtttaattaattgccatagtgatcccgttcccgtcaattatgtgatcaatttttaccagagcaaaaccccacatttcttgtagtgaacaatctgtggatgataaccgatcgaagacttttgtggggatttcactaaccaactatttggctctcattaattactattagttatataaggccatcccaagtattttggcaatattcaaaacgagtaaactataaaattacacttttggttcttgtatttctaacttttagaattatgtatttggttcttatagttttttatttgtatgaattacattacttttggtccacaatattttttattttctataattccatttttagtc

SEQ ID NO: 62 - protein, 300 aa, Arabidopsis thaliana
MRKMEAKKEEIKKGPWKAEEDEVLINHVKRYGPRDWSSIRSKGLLQRTGKSCRLRWVNKLRPNLKNGCKFSADEERTVIELQSEFGNKWARIATYLPGRTDNDVKNFWSSRQKRLARILHNSSDASSSSFNPKSSSSHRLKGKNVKPIRQSSQGFGLVEEEVTVSSSCSQMVPYSSDQVGDEVLRLPDLGVKLEHQPFAFGTDLVLAEYSDSQNDANQQAISPFSPESRELLARLDDPFYYDILGPADSSEPLFALPQPFFEPSPVPRRCRHVSKDEEADVFLDDFPADMFDQVDPIPSP

SEQ ID NO: 63 - DNA, 2338 bp, synthetic construct
  misc_feature 1..2338: note = LSpromoter - To coding sequence
  source 1..2338: mol_type = other DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaaaccagctctgtatatattggttatgttatgggttatatagagttgtttaaaaggcagttatcgattacgttctcagttctagagttttcacaaaaatcaattctcctgtatcacatcgaaagcaaaccggtctatttataaaagagatacatatttgaaacaacatattttttaattatatttatgtgttgtttataaatctagaaacccgtaaatttatatttagttcagtatccaatataggatctgaaactactttataaccgttaccagacaaataccggtcaagtttatattggtgtagttttcgattcctttgattttatatctttttggcgccatttcacgtcaacggtttaaaaacaagttatcgtttctccggtacagttaaccaccaaccgccacaaataaccgtcaccggaatacctataaatacaacttccatttcaatcgtctctctctgcactcccattctttgaatccaaaaaaggctacacacacacacagacaacaccaagtttgtacaaaaaagcaggctatggcagataatggcaacaccggccgtcaaaaggatgacgacggtggccatgatggaccacgccaaaacccaactactccaccctccccttcccgcacccctcgaagaccaaggcggaacacatcaccgcccaaacattctccgggggcgtcttcaagcaccatgccagcgccgcctactccccctgcgccgacgggaatcaccggtgcttctagttcttctgtgggtactaatataatttcatttattccacccaaaaccaaaagaacgaagtcggtgatctgcccgatctgcaacaaagatatgtgccatgagaaggcgctgtgtggccacatccggtggcatacacaggaggaaagattggcggccagcatcgctatagcaagagcgctatcttctaacgttgttgtttctggcaatggcgatgaagatgaaggtccatctaaaaagtataaactcccggacctgaacaaatctccaccgccggaggaggaggacgaggacgctgcctgattttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcacc

SEQ ID NO: 64 - DNA, 3673 bp, synthetic construct
  misc_feature 1..3673: note = LsPromoter-with-MITE_To Coding sequence
  source 1..3673: mol_type = other DNA
tcaatttcgatcttgactaccatgaatgtcatatttatatgacatacttataagaaattataatacaaatttgattttaaaataaaataaaaactattttcataacggatattttgtttcgtataaaaagaataccgtaagattaaattaaaataaacaatccgttaaactgacataatataatattggaaaataaaaaaatatatatgttttcataaaaaaaaaaacttcaagtttggatcagatttaggaaaagtaaaaccttatatttagttgatgaccgttttaccataaacaattatcagtttgtgctgggttaattgttaataaaaataatattaaaatttaattgaatataacatgaatatataatttttttttggaaaaacttcttttaaaatgttacataaattacgattaaatatttttaataccaaattatatagtatctggattaaagaggtcaaaaagtaaactttaaaaaataaaaaaaataaataatagattaatataagttagaggacttcaattatttttcatgtggacccttctaaaagaacctatttttacaaatagatttaaaaccaatctatttttggaaatggtctcctaaatcaatcaaattttataaaaaaactccaacactatcaaatatataacaattttattcgatcggttaaatagttagaattataacaaaataaaaaacaagataattttttcaaaataatttgaaataaaaaataaggtgtcaaaaatgaacttttatgtaaacttaacatcatttgtcatttttgttacaaaacatgattaatattattactttgtaagtgtccacttttcttcaaactccttgcttagttaaaaaaatctataatatatgaaattaattttagttttcttaaactacatatataattattttttatcaaacttgaattaaaagcctatgacaataaccaaaatgaaatagtatgacttatacatttgccgttgactaataaaccaaaaaatcgaattgacatcagaccaaaaccagctctgtatatattggttatgttatgggttatatagagttgtttaaaaggcagttatcgattacgttctcagttctagagttttcacaaaaatcaattctcctgtatcacatcgaaagcaaaccggtctatttataaaagagatacatatttgaaacaacatattttttaattatatttatgtgttgtttataaatctagaaacccgtaaatttatatttagttcagtatccaatataggatctgaaactactttataaccgttaccagacaaataccggtcaagtttatattggtgtagttttcgattcctttgattttatatctttttggcgccatttcacgtcaacggtttaaaaacaagttatcgtttctccggtacagttaaccaccactgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctacaaccgccacaaataaccgtcaccggaatacctataaatacaacttccatttcaatcgtctctctctgcactcccattctttgaatccaaaaaaggctacacacacacacagacaacaccaagtttgtacaaaaaagcaggctatggcagataatggcaacaccggccgtcaaaaggatgacgacggtggccatgatggaccacgccaaaacccaactactccaccctccccttcccgcacccctcgaagaccaaggcggaacacatcaccgcccaaacattctccgggggcgtcttcaagcaccatgccagcgccgcctactccccctgcgccgacgggaatcaccggtgcttctagttcttctgtgggtactaatataatttcatttattccacccaaaaccaaaagaacgaagtcggtgatctgcccgatctgcaacaaagatatgtgccatgagaaggcgctgtgtggccacatccggtggcatacacaggaggaaagattggcggccagcatcgctatagcaagagcgctatcttctaacgttgttgtttctggcaatggcgatgaagatgaaggtccatctaaaaagtataaactcccggacctgaacaaatctccaccgccggaggaggaggacgaggacgctgcctgattttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcacc

SEQ ID NO: 65 - DNA, 3405 bp, synthetic construct
  misc_feature 1..3405: note = ToPromoter_LsCoding sequence
  source 1..3405: mol_type = other DNA
taaggccataacaaggttatttgaacattcgcctaaaccctaaaccaactatatctattcggatttgttacgagttttagagtttctagaaaatcagttatatatcgattaggcttggttctaggttataagaaaaaatgattctctatccggtatgacatagagacaaaccggtctacttttataagaaaatcatatttgaaacaatgtctagttaataattaattaatatgtgcatgtttaaatttagaatatcctgaatttatatttagtgcgcaaccctatgtaagatcgaactaattttaaaccgtaaccagacatgcatgtttgatcagtaataaaaaggctattatcgtcgaaaatattaaaacaaaatataatatcttatttggcgccatttcatgtcaacggttgaaagcaagttaccaacgcctccaaccgatcatttctccggtaacaaccaactgctaccagggccggccagaaggctgtgcggcttgggctgtggcacaggggcagcagatttagtccatttattatggtctgctatttatttgggtccaataagatcagggcactaaaactcaaatacatcaattcaagtcggcggcaaaagaataaaaagcgccaatttttagaagacaaggagacagcacaataatcatgcttttccaatggccaattaattttagcctttgtgaggtataaatatgaatcactttttctccaaatatcattattgtaaactagtaaagagcgtaaattgttttatcgctaatcttcttctttttcaaatataattgtttgattctttgtagtaggtacgttaagttttatatcattcgtcctggaaaccttttcttatgccccttcaatttatgaatttgtgttttgtattgtcttgctattaatccaatgcaaactaattctgccgcatcgatctcacagagttggagcagcttaaaaggagttttggggcaacttaaaatgagggtagcttttttgttgttgttggtctactatttactttggtccttatcgttttagttttgtgttcattgaggatttgaatgactttgaacacaagtttataaaactaagtttttgtgattctaaatttttgatgtgtgcaaccgactaaatgaatataaaattctgatttgtgtatttctgatttctgatttaccggttattcattatgtttttctggatctcatatgaatttagtatataaaaatctcaggttcttcttactcagtctagtatattttggcattggggatcagggttaaaatcatcatttttctaagcactaacaagctttaaaaccatcaattaaaagttagttctccatacctggaattgtttattgctttagtattcatttcatagtgtcaataccttacaactgtttattgttttcaaatgtttactgttttgcactttagttgtttttagcatgtttcaactttcaaagttaattgacgatagtgtcatatacgaaatatcatcactcatacaaaattaaactggatagaattttttccgatgaaatctatttttaaatagttacgaatataataggagacttaatatgtttgtttcaaaattacacaaaaacaccatggaaatgggcattagtttgtataagcatccttttaaatataaagctttgttacattgatgccttctaaatatataatattgtaatgatttgtagtaaacaccatggaaatgggcattattaagatgtttcgcccagggcatgaaaaactatcggaccggccctgactgctactaataaccgccaccgcaaaaacctataaatacaacttccatcacaattctgtcccctactcccatttcaatccaaaaaaaggctacacagacatatacagcgccaagtttgtacaaaaaagcaggcttcatggcagatgatggcaacaccgcccgtcaacaggctgacgccggtggccatggaaacgcacgtccaaactcatccacccccccttcgtcaccctcccaacctccaagaagaccacgccgtgccggagccactacaccgtcaaagctttcccaggcggcatcttcgtcaacaaatcttccaccgccgccaacgccaacaccaactccgcccaccccttctgccgacgggatattactaggtactgcaagaaggccggtaatatgcccgatttgcaaaaaggatatgtatcatgaaaaggcgctgtgtggccatatacgttggcacacgcaagaggagagactggcggcaagcagggatattgcccgggcgctttctgccaacgttgtttctgggcaaagaggcgatggagaacaagggccgtctaaacggttcaaactgccggatcttaacgagccaccaccgtcggaggactgattttcttgcgggagaggcatgcatgtatgtgtggcctttttgcaataatactatgcatgggagatgcataaagtttatctttattttttttagtttaataatgtgtggcaaactttatatcatttatgatttcggccatgtactgctacatcgttctaggatggttattccctagaatttgctttatgtatttgttttctttgttaattcttctcttccttaaacgttatttcatgtgaattgcgatgtcatgatattgcaggaatgtaacttagaactcaacgataaaaagtgtgttgagatcgtggaagttgatcatttatcttgaattcataagataagcaacatgagataggataattaattactgtggggatttctttttatattcatattattcgttgtaatgcaaatttttaagtagaataacgaatgtgtcttattatttacggttaaatgcaaaagatcattaacatatctccattgatttgtttactgatgtattctcatgtgttctgttgctaaattctattttgaaaagtatattcaattaaagcaatgcattttagatataaagcaactccataacttataattgtgtagatttttaccaattcaatgtcttaaaagaaaaataaaaattgaaagtttaattaattgccatagtgatcccgttcccgtcaattatgtgatcaatttttaccagagcaaaaccccacatttcttgtagtgaacaatctgtggatgataaccgatcgaagacttttgtggggatttcactaaccaactatttggctctcattaattactattagttatataaggccatcccaagtattttggcaatattcaaaacgagtaaactataaaattacacttttggttcttgtatttctaacttttagaattatgtatttggttcttatagttttttatttgtatgaattacattacttttggtccacaatattttttattttctataattccatttttagtcgacc

SEQ ID NO: 66 - DNA, 354 bp, Taraxacum officinale
  source 1..354: mol_type = unassigned DNA
taattaatatgtgcatgtttaaatttagaatatcctgaatttatatttagtgcgcaaccctatgtaagatcgaactaattttaaaccgtaaccagacatgcatgtttgatcagtaataaaaaggctattatcgtcgaaaatattaaaacaaaatataatatcttatttggcgccatttcatgtcaacggttgaaagcaagttaccaacgcctccaaccgatcatttctccggtaacaaccaactgctactaataaccgccaccgcaaaaacctataaatacaacttccatcacaattctgtcccctactcccatttcaatccaaaaaaaggctacacagacatatacagcgatg

SEQ ID NO: 67 - DNA, 353 bp, Taraxacum officinale
  source 1..353: mol_type = unassigned DNA
taattaatatgtgtatgtttaaatttagaatatcctgaacttatatttagttcgcaaccctatgtaagatcgaactaattttaaaccgtaaccagacgtgcatgtttgatcagtaataaaaaggctatcatcgtcgaaaatattaaaacaaaatataatatcttatttggcgccatttcatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatg

SEQ ID NO: 68 - DNA, 353 bp, Taraxacum officinale
  source 1..353: mol_type = unassigned DNA
taattaatatgtgtatgtttaaatttagaatatcctgaacttatatttagttcgcaaccctatgtaagatcgaactaattttaaaccgtaaccagacatgcatgtttgatcagtaataaaaaggctatcatcgtcgaaaatattaaaacaaaatataatttcttatttggcgccatttcatgtcaacggttaaaagcaagttatcaacgcctccaaccgatcatttctccggtaaccaccaactgctactaataaccgccaccgcaaaaacctatatatacaacttccatcacaattctgtcccctactcccatttcaatccaaaaaaggctacacagacatatacagcgatg

SEQ ID NO: 69 - DNA, 354 bp, Taraxacum sp.
  source 1..354: mol_type = unassigned DNA
taaataatatgtgtatgtttaaatttagaatatcctgaatttatatttagtgcgcaaccctatgtaagatcgaactaattttaaaccgtaaccagacatgcatgtttgatcagtaataaaaaggctattatcgtcgaaaatattaaaacaaaatataatatcttattcggcgccatttcatgtcaacggttaaaagcaagttaccaacgcctccaaccgatcatttctccggtaaccaccaactgctactaataaccgccaccgcaaaaacctataaatacaacttccatcacaattctgtcccctactcccatttcaatccaaaaaaaggctacacagacatatacagcgatg

(19) United States
(12) Patent Application Publication
(10) Pub. No.: US 2026/0234653 A1
(43) Pub. Date: Aug. 13, 2026

(54) MODIFIED PROMOTER OF A PARTHENOGENESIS GENE

(71) Applicant: Keygene N.V., Wageningen (NL)

(72) Inventors: Rik Hubertus Martinus OP DEN CAMP, Wageningen (NL); Peter Johannes VAN DIJK, Wageningen (NL)

(73) Assignee: Keygene N.V., Wageningen (NL)

(21) Appl. No.: 19/354,684

(22) Filed: Oct. 9, 2025

Related U.S. Application Data
(63) Continuation of application No. 18/133,392, filed on Apr. 11, 2023, now Pat. No. 12,460,224.
Application No. 18/133,392 is a continuation of application No. PCT/EP21/78281, filed on Oct. 13, 2021.

(30) Foreign Application Priority Data
Oct. 13, 2020 (EP) 20201578.0

(51) Int. Cl. C12N 15/82; A01H 1/02

(52) U.S. Cl. CPC C12N 15/8287; A01H 1/022

(57) ABSTRACT

The invention provides a method to produce a mutant gene, wherein said gen comprises a modified promoter and wherein said gene is capable of inducing the parthenogenesis phenotype to a plant. The invention further provides said mutant gene, isolated nucleic acid molecule, construct or vector comprising the same. Also, the invention provides for a method to produce a parthenogenetic plant comprising the mutant gene, and the parthenogenetic plant thus obtained.

### CROSS-REFERENCE TO RELATED APPLICATIONS

This application is a continuation of U.S. application Ser. No. 18/133,392 filed Apr. 11, 2023, which is a continuation of International Patent Application No. PCT/EP2021/078281 filed Oct. 13, 2021, which application claims priority to European Patent Application No. 20201578.0 filed Oct. 13, 2020, the contents of which are all incorporated herein by reference in their entireties.

### SEQUENCE LISTING

The instant application contains a Sequence Listing which has been submitted electronically in XML format and is hereby incorporated by reference in its entirety. Said XML copy, created on Sep. 4, 2024, is named 085342-4500_SL.xml and is 185,381 bytes in size.

### FIELD OF THE INVENTION

The present invention relates to the field of biotechnology and in particular to plant biotechnology including plant breeding. The invention relates in particular to the identification and uses of genes relating to and useful e.g. in apomixis and haploid induction. The invention in particular relates to a mutant promoter of the gene that is associated with parthenogenesis. The invention further relates to methods for inducing parthenogenesis in plants and crops, to the use of the gene and/or the promoter for apomixis in particular in combination with apomeiotic gene(s), or for the production of haploid plants of which the chromosomes can be doubled to produce doubled haploids.

### BACKGROUND OF THE INVENTION

Apomixis (also called agamospermy) is asexual plant reproduction through seeds. Apomixis has been reported in some 400 flowering plant species (Bicknell and Koltunow, 2004). Apomixis in flowering plants occurs in two forms:
(1) gametophytic apomixis, in which the embryo arises from an unreduced, unfertilized egg cell by parthenogenesis;
(2) sporophytic apomixis in which the embryo arises somatically from a sporophytic cell.

Examples of gametophytic apomicts are dandelions (Taraxacum sp.), hawkweeds (Hieracium sp.), Kentucky blue grass (Poa pratensis) and eastern gamagrass (Tripsacum dactyloides). Examples of sporophytic apomixis are citrus (Citrus sp.) and mangosteen (Garcinia mangostana). Gametophytic apomixis involves two developmental processes:
(1) the avoidance of meiotic recombination and reduction (apomeiosis); and
(2) development of the egg cell into an embryo, without fertilization (parthenogenesis).

Apomictically produced seeds are genetically identical to the parental plant. It has been recognized since long that apomixis can be extremely useful in plant breeding (Asker, 1979; Hermsen, 1980; Asker and Jerling, 1990; Vielle-Calzada et al., 1995). The most obvious advantage of the introduction of apomixis into crops is the true breeding of heterotic F1 hybrids. In most crops F1 hybrids are the best performing varieties. However, in sexual crops F1 hybrids have to be produced each generation again by crossing of inbred homozygous parents, because self-fertilization of F1 hybrids causes loss of heterosis by recombination in the genomes of the F2 progeny plants. Producing sexual F1 seeds is a recurrent, complicated and costly process. In contrast, apomictic F1 hybrids would breed true eternally. In other words, genetic fixation of F1 hybrids and production of uniform progeny plants through seed becomes possible.

F1 fixation by apomixis is a special case of the general property of apomixis that any genotype, whatever its genetic complexity, would breed true in one step. This implies that apomixis could be used for immediate fixation of polygenic quantitative traits. It should be noted that most yield traits are polygenic. Apomixis could be used for the stacking (or pyramiding) of multiple traits (for example various resistances, several transgenes, or multiple quantitative trait loci). Without apomixis, in order to fix such a suite of traits, each trait locus must be made homozygous individually and later on combined. As the number of loci involved in a trait increases, the making of these trait loci homozygous by crossing becomes time consuming, logistically challenging and thereby costly. Moreover specific epistatic interactions between alleles are lost by homozygosity. With apomixis it becomes possible to fix this type of non-additive genetic variation. Therefore, apomixis, clonal reproduction through seeds, has the potential to cause of paradigm shift in plant breeding, commercial seed production and agriculture (van Dijk et al. 2016).

Besides instantaneously fixing any genotype, whatever its complexity, there are important additional agricultural uses of apomixis. Sexual interspecific hybrids and autopolyploids often suffer from sterility due to meiotic problems. Since apomixis skips meiosis, with apomixis these problems of interspecific hybrids and autopolyploids can be solved. Since apomixis prevents female hybridization, apomixis coupled with male sterility has been proposed for the containment of transgenes, preventing transgene introgression in wild relatives of transgenic crops (Daniell, 2002). In insect pollinated crops (e.g. Brassica) apomictic seed set would not be limited by insufficient pollinator services. This is becoming more important in the light of the increasing health problems of pollinating bee populations (Varroa mite infections, African killer bees etc.). In tuber propagated crops, like potato, apomixis would maintain the superior genotype clonally, but reduce or even remove the current risk of virus transmission and related cost in clean production, containment and certification. Also the storage costs of apomictic seeds are much less than that of tubers or other vegetatively propagated plant parts. In ornamentals apomixis could replace labour intensive and expensive tissue culture propagation. It is thought that in general apomixis strongly reduces the costs of cultivar development and plant propagation.

Unfortunately apomixis does not occur in any of the major crops. There have been numerous attempts to introduce apomixis in sexual crops. For instance, introgression of apomixis genes, mutation of sexual model species, de novo generation of apomixis by hybridization, and cloning of candidate genes. Introgression of apomixis genes from wild apomicts into crop species through wide crosses have not been successful so far (e.g. apomixis from Tripsacum dactyloides into maize—Savidan, Y., 2001; Morgan et al., 1998; WO97/10704). As to mutating sexual model species, WO2007/066214 describes the use of an apomeiosis mutant called Dyadin Arabidopsis. However, the Dyadis a recessive mutation with very low penetrance. In a crop species this mutation is of limited use. Generation of apomixis de novo by hybridization between two sexual ecotypes has not resulted in agronomical interesting apomicts (US2004/0168216 A1 and US2005/0155111 A1). Cloning of candidate apomixis genes by transposon tagging in maize has been described in US2004/0148667. Orthologs of the elongate gene have been claimed, which are supposed to induce apomixis. However, according to Barrell and Grossniklaus (2005), the elongate gene skips meiosis II and therefore does not maintain the maternal genotype, which makes it much less useful.

It has been described in US2006/0179498 that so called Reverse Breeding would be an alternative for apomixis. However, this is a technically complicated in vitro laboratory procedure, whereas apomixis is an in vivo procedure that is carried out by the plants themselves. Moreover, with reverse breeding, once the parental lines have been reconstructed (doubled gamete homozygotes) crossing still has to be carried out.

Apomixis in natural apomicts generally has a genetic basis (reviewed by Ozias-Akins and Van Dijk, 2007). Therefore an alternative method could be the isolation of apomixis genes from natural apomictic species. However this is not an easy task, because natural apomicts often have a polyploid genome and positional cloning in polyploids is very difficult. Other complicating factors are suppression of recombination in apomixis specific chromosomal regions, repetitive sequences and segregation distortion in crosses.

As described herein, there is a need for procedures for inducing apomixis in crops, which are devoid of at least some of the limitations of the present state of the art. Particularly, there is a need for methods for producing apomictic plants and apomictic seeds. There is also a need to provide for genes and proteins involved in the developmental processes of apomixis, particularly parthenogenesis, which are suitable for use in introducing apomixis or (doubled) haploids in crops and which can substantially mimic apomictic pathways.

### SUMMARY OF THE INVENTION

The inventors have identified and isolated the parthenogenesis locus and gene, the alleles associated with the parthenogenetic phenotype (indicated herein as the parthenogenetic allele or Par allele) and the non-parthenogenetic phenotype (indicated herein as the sexual allele of the parthenogenesis gene or par allele), their genetic sequences, i.e. promoter sequences, 5′UTR, coding sequences, 3′UTR sequences and encoded protein sequences.

The present invention provides for a method for modifying or altering a parallele into a Parallele, by altering the promoter sequence of said par allele. The present invention is in particular useful for modifying an endogenous par allele into a Par allele, preferably by random or targeted mutagenesis, and optionally by transformation. The resulting mutant allele is capable of transformation a plant and/or its offspring into a plant having, or having an increased, capability of developing an egg cell into an embryo without fertilization.

### Definitions

As used herein, the term “locus” (plural: loci) means a specific place (or places) or a site on a chromosome where for example a gene or genetic marker is found. For example, the “parthenogenesis locus” refers to the position in the genome where the parthenogenesis gene is located. Two functional variants have been identified for the parthenogenesis gene, i.e. an allele contributing to the parthenogenetic phenotype, indicated herein as the parthenogenetic allele of the parthenogenesis gene or Par allele, and/or its sexual counterpart(s), indicated herein as a sexual allele of the parthenogenesis gene or par allele.

A gene, allele, protein or nucleic acid being “functional in parthenogenesis” is to be understood herein as contributing to the parthenogenetic phenotype and/or increasing or converting the ability to a plant or plant cell to develop an egg cell into an embryo.

A “Parthenogenesis gene” is a gene that is associated with parthenogenesis, wherein “associated with” is to be understood herein as indicative for the parthenogenetic or the non-parthenogenetic (sexual) phenotype. The genetic sequences of the (dominant) allele (the Par allele) conferring parthenogenesis and its two sexual counterparts (the par alleles or sexual alleles of the parthenogenesis gene) have been identified for the first time in the triploid apomict Taraxacum officinale isolate A68, as described in PCT/EP2020/064991, which is incorporated herein by reference. Preferably, the Par allele initiates parthenogenesis. The (dominant) allele conferring parthenogenesis has the genetic sequence of SEQ ID NO: 5 and comprises a promoter having the sequence of SEQ ID NO: 2, a coding sequence having the sequence of SEQ ID NO: 3 and a 3′UTR having the sequence of SEQ ID NO: 4. One of the sexual alleles has the genetic sequence of SEQ ID NO: 10 and comprises a promoter having the sequence of SEQ ID NO: 7, a coding sequence having the sequence of SEQ ID NO: 8 and a 3′UTR having the sequence of SEQ ID NO: 9. The other sexual allele has the genetic sequence of SEQ ID NO: 15 and comprises promoter having the sequence of SEQ ID NO: 12, a coding sequence having the sequence of SEQ ID NO: 13 and a 3′UTR having the sequence of SEQ ID NO: 14. Orthologous genes have been identified in other species based on the characteristic features of the proteins (indicated as the PAR proteins) encoded by these parthenogenesis genes. The presence of a PAR protein in an egg cell may lead to the repression of inhibitors of embryogenesis, triggering cell division without fertilization. These PAR proteins are characterized in that they comprise a zinc finger C2H2-type domain (IPR13087), preferably a zinc finger K2-2-like domain having the consensus sequence C.{2}C.{7}[K/R]A.{2}GH.[R/N].H, which can also be annotated as: CXXCXXXXXXX[K/R]AXXGHX[R/N]XH (SEQ ID NO: 37), wherein X may be any naturally occurring amino acid, wherein [K/R] indicates that the amino acid is lysine or arginine, and wherein R/N] indicates that the amino acid is arginine or asparagine (see Englbrecht et al., 2004). In addition to the zinc finger C2H2-type domain, preferably a zinc finger K2-2-like domain as defined herein, the protein comprises an EAR (Ethylene-responsive element binding factor-associated Amphiphilic Repression) motif having the consensus amino acid sequence DLNXXP (SEQ ID NO: 58) or DLNXP (SEQ ID NO: 59), wherein X may by any naturally occurring amino acid (see Kagale et al., 2010; and Yang et al., 2018). Preferably said EAR motif is located C-terminal. Preferably the zinc finger C2H2-type domain is located N-terminal of said EAR motif. Preferably, the PAR protein has a length of at most 400 amino acids, wherein said protein comprises one or two EAR motifs as indicated herein and a zinc finger K2-2-like domain as defined herein. Preferably, the protein has a length of at most 400 amino acids, wherein said protein comprises only one or two EAR motifs as indicated herein and only one zinc finger K2-2-like domain as defined herein, i.e. no further EAR motifs as defined herein and no further zinc finger K2-2-like domains as defined herein. In addition to the features of the maximum size of 400 amino acids, the only one or two EAR motifs as indicated herein and a single zinc finger K2-2-like domain as defined, the PAR protein may comprise only one further zinc finger domain having the zinc finger consensus sequence of C.{2}C.{12}H.{3}H, which can also be annotated as: CXXCXXXXXXXXXXXXHXXXH (SEQ ID NO: 38), but more preferably comprises no further zinc finger domains having the zinc finger consensus sequence of C.{2}C.{12}H.{3}H (SEQ ID NO: 38). Preferably, the PAR protein has a length of at most 500 amino acids. The PAR protein may be between about 50-500, 100-300 or between about 150-200 amino acids. The PAR protein may have a length of about 170 amino acids. Orthologous parthenogenesis genes may be, but are not limited to, genes encoding for any one of the PAR proteins selected from the group consisting of: PAR protein from Ananas comosus (e.g. UniProtKB: AOA199URK4), PAR protein from Apostasia shenzhenica (e.g. UniProtKB: A0A2I0AZW3), PAR protein from Arabidopsis thaliana (e.g. UniProtKB: Q8GXP9, A0A178V2S4, 081793, A0A178V1 Q3, A0MFC1, 081801), PAR protein from Arabidopsis lyrata subsp. Lyrata (e.g. UniProtKB: D7MC52 or D7MCE8), PAR protein from Arachis ipaensis (e.g. SEQ ID NO: 45 or SEQ ID NO: 49), PAR protein from Brachypodium distachyon (e.g. UniProtKB: 11J0D9), PAR protein from Brassica oleracea var. oleracea (e.g. UniProtKB: A0A0D3A1Q6 or A0A0D3A1 Q3), PAR protein from Brassica campestris (e.g. UniProtKB: A0A398AHT1), PAR protein from Brassica rapa (e.g. SEQ ID NO: 47), PAR protein from Brassica rapa subsp. Pekinensis (e.g. UniProtKB: M4D574 or M4D571), PAR protein from Brassica oleracea (e.g. UniProtKB: A0A3P6ESB1 or A0A3P6F726), PAR protein from Brassica campestris (e.g. UniProtKB: A0A3P5ZMM3 or A0A3P5Z1M1), PAR protein from Cajanus cajan (e.g.SEQ ID NO: 46), PAR protein from Capsella rubella (e.g. UniProtKB: ROH2J1 or ROHOC2), PAR protein from Cephalotus follicularis (e.g. UniProtKB: A0A1Q3CSK1), PAR protein from Cicer arietinum (e.g. UniProtKB: A0A3Q7YBZ1, A0A1S2YZL9, A0A3Q7Y0Z6 or A0A1S2YZM6; or SEQ ID NO: 55, 56 or 57), PAR protein in Cichorium endivia (e.g. SEQ ID NO: 39), PAR protein from Cucumis sativus (e.g. UniProtKB: A0A0A0KGW4 or A0A0A0L0X7), PAR protein from Cucumis melo (e.g. UniProtKB: A0A1S3BLF2 or A0A1S3B298), PAR protein from Cucumis sativus (e.g. UniProtKB: A0A0A0KAW8), PAR protein from Cucurbita moschata (e.g. SEQ ID NO: 43), PAR protein from Cuscuta campestris (e.g. UniProtKB: A0A484MGR1), PAR protein from Dendrobium catenatum (e.g. UniProtKB: A0A2I0V7N9, A0A2I0X2T2 or A0A2I0W0Q8), PAR protein from Dorcoceras hygrometricum (e.g. UniProtKB: A0A2Z7D3Y1), PAR protein from Eutrema salsugineum (e.g. UniProtKB: V4LSH0; or SEQ ID NO: 44), PAR protein from Fagus sylvatica (e.g. UniProtKB: A0A2N9E5Y5, A0A2N9HAB9, or A0A2N9H993), PAR protein from Genlisea aurea (e.g. UniProtKB: S8E1M6), PAR protein from Glycine max (e.g. SEQ ID NO: 51, 52, 53 or 54), PAR protein from Gossypium hirsutum (e.g. UniProtKB: A0A1U8LDU9), PAR protein from Helianthus annuus (e.g. SEQ ID NO: 21), PAR protein from Hevea brasiliensis (e.g. SEQ ID NO: 42), PAR protein in Hieracium aurantiacum (e.g. SEQ ID NO: 40), PAR protein from Juglans regia (e.g. UniProtKB: A0A214E6B1), PAR protein from Lactuca sativa (e.g. UniProtKB: A0A2J6KZF7; or SEQ ID NO: 22), PAR protein from Lagenaria siceraria (e.g. SEQ ID NO: 48), PAR protein from Medicago truncatula (e.g. UniProtKB: G7K024), PAR protein from Morus notabilis (e.g. UniProtKB: W9SMY3 or W9SMQ7), PAR protein from Mucuna pruriens (e.g. UniProtKB: A0A371 ELJ8), PAR protein from Nicotiana attenuata (e.g. UniProtKB: A0A1J61QI6), PAR protein from Nicotiana sylvestris (e.g. UniProtKB: A0A1U7VXJ0), PAR protein from Nicotiana tabacum (e.g. UniProtKB: A0A1 S4A651 or A0A1 S3YHQ2), PAR protein from Oryza sativa subsp. Japonica (e.g. UniProtKB: B9FGH8), PAR protein from Oryza barthii (e.g. UniProtKB: A0A0D3FWX3), PAR protein from Panicum miliaceum (e.g. UniProtKB: A0A3L6Q010 or A0A3L6T1 D6), PAR protein from Parasponia andersonii(e.g. UniProtKB: A0A2P5BMI5), PAR protein from Populus alba (e.g. UniProtKB: A0A4U5PSY9), PAR protein from Populus trichocarpa (e.g. UniProtKB: B9H661), PAR protein from Punica granatum (e.g. UniProtKB: A0A2101BB9, A0A218XB85 or A0A218W102), PAR protein from Senecio cambrensis (e.g. SEQ ID NO: 41), PAR protein from Prunus persica (e.g. SEQ ID NO: 50), PAR protein from Trema orientale (e.g. UniProtKB: A0A2P5EB04), PAR protein from Trifolium pratense (e.g. UniProtKB: A0A2K3N851), PAR protein from Trifolium subterraneum (e.g. UniProtKB: A0A2Z6MYD3 or A0A2Z6MDR7), PAR protein from Trifolium pratense (e.g. UniProtKB: A0A2K3PR44), PAR protein from Vitis vinifera (e.g. UniProtKB: A0A438C778, A0A438ESC4 or A0A438DBR4) and PAR protein from Zea mays (e.g. UniProtKB: A0A1 D6HF46, B6UAC5, A0A3L6F4S1, A0A3L6EMC6, A0A3L6EMC6, K7UHQ6 or A0A1 D6KHZ4). Such gene may also encode for a PAR protein selected from the group consisting of: PAR protein from Actinidia chinensis (e.g. UniProtKB: A0A2R6S2S9), PAR protein from Beta vulgaris (e.g. UniProtKB: XP_010690656.1), PAR protein from Solanum tuberosum (e.g. UniProtKB: XP_015159151.1), PAR protein from Solanum lycopersicum (e.g. UniProtKB: A0A3Q7GXB3, Solyc05g055500 or Solyc06g060480), PAR protein from Capsicum baccatum (e.g. UniProtKB: A0A2G2WJR7), PAR protein from Solanum melongena (e.g. UniProtKB: AVC18974.1), PAR protein from Glycine soja (e.g. GeneBank accession: XP_028201014.1, XP_006596577.1 or UniprotKB: A0A445M3M6), PAR protein from Arachis hypogaea (e.g. UniProtKB: A0A444WUX5), PAR protein from Phaseolus vulgaris (e.g. UniProtKB: V7CIF6), PAR protein from Daucus carota (e.g. GeneBank accession: XP_017245413.1), PAR protein from Triticum aestivum (e.g. UniProtKB: A0A3B6RP64), PAR protein from Oryza sativa subsp. indica (e.g. UniProtKB: A2YH63), PAR protein from Oryza sativa subsp. japonica (e.g. UniProtKB: Q5Z7P5) and PAR protein from Theobroma cacao (e.g. UniProtKB: A0A061 DL63). Optionally, an orthologous gene is a gene encoding for a PAR protein that comprises or consists of an amino acid sequence that has at least about 50%, 60%, 70%, 80%, 90%, 95%, 98%, 99% or more identity to any one of SEQ ID NO: 1, 6 and 11, respectively, and/or any one of the orthologues provided herein above preferably when aligned pairwise using e.g. the Needleman and Wunsch algorithm (global sequence alignment) with default parameters and as compared over their whole length.

As used herein, the term “allele(s)” means any of one or more alternative forms of a gene at a particular locus. In a diploid and/or polyploid cell of an organism, alleles of a given gene are located at a specific location, or locus on a chromosome, wherein one allele is present on each chromosome of the set of homologous chromosomes. A diploid and/or polyploid organism or plant species may comprise a large number of different alleles at a particular locus.

The term “dominant allele” as used herein refers the relationship between alleles of one gene in which the effect of one allele (i.e. the dominant allele) masks the contribution of a second allele (i.e. the recessive allele) at the same locus. For genes on an autosome (any chromosome other than a sex chromosome), the alleles and their associated traits are autosomal dominant or autosomal recessive. Dominance is a key concept in Mendelian inheritance and classical genetics. Optionally, a dominant allele codes for a functional protein whereas the recessive allele does not. Optionally, a dominant allele and a recessive allele may encode the same, or substantially the same, functional protein, while only the dominant allele, unlike a recessive allele, is capable of expressing a certain amount of said functional protein under a specific circumstance and/or in a particular tissue thereby converting a particular phenotype like parthenogenesis.

The term “female ovary” (plural: “ovaries”) as used herein refers to an enclosure in which spores are formed. It can be composed of a single cell or can be multicellular. All plants, fungi, and many other lineages form ovaries at some point in their life cycle. Ovaries can produce spores by mitosis or meiosis. Generally, within each ovary, meiosis of a megaspore mother cell produces four haploid megaspores. In gymnosperms and angiosperms, only one of these four megaspores is functional at maturity, and the other three degenerate. The megaspore that pertains divides mitotically and develops into the female gametophyte (megagametophyte), which eventually produces one egg cell.

The term “female gamete” as used herein refers to a cell that fuses under normal (sexual) circumstances with another (“male”) cell during fertilization (conception) in organisms that sexually reproduce. In species that produce two morphologically distinct types of gametes, and in which each individual produces only one type, a female is any individual that produces the larger type of gamete (called an ovule (ovum) or egg cell). In plants, the female ovule is produced by the ovary of the flower. When mature, the haploid ovule produces the female gamete which is then ready for fertilization. The male cell is (mostly haploid) pollen and is produced by the anther.

The term “genetic marker” or “polymorphic marker” refers to a region on the genomic DNA which can be used to “mark” a particular location on the chromosome. If a genetic marker is tightly linked to a gene or is ‘on’ a gene it “marks” the DNA on which the gene is found and can therefore be used in a (molecular) marker assay to select for or against the presence of the gene, e.g. in marker assisted breeding/selection (MAS) methods. Examples of genetic markers are AFLP (amplified fragment length polymorphism, EP534858), microsatellite, RFLP (restriction fragment length polymorphism), STS (sequence tagged site), SNP (Single Nucleotide Polymorphism), SFP (Single Feature Polymorphism; see Borevitz et al., 2003), SCAR (sequence characterized amplified region), CAPS markers (cleaved amplified polymorphic sequence) and the like. The further away the marker is from the gene, the more likely it is that recombination (crossing over) takes place between the marker and the gene, whereby the linkage (and co-segregation of marker and gene) is lost. The distance between genetic loci is measured in terms of recombination frequencies and is given in cM (centiMorgans; 1 cM is a meiotic recombination frequency between two markers of 1%). As genome sizes vary greatly between species, the actual physical distance represented by 1 cM (i.e. the kilobases, kb, between two markers) also varies greatly between species.

It is understood that, when referring to “linked” markers herein, this also encompasses markers “on” the gene itself.

“MAS” refers to “marker assisted selection”, whereby plants are screened for the presence and/or absence of one or more genetic and/or phenotypic markers in order to accelerate the transfer of the DNA region comprising the marker (and optionally lacking flanking regions) into an (elite) breeding line.

A “molecular marker assay” (or test) refers to a (DNA based) assay that indicates (directly or indirectly) the presence or absence of an allele e.g. a Par or par allele in a plant or plant part. Preferably it allows one to determine whether a particular allele is homozygous or heterozygous at the parthenogenesis locus in any individual plant. For example, in one embodiment a nucleic acid linked to the parthenogenesis locus is amplified using PCR primers, the amplification product is digested enzymatically and, based on the electrophoretically resolved patterns of the amplification product, one can determine which allele(s) is/are present in any individual plant and the zygosity of the allele at the parthenogenesis locus (i.e. the genotype at each locus). Examples are SCAR markers (sequence characterized amplified region), CAPS markers (cleaved amplified polymorphic sequence) and similar marker assays.

As used herein, the term “heterozygous” means a genetic condition existing when two different alleles reside at a specific locus, but are positioned individually on corresponding sets of homologous chromosomes in the cell. Conversely, as used herein, the term “homozygous” means a genetic condition existing when two (or more in case of polyploidy) identical alleles reside at a specific locus, but are positioned individually on corresponding sets of homologous chromosomes in the cell.

A “variety” is used herein in conformity with the UPOV convention and refers to a plant grouping within a single botanical taxon of the lowest known rank, which grouping can be defined by the expression of the characteristics and can be distinguished from any other plant grouping by the expression of at least one of the said characteristics and is considered as a unit with regard to its suitability for being propagated unchanged (stable).

The terms “protein” or “polypeptide” are used interchangeably and refer to molecules consisting of a chain of amino acids, without reference to a specific mode of action, size, 3 dimensional structure or origin. A “fragment” or “portion” of a protein may thus still be referred to as a “protein”.

The term “gene” means a DNA sequence comprising a region (transcribed region), which is transcribed into an RNA molecule (e.g. an pre-mRNA which is processed to an mRNA) in a cell, operably linked to suitable regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites.

A “chimeric gene” (or recombinant gene) refers to any gene, which is not normally found in nature in a species, in particular a gene in which one or more parts of the nucleotide sequence are present that are not associated with each other in nature. For example the promoter is not associated in nature with part or all of the transcribed region or with another regulatory region.

A “native gene” refers to any gene comprising a promoter sequence, a coding sequence, and optionally a 3′-OTR sequence that can also be found in a naturally occurring gene. Optionally, the nucleotide sequence of a native gene is identical to a sequence found in nature. It is understood herein that the native gene can be a transgene, In this embodiment, the native gene is present in a plant species, wherein the plant species does not naturally comprise said native gene.

An “endogenous gene” is understood herein as a native gene in its natural environment, i.e. present in a plant species in which it is naturally comprised.

A “3′UTR” or “3′ non-translated sequence” (also often referred to as 3′ untranslated region, or 3′end) refers to the nucleotide sequence found downstream of the coding sequence of a gene, which comprises for example a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

A “5′UTR” or “leader sequence” or “5′ untranslated region” is a region of the mRNA transcript, and the corresponding DNA, between the +1 position where mRNA transcription begins and the translation start codon of the coding region (usually AUG on the mRNA or ATG on the DNA). The 5′UTR usually contains sites important for translation, mRNA stability and/or turnover, and other regulatory elements.

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). An active protein may refer to a protein being capable of fulfilling its function, which may for instance be inhibition of expression of a certain gene by binding to regulatory elements of a 5′UTR of said gene. An active protein in certain embodiments refers to a protein being constitutively active. The coding sequence is preferably in sense-orientation and encodes a desired, biologically active protein or peptide, or an active peptide fragment. In gene silencing approaches, the DNA sequence is preferably present in the form of an antisense DNA or an inverted repeat DNA, comprising a short sequence of the target gene in antisense or in sense and antisense orientation.

A “transcription regulatory sequence” is herein defined as a nucleotide sequence that is capable of regulating the rate of transcription of a (coding) sequence operably linked to the transcription regulatory sequence. A transcription regulatory sequence as herein defined will thus comprise all of the sequence elements necessary for initiation of transcription (promoter elements), for maintaining and for regulating transcription, including e.g. attenuators or enhancers. Although mostly the upstream (5′) transcription regulatory sequences of a coding sequence are referred to, regulatory sequences found downstream (3′) of a coding sequence are also encompassed by this definition.

As used herein, the term “promoter” refers to a nucleic acid fragment that functions to control the transcription of one or more DNA regions, located upstream with respect to the direction of transcription of the transcription initiation site, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. Optionally the term “promoter” includes herein also the 5′UTR region (e.g. the promoter may herein include one or more parts upstream (5′) of the translation initiation codon of a gene, as this region may have a role in regulating transcription and/or translation.

A “constitutive” promoter is a promoter that is active in most tissues under most physiological and developmental conditions. An “inducible” promoter is a promoter that is physiologically (e.g. by external application of certain compounds) or developmentally regulated. A “tissue specific” promoter is only active in specific types of tissues or cells. A “promoter active in plants or plant cells” refers to the general capability of the promoter to drive transcription within a plant or plant cell. It does not make any implications about the spatiotemporal activity of the promoter.

As used herein, the term “operably linked” refers to a linkage of polynucleotide elements in a functional relationship. A nucleic acid is “operably linked” when it is placed into a functional relationship with another nucleotide sequence. For instance, a promoter, or rather a transcription regulatory sequence, is operably linked to a coding sequence if it affects the transcription of the coding sequence. Operably linked means that the DNA sequences being linked are typically contiguous and, where necessary to join a promoter sequence to a protein coding sequence or a protein coding sequence to a 3′UTR. A “nucleic acid construct” or “vector” is herein understood to mean a man-made nucleic acid molecule resulting from the use of recombinant DNA technology and which is used to deliver exogenous DNA into a host cell. The vector backbone may for example be a binary or superbinary vector (see e.g. U.S. Pat. No. 5,591,616, US 2002138879 and WO95/06722), a co-integrate vector or a T-DNA vector, as known in the art and as described elsewhere herein, into which a gene or chimeric gene is integrated or, if a suitable transcription regulatory sequence is already present, only a desired nucleotide sequence (e.g. a coding sequence, an antisense or an inverted repeat sequence) is integrated downstream of the transcription regulatory sequence. Vectors usually comprise further genetic elements to facilitate their use in molecular cloning, such as e.g. selectable markers, multiple cloning sites and the like.

A “recombinant host cell” or “transformed cell” or “transgenic cell” are terms referring to a new individual cell (or organism) arising as a result of at least one nucleic acid molecule, especially comprising a transgene and/or chimeric gene encoding a desired protein or a nucleotide sequence which upon expression yields a specific protein such as the PAR protein as defined herein, having been introduced into said cell. An “isolated nucleic acid” is used to refer to a nucleic acid which is no longer in its natural environment, for example in vitro or in a recombinant bacterial or plant host cell.

A “host cell” is the original cell to be transformed with a transgene to become a recombinant host cell. The host cell is preferably a plant cell or a bacterial cell. The recombinant host cell may contain the nucleic acid construct as an extra-chromosomally (episomal) replicating molecule, or more preferably, comprises the gene or chimeric gene integrated in the nuclear or plastid genome of the host cell.

A “recombinant plant” or “recombinant plant part” or “transgenic plant” is a plant or plant part (seed or fruit or leaves, for example) which comprises a recombinant gene or chimeric gene or transgene, even though the gene may not be expressed, or not be expressed in all cells.

An “elite event” is a recombinant plant which has been selected to comprise the recombinant gene or transgene at a position in the genome which results in good phenotypic and/or agronomic characteristics of the plant. The flanking DNA of the integration site can be sequenced to characterize the integration site and distinguish the event from other transgenic plants comprising the same recombinant gene at other locations in the genome.

The term “selectable marker” is a term familiar to one of ordinary skill in the art and is used herein to describe any genetic entity which, when expressed, can be used to select for a cell or cells containing the selectable marker. Selectable marker gene products confer for example antibiotic resistance, or more preferably, herbicide resistance or another selectable trait such as a phenotypic trait (e.g. a change in pigmentation) or a nutritional requirement. The term “reporter” is mainly used to refer to visible markers, such as green fluorescent protein (GFP), eGFP, luciferase, GUS and the like.

The term “orthologue” of a gene or protein refers herein to the homologous gene or protein found in another species, which has the same function as the gene or protein, but (usually) diverged in sequence from the time point on when the species harboring the genes diverged (i.e. the genes evolved from a common ancestor by speciation). Orthologues of the Taxaracum parthenogenesis gene may thus be identified in other plant species based on both sequence comparisons (e.g. based on percentages sequence identity over the entire sequence or over specific domains) and functional analysis.

The terms “homologous” and “heterologous” refer to the relationship between a nucleic acid or amino acid sequence and its host cell or organism, especially in the context of transgenic organisms. A homologous sequence is thus naturally found in the host species (e.g. a lettuce plant transformed with a lettuce gene), while a heterologous sequence is not naturally found in the host cell (e.g. a lettuce plant transformed with a sequence from potato plants). Depending on the context, the term “homologue” or “homologous” may alternatively refer to sequences which are descendent from a common ancestral sequence (e.g. they may be orthologues).

“Stringent hybridization conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridizes to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridizations (Northern blots using a probe of e.g. 100nt) are for example those which include at least one wash in 0.2×SSC at 63° C. for 20 min, or equivalent conditions. Stringent conditions for DNA-DNA hybridization (Southern blots using a probe of e.g. 100nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“High stringency” conditions can be provided, for example, by hybridization at 65° C. in an aqueous solution containing 6×SSC (20×SSC contains 3.0 M NaCl, 0.3 M Na-citrate, pH 7.0), 5×Denhardt's (100×Denhardt's contains 2% Ficoll, 2% Polyvinyl pyrollidone, 2% Bovine Serum Albumin), 0.5% sodium dodecyl sulphate (SDS), and 20 μg/ml denaturated carrier DNA (single-stranded fish sperm DNA, with an average length of 120-3000 nucleotides) as non-specific competitor. Following hybridization, high stringency washing may be done in several steps, with a final wash (about 30 min) at the hybridization temperature in 0.2-0.1×SSC, 0.1% SDS.

“Moderate stringency” refers to conditions equivalent to hybridization in the above described solution but at about 60-62° C. In that case the final wash is performed at the hybridization temperature in 1×SSC, 0.1% SDS.

“Low stringency” refers to conditions equivalent to hybridization in the above described solution at about 50-52° C. In that case, the final wash is performed at the hybridization temperature in 2×SSC, 0.1% SDS. See also Sambrook et al. (1989) and Sambrook and Russell (2001).

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman Wunsch) which aligns the sequences optimally over the entire length, while sequences of substantially different lengths are preferably aligned using a local alignment algorithm (e.g. Smith Waterman). Sequences may then be referred to as “substantially identical” or “essentially similar” when they (when optimally aligned by for example the programs GAP or BESTFIT using default parameters) share at least a certain minimal percentage of sequence identity (as defined herein). The percent of sequence identity is preferably determined using the “BESTFIT” or “GAP” program of the Sequence Analysis Software Package™ (Version 10; Genetics Computer Group, Inc., Madison, Wis.). GAP uses the Needleman and Wunsch global alignment algorithm (Needleman and Wunsch, Journal of Molecular Biology 48:443-453, 1970) to align two sequences over their entire length (full length), maximizing the number of matches and minimizing the number of gaps. A global alignment is suitably used to determine sequence identity when the two sequences have similar lengths. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blossum62 for proteins and DNAFull for DNA). “BESTFIT” performs an optimal alignment of the best segment of similarity between two sequences and inserts gaps to maximize the number of matches using the local homology algorithm of Smith and Waterman (Smith and Waterman, Advances in Applied Mathematics, 2:482-489, 1981, Smith et al., Nucleic Acids Research 11:2205-2220, 1983). When sequences have a substantially different overall lengths, local alignments, such as those using the Smith Waterman algorithm, are preferred.

As used herein “sequence identity” refers to the extent to which two optimally aligned polynucleotide or peptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in reference sequence segment, i.e., the entire reference sequence or a smaller defined part of the reference sequence. “Percent identity” is the identity fraction times 100.

Useful methods for determining sequence identity are also disclosed in Guide to Huge Computers, Martin J. Bishop, ed., Academic Press, San Diego, 1994, and Carillo, H., and Lipton, D., Applied Math (1988) 48:1073. More particularly, preferred computer programs for determining sequence identity include the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; Altschul et al., J. Mol. Biol. 215:403-410 (1990); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity.

Alternatively percentage similarity or identity may be determined by searching against public databases, using algorithms such as FASTA, BLAST, etc. Thus, the nucleic acid and protein sequences of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., BLASTx and BLASTn) can be used. See the homepage of the National Center for Biotechnology Information at http://www.ncbi.nlm.nih.gov/.

The term “sexual plant reproduction” as used herein refers to a developmental pathway where a (e.g. diploid) somatic cell referred to as the “megaspore mother cell” undergoes meiosis to produce four reduced megaspores. One of these megaspores divides mitotically to form the megagametophyte (also known as the embryo sac), which contains a reduced egg cell (i.e. cell having a reduced number of chromosomes compared to the mother) and two reduced polar nuclei. Fertilization of the egg cell by one sperm cell of the pollen grain generates a (e.g. diploid) embryo, while fertilization of the two polar nuclei by the second sperm cell generates the (e.g. triploid) endosperm (process referred to as double fertilization).

The term “megaspore mother cell” or “megasporocyte” as used herein refers to a cell that produces megaspores by reduction, usually meiosis, to create four haploid megaspores which will develop into female gametophytes. In angiosperms (also known as flowering plants), the megaspore mother cell produces a megaspore that develops into a megagametophyte through two distinct processes including megasporogenesis (formation of the megaspore in the nucellus, or megasporangium), and megagametogenesis (development of the megaspore into the megagametophyte).

The term “asexual plant reproduction” as used herein is a process by which plant reproduction is achieved without fertilization and without the fusion of gametes. Asexual reproduction produces new individuals, genetically identical to the parent plants and to each other, except when mutations or somatic recombinations occur. Plants have two main types of asexual reproduction including vegetative reproduction (i.e. involves budding, tillering, etc of a vegetative piece of the original plant) and apomixis.

The term “apomixis” as used herein refers to the formation of seeds by asexual processes. One form of apomixis is characterized by: 1) apomeiosis, which refers to the formation of unreduced embryo sacs in the ovary, and 2) parthenogenesis, which refers to the development of the unreduced egg into an embryo. A few hundred wild plant species feature apomictic reproduction and propagate asexually. Apomeiosis is a process that results into the production of unreduced egg cells, with the same chromosome number and identical or highly similar genotype as the somatic tissue of the mother plant. The unreduced egg cells can be derived from an unreduced megaspore (diplospory) or from a somatic initial cell (apospory). In the case of diplospory, megasporogenesis is replaced by a mitotic division or by a modified meiosis. The modified meiosis is preferably of the first division restitution type, without recombination. Alternatively the modified meiosis can be of the second division restitution type. In a preferred embodiment, apomeiosis is of the diplosporous type affecting the first meiotic division. Apomixis is known to occur in different forms including at least two forms known as gametophytic apomixis and sporophytic apomixis (also referred to as adventive embryony). Examples of plants where gametophytic apomixis occurs include dandelion (Taraxacum sp.), hawkweed (Hieracium sp.), Kentucky blue grass (Poa pratensis), eastern gamagrass (Tripsacum dactyloides) and others. Examples of plants where sporophytic apomixis occurs include citrus (Citrus sp.) mangosteen (Garcinia mangostana) and others.

The term “diplospory” as used herein refers to a situation where an unreduced embryo sac is derived from the megaspore mother cell either directly by mitotic division or by aborted meiotic events. Three major types of diplospory have been reported, named after the plants in which they occur, and they are the Taraxacum, Ixeris and Antennaria types. In the Taraxacum type, the meiotic prophase is initiated but then the process is aborted resulting in two unreduced dyads one of which gives rise to the embryo sac by mitotic division. In the Ixeris type, two further mitotic divisions of the nuclei to give rise to an eight-nucleate embryo sac follow equational division following meiotic prophase. The Taraxacum and Ixeris types are known as meiotic diplospory because they involve modifications of meiosis. By contrast, in the Antennaria type, referred to as mitotic diplospory, the megaspore mother cell does not initiate meiosis and directly divides three times to produce the unreduced embryo sac. In gametophytic apomixis by diplospory, an unreduced gametophyte is produced from an unreduced megaspore. This unreduced megaspore results from either a mitotic-like division (mitotic displory) or a modified meiosis (meiotic displory). In both gametophytic apomixis by apospory and gametophytic apomixis by diplospory, the unreduced egg cell develops parthenogenetically into an embryo. Apomixis in Taraxacum is of the diplosporous type, which means that the first female reduction division (meiosis I) is skipped, resulting in two unreduced megaspores with the same genotypes as the mother plant. One of these megaspores degenerates and the other surviving unreduced megaspore gives rise to the unreduced megagametophyte (or embryo sac), containing an unreduced egg cell. This unreduced egg cell develops without fertilization into an embryo with the same genotype as the mother plant. The seeds resulting from the process of gametophytic apomixis are referred to as apomictic seeds.

The term “diplospory function” refers to the capability to induce diplospory in a plant, preferably in the female ovary, preferably in a megaspore mother cell and/or in a female gamete. Thus a plant in which diplospory function is introduced, is capable of performing the diplospory process, i.e. producing unreduced gametes via a meiosis I restitution.

The term “apomictic seeds” as used herein refers to seeds, which are obtained from apomictic plant species or by plants or crops induced to undergo apomixis, particularly gametophytic apomixis through diplospory. Apomictic seeds are characterised in that they are a clone and genetically identical to the parent plant and germinate plants that are capable of true breeding. In the present invention, the “apomictic seeds” also refers to “clonal apomictic seeds”.

The term “apomictic plant(s)” as used herein, refers to a plant that reproduce itself asexually, without fertilization. An apomictic plant may be a sexual plant that has been modified to become apomictic, e.g. a sexual plant, which has for instance been genetically modified with one or more of the parthenogenesis genes as taught herein so as to obtain an apomictic plant, or a plant that is the progeny of an apomictic plant. In that case, apomictically produced offspring are genetically identical to the parent plant.

A “clone” of a cell, plant, plant part or seed is characterized in that they are genetically identical to their siblings as well as to the parent plant from which they are derived. Genomic DNA sequences of individual clones are nearly identical, however, mutations may cause minor differences.

The term “true breeding” or “true breeding organism” (also known as pure-bred organism) as used herein refers to an organism that always passes down a certain phenotypic trait unchanged or nearly unchanged to its offspring. An organism is referred to as true breeding for each trait to which this applies, and the term “true breeding” is also used to describe individual genetic traits.

The term “F1 hybrid” (or filial 1 hybrid) as used herein refers to the first filial generation of offspring of distinctly different parental types. The parental types may or may not be inbred lines. F1 hybrids are used in genetics, and in selective breeding, where it may appear as F1 crossbreed. The offspring of distinctly different parental types produce a new, uniform phenotype with a combination of characteristics from the parents. F1 hybrids are associated with distinct advantages such as heterosis, and thus are highly desired in agricultural practice. In an embodiment of the invention, the methods, genes, proteins, variants or fragments thereof as taught herein can be used to fix the genotype of F1 hybrids, regardless of its genetic complexity, and allows production of organisms that can breed true in one step.

The term “pollination” or “pollinating” as used herein refers to the process by which pollen is transferred from the anther (male part) to the stigma (female part) of the plant, thereby enabling fertilization and reproduction. It is unique to the angiosperms, the flower-bearing plants. Each pollen grain is a male haploid gametophyte, adapted to being transported to the female gametophyte, where it can effect fertilization by producing the male gamete (or gametes), in the process of double fertilization. A successful angiosperm pollen grain (gametophyte) containing the male gametes is transported to the stigma, where it germinates and its pollen tube grows down the style to the ovary. Its two gametes travel down the tube to where the gametophyte(s) containing the female gametes are held within the carpel. One nucleus fuses with the polar bodies to produce the endosperm tissues, and the other with the ovule to produce the embryo.

The term “parthenogenesis” as used herein refers to a form of asexual reproduction in which growth and development of embryos occur without fertilization. The genes and proteins of the invention can, preferably in combination with a diplosporous factor, for instance a gene or chemical factor, produce apomictic offspring.

The term “parthenogenesis phenotype” as used herein refers to the ability of a plant and/or its offspring to grow and develop an embryo from an egg cell without fertilization.

The term “pyramiding or stacking gene” as used herein, refers to the process of combining related or unrelated genes from different parental line into one plant, which underlie desirable or favourable traits (e.g. disease resistance traits, colour, drought resistance, pest resistance, etc.). Pyramiding or stacking gene can be performed using traditional breeding methods or can be accelerated by using molecular markers to identify and keep plants that contain the desired allele combination and discard those that do not have the desired allele combination. In an embodiment of the present invention, the parthenogenesis genes as taught herein may be advantageously used in gene pyramiding or stacking program to produce apomictic plants or to introduce apomixis in sexual crops.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. amino acids) are referred to.

As used herein, the term “plant” includes plant cells, plant tissues or organs, plant protoplasts, plant cell tissue cultures from which plants can be regenerated, plant calli, plant cell clumps, and plant cells that are intact in plants, or parts of plants, such as embryos, pollen, ovules, fruit, flowers, leaves (e.g. harvested lettuce crops), seeds, roots, root tips and the like.

### DETAILED DESCRIPTION OF THE INVENTION

The present invention provides a method for producing a mutant gene, wherein the mutant gene is functional in parthenogenesis. Preferably, the method comprises a step of mutating the promoter sequence of a sexual variant of the parthenogenesis gene indicated herein as the par allele to increase expression of the encoded PAR protein. The resulting mutant gene may be considered a Par allele as it is capable of inducing the parthenogenesis phenotype to a plant. In case the parallele is an endogenous allele of a plant or plant cell lacking a dominant Par allele, the method of the invention results in the transformation of a plant or plant cell not showing parthenogenesis into a plant or plant cell showing parthenogenesis by modification of the promoter of said par allele. The present invention provides a method to convert the sexual parallele into a parthenogenetic Parallele, by modification of the promoter sequence of the par allele. The present invention provides a method for producing a mutant gene functional in parthenogenesis, comprising the steps of:
(a) providing a gene comprising a sequence encoding a PAR protein operably linked to a promoter; and
(b) modifying the promoter by modifying the sequence upstream of the sequence encoding the PAR protein to increase expression of the encoded PAR protein, preferably in the mature female gametophyte.

More in particular, the present invention provides a method for producing a mutant gene functional in parthenogenesis, comprising the steps of:
(a) providing a gene comprising a sequence encoding a PAR protein operably linked to a promoter comprising one or more transcription factor MYB binding sites; and
(b) modifying the promoter by modifying the sequence upstream of the one or more transcription factor MYB binding sites to increase expression of the encoded PAR protein, preferably in the mature female gametophyte.

The sequence upstream of the sequence encoding a PAR protein, preferably upstream one or more transcription factor MYB binding sites, may be modified by introducing an enhancer sequence that enhances expression of the PAR protein and/or by removing a repressor sequence that represses expression of the PAR protein. Preferably, an enhancer sequence is inserted, preferably a female gametophyte-specific enhancer sequence. Said insert may be a MITE sequence as defined herein.

Alternatively or in addition, the promoter sequence upstream of the sequence encoding a PAR protein, preferably upstream one or more MYB binding sites, is altered by (random) mutagenesis resulting in one or more nucleotide substitutions, insertions and/or deletions, to introduce one or more enhancer sequences and/or to increase expression of the encoded PAR protein.

In addition or alternatively, the present invention provides a method for producing a mutant gene functional in parthenogenesis, comprising the steps of:
(a) providing a gene comprising a sequence encoding a PAR protein operably linked to a promoter comprising one or more transcription factor MYB binding sites; and
(b) modifying the promoter by modifying at least one of the one or more transcription factor MYB binding sites to increase expression of the encoded PAR protein.

Preferably, the modified promoter of the mutant gene of the invention results, when present in a plant, in increased expression of the PAR protein encoded by the coding sequence operably linked to said promoter, as compared to the unmodified counterpart, i.e. the (endogenous or native) promoter from which the promoter of the invention is obtained as defined herein. Preferably, said increased expression is at least in the egg cell of the plant comprising the modified promoter and/or mutant gene of the invention. Preferably, the increase in expression is an increase of at least 10, 20, 30, 40, 50, 60, 70, 80, 90 or 100% as compared to the original non-mutant gene (i.e. the gene of step (a) of the method of the invention).

In an embodiment the gene encoding a PAR protein in step a) is not a gene originating from Arabidopsis thaliana. In an embodiment, the mutant gene of the invention does not comprise a mutant promoter derived from Arabidopsis thaliana DAZ3 having a mutation in one or more of the MYB binding sites.

The gene provided in step (a) is a parthenogenesis gene, preferably a sexual variant of the gene, i.e. a parallele. As the resulting mutant gene is functional in parthenogenesis, the method of the invention may also be considered as a method for converting a sexual allele of a parthenogenesis gene (parallele) into an allele that is functional in parthenogenesis (Par allele).

A transcription factor MYB binding site (also indicated herein as “MYB binding site”) is a sequence within a promoter recognized and bound by the transcription factor MYB. MYB proteins are a family of DNA binding proteins comprising varying numbers of MYB domain repeats conferring their ability to bind DNA at a MYB binding site and thereby regulating transcription.

The inventors identified one or more MYB binding sites in a region of about 50-150 bps upstream of the start codon of the par allele. A MYB binding site is defined herein as a sequence of preferably 7 nucleotides that preferably has the nucleotide sequence of NACCNNN, preferably AACCNNN, more preferably AACCGNN, even more preferably AACCG[C/T]N, even more preferably AACCG[C/T]C, and may be AACCGCC, AACCGTC or [T/A]AACCGCC (Borg et al., 2011). Preferably one or more MYB binding site is located between about 60-140 bps, 70-130 bps, 80-120 bps or 90-110 bps upstream of the start codon of the sequence encoding the PAR protein, preferably at most about 200, 190, 180, 170, 160 or 150, 140, 130, 120 or 110 nucleotides upstream of the start codon, even more preferably the 3′ terminus of the one or more, preferably two, MYB binding site is located 85, 86, 87, 88, 89, 90, 91, 92, 93, 94, 95, 96, 97, 98, 99, 100, 101, 102, 103, 104, 105, 106, 107, 108, 109 or 110 nucleotides upstream of the start codon of the sequence encoding the PAR protein. In some instances, two MYB binding sites are located in said region. For instance, in lettuce (Lactuca sativa) a first MYB binding site is located at position 117-110 nucleotides upstream of the ATG start codon and a second MYB binding site is located at the position 104-98 nucleotides upstream of the of the ATG start codon.

The MYB binding site preferably acts as a binding site of the transcription factor MYB, wherein the MYB transcription factor is an R2R3 transcription factor or an R2R3-MYB transcription factor, wherein said transcription factor MYB may be, or may be variant, homologue or orthologue of, DUO1 (UniProtKB accession A0A178VEK7). The transcription factor preferably has at least 80%, 85%, 90%, 95%, 96%, 97%, 98%, 99% or 100% sequence identity with SEQ ID NO: 62.

In addition or alternatively, the invention may also be considered as a method for producing a mutant gene capable of inducing the parthenogenetic phenotype to a plant, wherein said method comprises the steps of:
(a) providing a gene comprising a sequence encoding a PAR protein, wherein the sequence is operably linked to a promoter, wherein the promoter optionally comprises one or more transcription factor MYB binding sites; and
(b) modifying the promoter to increase expression of the encoded PAR protein, preferably by at least one of:
i) introducing an insert in the promoter sequence upstream of the start codon of the sequence encoding a PAR protein as defined herein, even more preferably upstream or directly upstream of the one or more MYB binding sites as defined herein, to increase expression of the encoded PAR protein preferably by introducing an enhancer sequence or removing a repressor sequence;
ii) introducing a substitution and/or deletion in the promoter sequence upstream of the start codon of the sequence encoding a PAR protein as defined herein, even more preferably upstream or directly upstream of the one or more MYB binding sites as defined herein, wherein the substitution and/or deletion increases expression of the encoded PAR protein preferably by introducing an enhancer sequence or removing a repressor sequence; and
(iii) a combination of (i) and (ii).

Modifying the promoter in the method of the invention may be performed using any conventional method known in the art, such as, but not limited to, introducing an insert or a deletion in the promoter directly upstream of the one or more MYB binding sites as defined herein preferably by random or targeted mutagenesis, optionally through homologous recombination.

Optionally, the insert, substitution or deletion in the promoter may modify or remove one or more MYB binding sites as defined herein.

Hence the invention may also be considered a method for producing a mutant gene capable of inducing the parthenogenetic phenotype to a plant, wherein said method comprises the steps of:
(a) providing a gene comprising a sequence encoding a PAR protein, wherein the sequence is operably linked to a promoter comprising one or more transcription factor MYB binding sites; and
(b) modifying the promoter by modifying the sequence upstream of the one or more transcription factor MYB binding sites to increase expression of the encoded PAR protein by introducing an insert or a deletion in the promoter directly upstream of the one or more MYB binding sites in the promoter as defined herein.

In addition, or alternative, the method of the invention may comprise modifying the promoter by inducing, modifying or removing one or more MYB binding sites as defined herein.

In case the promoter comprises one or more, preferably two, MYB binding sites as defined herein, optionally these one or more, preferably two, MYB binding sites are modified or removed to reduce binding of the transcription factor MYB and/or an insert or deletion is introduced upstream of these one or more MYB binding sites. Modification of a MYB binding site in the method of the invention may be a modification of 1, 2, 3, 4, 5, 6, or 7 nucleotides, preferably of 1, 2 or 3 nucleotides, even more preferably of 1 nucleotide, such that the sequence is no longer a MYB binding site. Preferably said modification is at least a mutation (nucleotide exchange, insertion or deletion) located at the first, second, third, fourth or fifth position of the 7 nucleotides long MYB binding motif as indicated above, i.e. a modification of at least one of the A, A, C, C and G of the motif AACCGNN. Preferably, said modification is at least a mutation (nucleotide exchange, insertion or deletion) located at the first, second, third or fourth position of the 7 nucleotides long MYB binding motif as indicated above, i.e. a modification of at least one of the A, A, C and C of the motive AACCGNN, optionally, a modification of two, three or all four the nucleotides on these positions.

Preferably, the modification results in a decreased or abolished binding of the transcription factor MYB to the MYB binding site. Preferably, the binding affinity is decreased at least about 10%, 20%, 30%, 40%, 50%, 60%, 70%, 80%, 90%, 95%, or 100% when tested under suitable experimental conditions, e.g. as described in Kelemen et al., supra. Optionally, the modification of a MYB binding site may be the deletion of said MYB binding site. Said modification may be performed by random mutagenesis (for instance by chemical or radiation mutagenesis) or targeted mutagenesis (for instance CRISPR-endonuclease mediated mutagenesis). Optionally, multiple (two, three or more) MYB binding sites present in the gene are modified as defined herein in the method of the invention. Optionally, the mutant gene comprises a modified promoter of Lactuca sativa, wherein said promoter comprises or consists of the sequence of SEQ ID NO: 17. Optionally, the mutant gene comprises said promoter operably linked to the sequence encoding a Lactuca sativa PAR protein, preferably said coding sequence comprises or consists of the sequence of SEQ ID NO: 33. Optionally, the mutant gene comprises or consists of the sequence of SEQ ID NO: 35. The invention also comprises a plant or plant cell, preferably a Lactuca sativa plant or plant cell, comprising the mutant gene and/or construct.

In an embodiment, the promoter of a sexual gene, preferably the pargene, may be modified by introducing an insert upstream of the one or more MYB binding sites. The insert may be introduced in close vicinity or directly next to the one or more MYB binding sites. The distance between the insert and the MYB binding site, or in case of multiple MYB binding site, the MYB binding site located most upstream from the coding sequence, is preferably at most 10, 9, 8, 7, 6, 5, 4, 3, 2, 1 or 0 nucleotides. Preferably, the insert is introduced directly upstream of the one or more MYB binding sites. The insert that may be introduced in the promoter of the gene in the method of the invention, preferably comprises a nucleic acid insert, preferably a double-stranded DNA insert, wherein said insert has a length of between 50 and 2000 bp, between 100 and 1900 bp, between 200 and 1800 bp, between 300 and 1700 bp, between 400 and 1600 bp, between 500 and 1500 bp, between 600 and 1400 bp, between 1000 and 1400, between 1200 and 1400, or between 1300 and 1400 bp. Even more preferably, said insert has a length of about 1300 bp. Alternatively or in addition, the insert is between about 1-50 bp, between about 5-30 bp or in between about 10-20 bp. Preferably, said insert is introduced within a promoter upstream (5′) of the MYB binding site as defined herein, preferably such that the distance between the MYB binding site and the insert, preferably the 3′-end of the insert, is between 0-200 bps, preferably at most 0, 10, 20, 50, 60, 70, 80, 90, 100, 110, 120, 130, 140, 150, 160, 170, 180, 190 or 200 bps. Preferably, said insert is localized such that the 3′ end nucleotide of the insert is at a position that is homologous to the position of nucleotide 1798 of SEQ ID NO: 2 and/or of nucleotide 1798 of SEQ ID NO: 5. Preferably, said insert is devoid of an open reading frame.

Said insert may be a non-autonomous transposable element, preferably a hAT-derived non-autonomous transposon element. The insert may comprise an enhancer element, preferably a female gametophyte-specific enhancer element. Even more preferably said insert is a Miniature Inverted-Repeat Transposable Elements (MITE) or MITE-like sequence, wherein said MITE or MITE-like sequence is a non-autonomous element, preferably a non-autonomous transposable element, characterized that contains an internal sequence devoid of an open reading frame, that is flanked by terminal inverted repeats (TIRs) which in turn are flanked by small direct repeats (target site duplications, TSDs). The TIRs may have the sequence CAGGGCCGG and/or CCGGCCCTG. The TSD may have the sequence ACTGCTAC. For a further description of MITE, TIR and sequences, referred is to Guo et al, Scientific Reports. 2017 Jun. 1; 7(1):2634 which is incorporated herein by reference. Said insert, preferably said MITE or MITE-like sequence, may have at least about 50%, 60%, 70%, 80%, 90%, 95%, 98%, 99% or more identity to SEQ ID NO: 60. The insert may be introduced synthetically, by recombination and/or targeted genome editing.

Optionally, the mutant gene comprises a modified promoter of Lactuca sativa, wherein said promoter comprises or consists of the sequence of SEQ ID NO: 18. Optionally, the mutant gene comprises said promoter operably linked to the sequence encoding a Lactuca sativa PAR protein, preferably said coding sequence comprises or consists of the sequence of SEQ ID NO: 33. Optionally, the mutant gene comprises or consists of the sequence of SEQ ID NO: 36. Optionally, the mutant gene comprises a modified promoter of Lactuca sativa, wherein said promoter comprises or consists of the sequence of SEQ ID NO: 18 and said promoter is operably linked to the sequence encoding a Taraxacum officinale PAR protein, preferably said coding sequence comprises or consists of the sequence of SEQ ID NO: 3. Optionally, the mutant gene comprises or consists of the sequence of SEQ ID NO: 64. The invention also comprises a plant or plant cell, preferably a Lactuca sativa plant or plant cell, comprising the mutant gene and/or construct.

Optionally the mutant gene comprises a Par promoter of Taraxacum officinale, wherein said promoter comprises or consists of the sequence of SEQ ID NO: 2. Optionally, the mutant gene comprises said promoter operably linked to the sequence encoding a PAR protein, wherein said PAR protein is not the (native) Taraxacum officinale PAR protein. Optionally, the mutant gene comprises said promoter operably linked to the sequence encoding an orthologous PAR protein as defined herein above.

Optionally, the mutant gene comprises a Par promoter of Taraxacum officinale, wherein said promoter comprises or consists of the sequence of SEQ ID NO: 2, and wherein the promoter is operably linked to the sequence encoding a Lactuca sativa PAR protein, preferably said coding sequence comprises or consists of the sequence of SEQ ID NO: 33. Optionally, the mutant gene comprises or consists of the sequence of SEQ ID NO: 65.

In another embodiment, the promoter of a sexual gene, preferably the pargene, may be modified by introducing a deletion upstream of the one or more MYB binding sites. The deletion may be introduced in close vicinity or directly next to the one or more MYB binding sites. The distance between the deletion and the MYB binding site, or in case of multiple MYB binding site, the MYB binding site located most upstream from the coding sequence, is preferably at most 200, 150, 50, 40, 30, 20, 10, 9, 8, 7, 6, 5, 4, 3, 2 or 1 nucleotide. Preferably, the deletion is introduced directly upstream of the one or more MYB binding sites. The deletion that may be introduced in the promoter of the gene in the method of the invention, preferably comprises a nucleic acid deletion, preferably a double-stranded DNA deletion, wherein said deletion has a length of between 10 and 1000 bp, between 50 and 900 bp, between 100 and 800 bp, between 200 and 700 bp, between 350 and 600 bp, preferably about 400 bp. Preferably, said deletion is devoid of an open reading frame. The deletion may be introduced by recombination and/or targeted or random genome editing. Optionally, the mutant gene comprises a modified promoter of Lactuca sativa, wherein said promoter comprises or consists of the sequence of SEQ ID NO: 20. Optionally, the mutant gene comprises said promoter operably linked to the sequence encoding a Lactuca sativa PAR protein, preferably said coding sequence comprises or consists of the sequence of SEQ ID NO: 33. Optionally, the mutant gene comprises or consists of the sequence of SEQ ID NO: 61. The invention also comprises a plant or plant cell, preferably a Lactuca sativa plant or plant cell, comprising the mutant gene and/or construct.

Preferably, the gene of step a) of the method of the invention, and the mutant gene as obtainable by the method of the invention is, or is part of, a nucleic acid molecule, preferably a DNA molecule, even more preferably a genomic DNA molecule. Optionally, said genomic DNA molecule is in a plant cell, preferably a plant protoplast. Modification of the promoter of the sexual gene by the method of the invention and when located in a plant cell, may result in a significant increase in parthenogenesis phenotype of the plant derived from said plant cell as compared to a control plant, wherein preferably the control plant does not show the parthenogenesis phenotype while the plant comprising the mutant gene of the invention shows the parthenogenesis phenotype. The control plant preferably only differs from the plant derived from said plant cell in that the promoter of the par allele is not modified as defined herein. Preferably, the control plant or control plant cell only differs from respectively a plant cell or plant of the invention in that the control plant or control plant cell does not comprise a genetic modification as defined herein.

Preferably, the gene of step (a) is a par allele and modification of the promoter of the par allele as defined herein and when located in a plant cell that is not capable of parthenogenesis, results in a plant that shows the parthenogenesis phenotype. As the mutant gene obtainable by the method of the invention is functional in parthenogenesis, the mutant gene may be considered a Par allele, and the method of the invention may also be considered a method to convert a parallele into a Parallele.

Optionally, modification of the promoter by the method of the invention affects binding of the transcription factor MYB to the promoter, resulting in increased expression of the PAR protein encoded by the gene comprising the promoter. Optionally, the modification results in decreased or abolished binding of the transcription factor MYB. Binding of the transcription factor MYB to the promoter can be evaluated by any assay suitable known by the person skilled in the art, such as, but not limited to in vivo yeast one-hybrid systems (e.g. see Kelemen et al., PLoS One. 2015; 10(10): e0141044).

Random mutagenesis may be, but is not limited to, chemical mutagenesis, gamma radiation, X-ray or fast neutron radiation. Non-limiting examples of chemical mutagenesis include, but are not limited to, EMS (ethyl methanesulfonate), MMS (methyl methanesulfonate), NaN3 (sodium azide) D), ENU (N-ethyl-N-nitrosourea), AzaC (azacytidine) and NQO (4-nitroquinoline 1-oxide). Optionally, mutagenesis systems such as TILLING (Targeting Induced Local Lesions IN Genomics; McCallum et al., 2000, Nat Biotech 18:455, and McCallum et al. 2000, Plant Physiol. 123, 439-442, both incorporated herein by reference) may be used to generate plant lines with a modified gene as defined herein. TILLING uses traditional chemical mutagenesis (e.g. EMS mutagenesis) followed by high-throughput screening for mutations. Thus, plants, seeds and tissues comprising a gene having one or more of the desired mutations may be obtained using TILLING. Targeted mutagenesis is mutagenesis that may be designed to alter a specific nucleotide or nucleic acid sequence. Targeted mutagenesis may be selected from, but is not limited to, a technique selected from oligo-directed mutagenesis, RNA-guided endonucleases (e.g. the CRISPR-technology), TALENs or Zinc finger technology and combinations thereof.

Optionally, the gene of step (a) of the method of the invention is a natural sequence. The gene of step (a) of the method of the invention preferably comprises or consists of a promoter operably linked to a sequence encoding a PAR protein optionally followed by a 3′UTR sequence. The gene provided in step (a) may be part of a plant cell, preferably a plant protoplast, that does not comprise a parthenogenetic allele of a parthenogenesis gene. Preferably said plant cell or protoplast is non-parthenogenetic, i.e. a plant regenerated from said cell or protoplast does not show the parthenogenetic phenotype. Put differently, the gene provided in step (a) is preferably comprised within a plant cell, preferably a plant protoplast, wherein a mutation as defined herein results in an induced or increased parthenogenesis of the plant cell, i.e. a plant regenerated from said mutated cell or protoplast shows the parthenogenetic phenotype.

The gene provided in step (a) of the method of the invention may be a native gene preferably as naturally present in a plant cell. Native sequences are sequences found in nature and are also indicated herein as “wild type” or “natural”. Therefore, in this embodiment, the promoter, the coding sequence and the optional 3′UTR are from a single plant species.

Alternatively, the gene provided in step (a) of the method of the invention is a non-natural and/or synthetic gene also denominated herein as a chimeric gene. Optionally, in said chimeric gene, the promoter is operably linked to a coding sequence encoding a PAR protein and/or 3′UTR, wherein said coding sequence and/or 3′UTR is heterologous to the promoter. As a non-limiting example, the promoter may be of another plant species as the coding sequence and/or 3′UTR. Optionally, the promoter and 3′UTR are from a single plant species, and the coding sequence is from another plant species.

Preferably, the gene provided in step (a) of the method of the invention is present in a plant cell or protoplast. Therefore, the method of the invention may comprise a step of providing a protoplast or plant cell comprising the gene of step (a) preceding the step of modifying the promoter of the gene. Preferably, the gene is an endogenous gene that is present in the genome of the protoplast or plant cell. Preferably said protoplast or plant cell is isolated from a plant, preferably of a non-parthenogenetic plant. Preferably said plant does not have a Par allele in its genome. Preferably, the promoter of the gene provided in step (a) that is within the protoplast or plant cell, is modified as defined herein by targeted or random mutagenesis, preferably targeted mutagenesis.

In a particular embodiment of the method of the invention, the plant cell comprising the gene of step (a) may be located in a plant seed. Preferably said seed does not have a Par allele in its genome. Preferably, the promoter of the gene that is within said seed is modified in the method of the invention by targeted or random mutagenesis, preferably random mutagenesis.

After the step of modifying the promoter of the gene, the method of the invention may comprise the step of regenerating a plant from said protoplast or the step of growing a plant from said seed.

In addition, the method of the invention may comprise a step of screening and/or genotyping. Genotyping may be performed by sequencing at least part of the promoter after the modification step (optionally preceded by PCR amplification of genomic DNA and/or a target sequence comprising the promoter of interest), or by any genomic variation analysis method or molecular marker assay known in the art, such as, but not limited to, Sequence Based Genotyping (SBG) or KeyGene® SNPSelect analysis. Also “event specific” PCR diagnostic methods can be developed, where the PCR primers are based on the plant DNA flanking the modification, see U.S. Pat. No. 6,563,026. Similarly, event specific AFLP fingerprints or RFLP fingerprints may be developed which identify the transgenic plant or any plant, seed, tissue or cells derived there from. Genotyping may be performed either directly after the modification step or after growing a callus, a tissue or a plant from the protoplast or seed.

Screening for functionality in parthenogenesis may be assessed directly by comparing the ability of the plant (indicated herein as a test plant) comprising the mutant gene obtained by the method of the invention to grow and develop an embryo from an unfertilized egg cell. Preferably, this ability is compared to such ability of a control plant that preferably only differs from the test plant it does not comprise the mutant gene obtained by the method of the invention. Preferably, said control plant is a plant not comprising a Par allele.

Alternatively or in addition, functionality in parthenogenesis of the mutant gene obtained by the method of the invention can be assessed by complementation of a plant having loss-of-apomixis plant with a construct comprising the mutant gene of the invention. Such loss-of-apomixis plant may be a Taraxacum officinale isolate A68 that has been modified to lose the apomictic phenotype by modifying the functional Par allele (e.g. by deletion or knocking out). Such loss-of-apomixis plant may be a Taraxacum officinale isolate A68 that comprises a Par allele wherein SEQ ID NO: 23 (encoding PAR protein of SEQ ID NO: 32) as defined herein has been modified to any one of SEQ ID NO: 24-27 to encode the protein of SEQ ID NO: 28-31, respectively (see Table 1). Such loss of apomixis plant of Taraxacum officinale isolate A68 may be obtained by targeted genome editing using a CRISPR-Cas9/guide RNA complex, wherein said guide RNA (also indicated herein as gRNA) comprises the target specific sequence of SEQ ID NO: 19, as exemplified herein. Deletion of the Par allele of Taraxacum officinale isolate A68 results in loss-of-parthenogenesis and therefore in loss-of-apomixis. The mutant gene obtained by the method of the invention has the capability to induce parthenogenesis, and the apomictic phenotype will be restored (or rescued) upon introduction or transfection of a construct or vector comprising said mutant gene. For Taraxacum officinale isolate A68, high seed set in the absence of cross pollination is a clear indication for apomixis. Selfing in this isolate can be excluded as an alternative explanation, because due to an unbalanced triploid male and female meiosis, sexually produced egg cells and pollen grains will have a very low fertility. Preferably, in a complementation assay described above, the mutant gene of the invention is capable of restoring the apomixis in the loss-of-function plant as opposed to the original non-mutant gene. Preferably, said capability means that apomixis is restored in at least 1 out of 200, 1 out of 100, 10 out of 100, 20 out of 100, 30 out of 100, 40 out of 100, 50 out of 100, 60 out of 100, 70 out of 100, 80 out of 100, 90 out of 100 or all loss-of-function plants transformed with the mutant gene show apomixes, preferably as compared to less than 1 out of 500, 1 out of 600, 1 out of 700, 1 out of 800, 1 out of 900 or 1 out 1000 plants transformed with the original non-mutant gene (i.e. the gene provided in step a) of the method of the invention).

The invention also provides a mutant gene obtained by or obtainable by a method of the invention. Preferably, the mutant gene (only) differs from the endogenous or native gene in that it comprises an insert or deletion and/or one or more modified or removed MYB binding sites in the promoter as defined herein. Preferably, the mutant gene of the invention comprising the modified promoter as defined herein is capable of inducing the parthenogenesis phenotype to a plant. In other words, preferably a mutant gene comprising the modified promoter of the invention is functional in parthenogenesis.

Optionally, the gene of step (a) of the method of the invention and/or the mutant gene obtained by the method of the invention is, or is part of, an isolated nucleic acid molecule or nucleic acid construct or (expression) vector. The invention also provides for such isolated nucleic acid molecule, construct or (expression) vector comprising said mutant gene, wherein said construct or vector is capable of converting the mutant gene to a plant upon transfection of said plant with said construct of vector. Said nucleic acid molecule may be, but is not limited to, DNA, and may be, or may be derived of, genomic DNA. The invention also provides for the use of a mutant gene of the invention and/or an isolated nucleic acid, construct or vector comprising said mutant gene for increasing or inducing the parthenogenesis phenotype to a protoplast, plant cell or plant.

The mutant gene of the invention may be a chimeric gene as defined herein, that is optionally part of a genetic construct or nucleic acid vector. The mutant gene of the invention is optionally comprised in an isolated nucleic acid, construct or vector. In one embodiment of the invention, the nucleic acid comprising or consisting of the mutant gene of the invention may be used to make a construct, and/or a vector comprising this nucleic acid for transfer of the nucleic acid into a host cell and production of a functional (preferably capable of inducing parthenogenesis) protein encoded by said nucleic acid in host cells.

Vectors suitable for the introduction of the mutant gene of the invention in plant cells are herein referred to as i.e. “expression vectors”. Host cells are preferably plant cells. The construction of a mutant gene, construct and/or vector for, optionally transient but preferably stable, introduction of a mutant gene sequence into the genome of a host cells is generally known in the art.

The invention also provides a plant cell, plant protoplast, plant tissue, seed or plant comprising mutant gene obtained by or obtainable by the method of the invention, and/or comprising the nucleic acid molecule or vector.

The invention also provides a plant cell, plant protoplast, plant tissue, seed or plant comprising the mutant gene, nucleic acid molecule, construct or vector comprising the mutant gene as defined herein. Optionally, said plant cell, plant protoplast, plant tissue, seed or plant is capable of apomeiosis, preferably apomixis.

Preferably, a nucleic acid of the invention is an isolated nucleic acid. In an embodiment, the nucleic acid of the invention may originate from Taraxacum lines (e.g. Taraxacum officinale sensu lato) or from other plant species. In one embodiment, the nucleic acid of the invention is from a different origin than from Taraxacum or Taraxacum officinale sensu lato.

Optionally, the nucleic acid or nucleic acid construct of the invention comprising or consisting of the mutant gene obtained by or obtainable by the method of the invention, can be stably inserted into the nuclear genome of a single plant cell, and the so-transformed plant cell can be used to produce a transformed plant that has an altered phenotype, i.e. a parthenogenetic phenotype. In a non-limiting example, a T-DNA vector, comprising the mutant gene as taught herein, in Agrobacterium tumefaciens can be used to transform the plant cell, and thereafter, a transformed plant can be regenerated from the transformed plant cell using the procedures described, for example, in EP0116718, EP0270822, PCT publication WO84/02913 and published European Patent application EP0242246 and in Gould et al. (1991). The construction of a T-DNA vector for Agrobacterium mediated plant transformation is well known in the art. The T-DNA vector may be either a binary vector as described in EP0120561 and EP0120515 or a co-integrate vector which can integrate into the Agrobacterium Ti-plasmid by homologous recombination, as described in EP0116718. Lettuce transformation protocols have been described in, for example, Michelmore et al. (1987) and Chupeau et al. (1989).

Border sequences are described in Gielen et al. (1984). Of course, other types of vectors can be used to transform the plant cell, using procedures such as direct gene transfer (as described, for example in EP0223247), pollen mediated transformation (as described, for example in EP0270356 and WO85/01856), protoplast transformation as, for example, described in U.S. Pat. No. 4,684,611, plant RNA virus-mediated transformation (as described, for example in EP0067553 and U.S. Pat. No. 4,407,956), liposome-mediated transformation (as described, for example in U.S. Pat. No. 4,536,475), and other methods.

In a further embodiment, the mutant gene of the invention may be introduced by somatic hybridization. Somatic hybridization may be done by protoplast fusion (e.g. see Holmes, 2018).

The mutant gene of the invention can also be integrated in the genome for instance by using one or more specific endonucleases (such as a CRISPR-endonuclease/guide RNA complex) for introducing double strand breaks at the appropriate site in the genome and a donor construct comprising the mutant gene of the invention for integration in the genome. The skilled person knows how to design such CRISPR-endonuclease/guide RNA complex for introducing a double strand break and donor construct suitable for integration (for a review, see Bortesi and Fischer, 2015).

Likewise, selection and regeneration of transformed plants from transformed cells is well known in the art. Obviously, for different species and even for different varieties or cultivars of a single species, protocols are specifically adapted for regenerating transformants at high frequency. The invention also encompasses progeny of the transformed plants showing parthenogenesis and comprising the mutant gene of the invention.

Besides transformation of the nuclear genome, also transformation of the plastid genome, preferably chloroplast genome, is included in the invention. One advantage of plastid genome transformation is that the risk of spread of the transgene(s) can be reduced. Plastid genome transformation can be carried out as known in the art, see e.g. Sidorov et al. (1999) or Lutz et al. (2004).

The resulting transformed plant can be used in a conventional plant breeding scheme to produce more transformed plants containing the mutant gene. Single copy transformants can be selected, using e.g. Southern Blot analysis or PCR based methods or the Invader® Technology assay (Third Wave Technologies, Inc.). Transformed cells and plants can easily be distinguished from non-transformed ones by the presence of the mutant gene of the invention, characterized in comprising the modified promoter as defined herein. The sequences of the plant DNA flanking the insertion site of the mutant gene can also be sequenced, whereby an “Event specific” detection method can be developed, for routine use. See for example WO0141558, which describes elite event detection kits (such as PCR detection kits) based for example on the integrated sequence and the flanking (genomic) sequence.

In one embodiment, the invention encompasses a mutant gene that is derived from a par allele originating from a plant that is non-parthenogenetic (and non-apomictic) by nature, and is modified by the method of the invention. Such plant may be a wild or cultivated plant. Said mutant gene is preferably obtained by modification of the promoter by the method of the invention, and is characterized in that it comprises an insert or deletion in the promoter and/or one or more modified or removed MYB binding sites in the promoter as defined herein.

In one embodiment, the mutant gene of the invention, or nucleic acid, vector or construct comprising said mutant gene, has a (genetically) dominant function, preferably provided by (over)expressing a functional protein having the amino acid sequence SEQ ID NO: 1, or a variant or functional fragment thereof, such as an orthologue or fragment thereof found in another plant (i.e. other than Taraxacum or Taraxacum officinale sensu lato).

Preferably, the mutant gene of the invention, or nucleic acid, vector or construct comprising said mutant gene, encodes a protein or functional fragment(s) thereof which, when produced in the plant, is functional and induces and/or enhances parthenogenesis.

Preferably, the modified promoter, mutant gene, nucleic acid, vector and/or construct do not occur naturally, i.e. do not occur in nature.

The gene provided in step (a) of the method of the invention may be an endogenous gene present in the genome of a plant cell or protoplast. Preferably, said plant cell or protoplast is part of or is isolated from a plant (indicated herein as the originating plant) not having the parthenogenesis phenotype. The plant cell or protoplast comprising the mutant gene obtained by the method of the invention may be part of or regenerate into a plant having the parthenogenesis phenotype or showing a significant increase in parthenogenesis phenotype as compared to the originating plant. Therefore, the present invention also provides a method for converting the parthenogenesis phenotype to a plant or a method for increasing the parthenogenesis phenotype to a plant. In other words, the method of the invention provides for producing a parthenogenetic plant.

Therefore, the invention provides a method for producing a parthenogenetic plant, comprising the steps of:
(A) regenerating and/or growing a plant tissue or a plant from the plant cell or protoplast comprising the mutant gene obtainable by a method for producing a mutant gene as defined herein; and (B) optionally, screening and/or genotyping the plant tissue or plant obtained in step (A).

Hence, the present invention also provides a method for producing a parthenogenetic plant, wherein said method comprises the steps of:
a) providing one or more plants, plant protoplasts, plant cells, plant tissues or plant seeds comprising a gene encoding a PAR protein operably linked to a promoter, wherein the promoter preferably comprises one or more transcription factor MYB binding sites;
b) modifying the promoter by modifying at least one of the one or more transcription factor MYB binding sites and/or the sequence upstream of the one or more transcription factor MYB binding sites to increase expression of the encoded PAR protein;
c) optionally, growing one or more plants from the modified plant protoplasts, plant cells, plant tissues or seeds obtained in step b); and
d) optionally, screening and/or genotyping the plant protoplasts, plant cells, plant tissues or seeds obtained in step b) or the plant obtained in step b) or c).

Alternatively or in addition, the present invention provides a method for producing a parthenogenetic plant, wherein said method comprises the steps of:
a) providing one or more plants, plant protoplasts, plant cells, plant tissues or plant seeds comprising a gene encoding a PAR protein operably linked to a promoter optionally comprising one or more transcription factor MYB binding sites;
(b) modifying the promoter to increase expression of the encoded PAR protein, preferably by at least one of:
   i) introducing an insert in the promoter sequence upstream of the start codon of the sequence encoding a PAR protein as defined herein, preferably upstream or directly upstream of the one or more transcription factor MYB binding sites, wherein the insert preferably introduces an enhancer sequence or removes a repressor sequence;
   ii) introducing a substitution and/or deletion the promoter sequence upstream of the start codon of the sequence encoding a PAR protein as defined herein, preferably upstream or directly upstream of the one or more transcription factor MYB binding sites, wherein the substitution and/or deletion preferably introduces an enhancer sequence or removes a repressor sequence; and
   (iii) a combination of (i) and (ii).
c) optionally, growing one or more plants from the modified plant protoplasts, plant cells, plant tissues or seeds obtained in step b); and
d) optionally, screening and/or genotyping the plant protoplasts, plant cells, plant tissues or seeds obtained in step b) or the plant obtained in step b) or c).

The parthenogenetic plant produced by a method of the invention may be a plant having a normal meiotic function, i.e. not showing apomeiosis, preferably not being diplosporous and/or not showing diplospory. Preferably the gametophyte of said plant may have a reduced ploidy as compared to its somatic cells. In case of a diploid plant (i.e. having diploid somatic cells), said reduced ploidy may be mono-haploid. Upon induction of parthenogenesis to a plant via the method of the present invention, a gametophyte of said plant may develop in a plant with reduced ploidy, preferably a haploid plant. Hence a method of the invention may be a method of producing a plant with a reduced ploidy, preferably a method of producing a haploid plant. The method preferably comprises the steps of providing a parthenogenetic plant obtainable by a method as defined above, and a subsequent step of allowing said plant to produce seed without fertilization, allowing one or more of said seed to germinate and regenerate into a plant of reduced ploidy, preferably a haploid plant. Said method may comprise the steps of inducing parthenogenesis as defined herein, followed by allowing said plant to produce seed without fertilization, and allowing one or more of said seeds to germinate and regenerate into a plant of reduced ploidy, preferably into a haploid plant (a plant with haploid somatic cells).

A genome with reduced ploidy, preferably a haploid genome, may be doubled spontaneously or induced, preferably by chemical treatment. A preferred chemical treatment is e.g. described in Touchell D H et al, Front Plant Sci. 2020 Jun. 3; 11:722, which is incorporated herein by reference. The chemical treatment may be a treatment with at least one of colchicine, oryzalin, trifluralin and nitrous oxide. Chemical treatment of a plant preferably results in a plant with a doubled haploid genome.

A doubled mono-haploid plant is a plant that has achieved homozygosity at all loci and that can be obtained by whole genome duplication of a mono-haploid genome, preferably using a method as described herein. Such fully homozygous plants are amongst others commercially important for use as parent plants in the production of F1 hybrid seeds. Hence, the invention also provides for a method of producing doubled mono-haploid plants comprising the steps of producing a parthenogenetic plant as defined herein, optionally comprising the step of chemically inducing genome duplication, selecting doubled mono-haploid seed, and optionally allowing said seed to germinate and regenerate into a doubled haploid plant.

The method of the invention is not limited to the production of a doubled mono-haploid plant. The method as described herein is equally suitable for the production other doubled haploid plants, such as, but not limited to, doubled di-haploid, doubled tri-haploid, doubled tetra-haploid, doubled penta-haploid and doubled hexa-haploid plants.

As a non-limiting example, in polyploid crops (e.g. a tetraploid Solanum tuberosum), the method of the invention can be used for the production of di-haploid offspring. The degree of heterozygosity of these di-haploids will be much lower than that of the polyploid parents. Selection at the di-haploid level will therefore be much more efficient than at the tetraploid level. The method of the invention can further be used to generate mono-haploids from these di-haploids that are completely homozygous. Di-haploids may facilitate the construction of genetic maps and enable the assembly of phased whole-genome sequences. Valuable traits of interest of a wild diploid species may be introgressed through the use of di-haploids from cultivars produced by the method of the invention. The obtained di-haploids with valuable introgressed traits can be made doubled di-haploid e.g. using a chemical treatment as described herein above, thereby allowing the introgression of the trait of interest into the tetraploid cultivars. The skilled person readily understands that a similar method may be used for the production of a diploid, triploid, pentaploid, hexaploid, heptaploid, septaploid etc. cultivars having an introgressed trait of interest of a wild species.

Preferably, the gene encoding the PAR protein in step a) is a parallele. Preferably, the promoter of the gene is modified in step b) as defined herein, thereby converting a par allele into a Par allele. As the Par allele may be dominant, altering a single par allele into a Par allele of a plant or plant cell, or introducing a mutant gene of the invention in a plant or plant cell for instance by transfection of said plant or plant cell with a vector comprising said mutant gene, can be sufficient to convert a plant from a sexual phenotype to a parthenogenesis phenotype, i.e. resulting in a plant and/or offspring thereof that is capable of growing and developing an embryo from an egg cell without fertilization. Therefore, preferably a single sexual gene, preferably an endogenous gene, present in a plant cell is modified by the method of the invention. Optionally, multiple genes, preferably endogenous genes, present in a plant cell are modified by the method of the invention.

In case the modified promoter is present in a plant cell or protoplast, said plant cell or protoplast may be part of or regenerate into a plant that is thereby converted from having a non-parthenogenesis phenotype into a plant having the parthenogenesis phenotype. Therefore, the present invention also provides a method for conferring the parthenogenesis phenotype to a plant not showing the parthenogenesis phenotype by modifying the promoter of a par allele. In other words, the method of the invention is a method to transform a plant not showing parthenogenesis into a plant showing parthenogenesis. Preferably, the one or more plants, plant protoplasts, plant cells, plant tissues or plant seeds of step a) lack a Par allele and/or or do not show the parthenogenetic phenotype. Similarly, the method of the invention is a method to transform a plant showing limited parthenogenesis into a plant having increased parthenogenesis. The increase or induction of parthenogenesis preferably means that at least 1 out of 200, 1 out of 100, 10 out of 100, 20 out of 100, 30 out of 100, 40 out of 100, 50 out of 100, 60 out of 100, 70 out of 100, 80 out of 100, 90 out of 100 or all plants transformed with the mutant gene of the invention show parthenogenesis, preferably as compared to less than 1 out of 500, 1 out of 600, 1 out of 700, 1 out of 800, 1 out of 900 or 1 out 1000 plants transformed with the original non-mutant gene (i.e. the gene provided in step a) of the method of the invention).

Preferably, the gene of step a) of the method of the invention is a native gene. Optionally, the gene to be modified by the method of the invention is a par allele of Taraxacum officinale or any one of the orthologous parthenogenesis genes as defined herein. Preferably said parallele is present in a plant, plant, plant protoplast, plant cell, plant tissue or plant seed that lacks a Par allele and is therefore does not have the parthenogenesis phenotype. By conferring said par allele into a Par allele, the method of the invention preferably results in the transformation of a plant not having the parthogenesis phenotype into a plant having the parthenogenesis phenotype.

Optionally multiple plants, plant protoplasts, plant cells, plant tissues or plant seeds are provided in step a) and after step b) one or more plants, plant protoplasts, plant cells, plant tissues or plant seeds are selected that comprise the modification of the invention, i.e. the insert or deletion and/or modified or removed one or more MYB binding sites, preferably determined by genotyping and/or screening as defined herein. Screening in step B) may thus be screening for the parthenogenesis phenotype.

Apart from genetically modifying a naturally occurring par allele in a plant cell (that may be part of a plant tissue, plant seed or whole plant) or plant protoplast to confer said par allele to a Par allele, and growing and/or developing said cell or protoplast into a parthenogenetic plant, a parthenogenetic plant may also be obtained by transforming a plant or plant cell with a nucleic acid, construct or vector comprising the mutant gene of the invention, i.e. that comprises the modified promoter operably linked to a coding sequence encoding a PAR protein as defined herein, optionally linked to a 3′UTR sequence.

The mutant gene obtained by the method of the invention can be introduced in one or more plant cells by transforming, introgression, somatic hybridization and/or protoplast fusion. Such mutant gene may be located on an exogenous nucleic acid, i.e. a nucleic acid not occurring in said plant cell in nature.

Therefore, the present invention also provides a method for producing a parthenogenetic plant, wherein said method comprises the steps of:
a. providing one or more plants, plant protoplasts, plant cells, plant tissues or plant seeds;
b. transforming the one or more plants, plant protoplasts, plant cells, plant tissues or plant seeds with a nucleic acid construct comprising the mutant gene of the present invention;
c. optionally, growing one or more plants from the plant protoplasts, plant cells, plant tissues or seeds transformed in step b); and
d. optionally, screening and/or genotyping the plant protoplasts, plant cells, plant tissues or seeds obtained in step b) or the plant obtained in step b) or c).

Preferably, the one or more plants of step a. lack a Par allele and/or or do not show the parthenogenetic phenotype.

In a further aspect, the present invention relates to plants (including e.g. plant cells, organs, seeds and plant parts), as obtained by any of the methods defined above. Preferably, these are parthenogenetic plants or show increased parthenogenesis as compared to a native or unmodified plant.

Preferably, the plant of the invention is obtained by a technical means, preferably by a method as described herein. Such technical means are well-known to the skilled person and include genetic modifications, such as e.g. at least one of random mutagenesis, targeted mutagenesis and nucleic acid insertions or deletions.

Preferably, the plant of the invention is not obtained by an essentially biological process. Preferably, the plant of the invention is not exclusively obtained by an essentially biological process. Preferably, the plant of the invention is not obtained, preferably not directly obtained, by any essentially biological process that introduces parthenogenesis in a plant. Preferably, the plant of the invention is not exclusively obtained by any essentially biological process that introduces parthenogenesis in a plant. Preferably, the plant of the invention is not a naturally occurring plant, i.e. is not a plant that occurs in nature.

In an embodiment, the promoter of the gene of the method of the invention is an upstream transcription regulatory region, e.g. within about 2000 bp upstream of the translation start codon and/or transcription start site of the gene and can be isolated from apomictic plants and/or other plants using known methods, such as TAIL-PCR (Liu et al., 1995; Liu et al., 2005), Linker-PCR, or Inverse PCR (IPCR). A chimeric gene as defined herein may be produced by linking the promoter to a coding sequence as taught herein, preferably having the amino acid sequence of SEQ ID NO: 1, or functional variant and/or fragment thereof, which is optionally subsequently linked upstream (i.e. 5′) of a suitable 3′ end non-translated region (“3′end” or 3′UTR). Suitable 3′ends include those of the CaMV 35S gene (“3′ 35S”), the nopaline synthase gene (“3′ nos”) (Depicker et al., 1982), the octopine synthase gene (“3′ocs”) (Gielen et al., 1984) and the T-DNA gene 7 (“3′ gene 7”) (Velten and Schell, 1985), which act as 3′-untranslated DNA sequences in transformed plant cells, and others. In one embodiment, a 3′UTR of a native parthenogenesis gene is used, or a 3′UTR derived therefrom. For example, any 3′UTR derived from SEQ ID NO: 4, or a variant or fragment thereof, may be used. The 3′UTR may have the nucleotide sequence of SEQ ID NO: 4.

Introduction of the T-DNA vector into Agrobacterium can be carried out using known methods, such as electroporation or triparental mating.

A mutant gene as taught herein, can optionally be inserted in the plant genome as a hybrid gene sequence linked in-frame to a (U.S. Pat. No. 5,254,799; Vaeck et al., 1987) gene encoding a selectable or scorable marker, such as for example the neo (or nptII) gene (EP0242236) encoding kanamycin resistance, so that the plant that comprises the nucleic acid is easily detectable.

Optionally, the mutant gene of the invention can be changed to modify possibly (further) transcription factor binding sites, preferably to modify binding sites for transcription factors that repress gene transcription.

In one embodiment, the PAR protein encoded by the nucleic acid of the invention as taught herein is co-expressed with other proteins which control, preferably enhance or induce, parthenogenesis, apomeiosis or apomixis in a single host, optionally under control of different promoters. Such other gene may be the gene for conferring apomeiosis, such as diplospory e.g. as described in WO2017/039452 A1, which is incorporated herein by reference.

In another embodiment, the mutant gene of the invention is introgressed in germplasm that preferably comprises other genes of interest, such as the gene for conferring apomeiosis (e.g. the gene for diplospory). Via crossing and selection, hybrids are produced wherein several genes of interest may be stacked.

Optionally, the plant mutated or transformed by the method of the invention is a plant that is capable of apomeiosis. Preferably a plant capable of apomeiosis is modified to comprise a mutant gene functional in parthenogenesis as defined herein. Such mutation or modification will then result in an apomictic plant or plant cell. In that case, the screening in step B) of the method of producing a parthenogenetic plant as defined herein may be for the apomictic phenotype. A plant cell capable of apomeiosis may be obtained by introduction a nucleic acid capable of conferring apomeiosis. Optionally said nucleic acid is introduced in a plant cell before, together or after the introduction of a mutant gene of the present invention.

The invention also provides a method of producing an apomictic hybrid seed, comprising the steps of:
(1) cross-fertilizing a sexually reproducing first plant with the pollen of a second plant to produce F1 hybrid seeds; and
(2) optionally selecting from the said F1 seeds a seed that comprise the apomictic phenotype;
wherein said first and/or second plant is capable of apomeiosis and wherein said second plant is a parthenogenetic plant obtained or obtainable by the method of the invention, and wherein preferably said selecting is performed by genotyping. Optionally, said method further comprises the step of selecting from the said F1, seeds that comprise the apomictic phenotype, preferably by genotyping, and optionally growing at least one F1 plant from said F1 hybrid seed.

Preferably, for selection purposes but also for weed control options, the transgenic plants of the invention are also transformed with a DNA encoding a protein conferring resistance to herbicide, such as a broad-spectrum herbicide, for example herbicides based on glufosinate ammonium as active ingredient (e.g. Liberty® or BASTA; resistance is conferred by the PAT or bar gene; see EP 0 242 236 and EP 0 242 246) or glyphosate (e.g. RoundUp®; resistance is conferred by EPSPS genes, see e.g. EPO 508 909 and EP 0 507 698). Using herbicide resistance genes (or other genes conferring a desired phenotype) as selectable marker further has the advantage that the introduction of antibiotic resistance genes can be avoided.

Alternatively or in addition, other selectable marker genes may be used, such as antibiotic resistance genes. As it is generally not accepted to retain antibiotic resistance genes in the transformed host plants, these genes can be removed again following selection of the transformants. Different technologies exist for removal of transgenes. One method to achieve removal is by flanking the transgene with lox sites and, following selection, crossing the transformed plant with a CRE recombinase-expressing plant (see e.g. EP506763B1). Site specific recombination results in excision of the marker gene. Another site specific recombination system is the FLP/FRT system described in EP686191 and U.S. Pat. No. 5,527,695. Site specific recombination systems such as CRE/LOX and FLP/FRT may also be used for gene stacking purposes. Further, one-component excision systems have been described, see e.g. WO9737012 or WO9500555).

Preferably, the mutant gene of the invention is used to generate transgenic plant cells, plants, plant seeds, etc. and any derivatives/progeny thereof, with an enhanced parthenogenetic phenotype. Preferably, the transgenic plants of the invention comprise enhanced parthenogenesis compared to the unmodified control plant. Thus, for example transgenic lettuce plants comprise enhanced parthenogenesis are provided. Thus, a plant comprising a mutant gene of the invention shows a significant increase in parthenogenesis, as compared to the same plant not comprising said mutant gene. The enhanced parthenogenesis phenotype can be fine-tuned by expressing a suitable amount of the protein encoded by the mutant gene of the invention capable of inducing parthenogenesis at a suitable time and/or location. Such fine-tuning may be done by determining the most appropriate promoter modification and/or by selecting transgenic “events” which show the desired expression level.

Transformants, hybrids or inbreds expressing desired levels of the protein encoded by the mutant gene of the invention are selected by e.g. analysing copy number (Southern blot analysis), mRNA transcript levels (e.g. RT-PCR using primer pairs capable of amplifying the protein encoded by the mutant gene of the invention or flanking primers) or by analysing the presence and level of parthenogenesis protein in various tissues (e.g. SDS-PAGE; ELISA assays, etc). Single copy transformants may be selected, for instance for regulatory reasons, and the sequences flanking the site of insertion of the mutant gene is analysed, preferably sequenced to characterize the “event”. Transgenic events resulting in high or moderate expression of the protein encoded by the mutant gene of the invention are selected for further development until a high performing elite event with a stable transgene is obtained.

Transformants comprising a mutant gene of the invention, may also comprise (other) transgenes, such as genes conferring disease resistance or conferring tolerance to other biotic and/or abiotic stresses, or conferring diplospory. To obtain such plants with “stacked” transgenes, other transgenes may either be introduced into said transformants, or said transformants may be transformed subsequently with one or more other genes, or alternatively several chimeric genes may be used to transform a plant line or variety. For example, several transgenes may be present on a single vector, or may be present on different vectors which are co-transformed.

In one embodiment the following genes are combined with the mutant gene of the invention: known disease resistance genes, especially genes conferring enhanced resistance to necrotrophic pathogens, virus resistance genes, insect resistance genes, abiotic stress resistance genes (e.g. drought tolerance, salt tolerance, heat- or cold tolerance, etc.), herbicide resistance genes, and the like. The stacked transformants may thus have an even broader biotic and/or abiotic stress tolerance, to pathogen resistance, insect resistance, nematode resistance, salinity, cold stress, heat stress, water stress, etc.

Whole plants, plant parts (e.g. seeds, cells, tissues), and plant products (e.g. fruits) and progeny of any of the plants described herein are encompassed herein and can be identified by the presence of the mutant gene, for example by PCR analysis using total genomic DNA as template and using PCR primer pairs specific for the mutant gene of the invention and/or by using genomic variation analysis such as, but not limited to, Sequence Based Genotyping (SBG) or KeyGene® SNPSelect analysis. Also “event specific” PCR diagnostic methods can be developed, where the PCR primers are based on the plant DNA flanking the inserted modification or transgene, see U.S. Pat. No. 6,563,026. Similarly, event specific AFLP fingerprints or RFLP fingerprints may be developed which identify the transgenic or mutant plant of the invention, or any plant, seed, tissue or cells derived there from.

It is understood that the transgenic or mutant plants according to the invention preferably do not show non-desired phenotypes, such as yield reduction, enhanced susceptibility to diseases (especially to necrotrophs) or undesired architectural changes (dwarfing, deformations) etc. and that, if such phenotypes are seen in the primary transformants, these can be removed by conventional methods. Any of the transgenic or mutant plants described herein may be heterozygous, homozygous or hemizygous for the mutant gene.

The invention also pertains to a plant, seed, plant part (e.g. a plant cell) and plant product obtained or obtainable by the method as detailed herein, preferably comprising the mutant gene of the invention, the nucleic acid of the invention and/or the construct of the invention. Preferably said mutant gene, nucleic acid and/or construct is capable of inducing parthenogenesis and/or functional in parthenogenesis, as detailed herein. The plant of the invention preferably is of a species listed herein as suitable host plant. Such method to obtain a plant of the invention include, but is not limited to, random or targeted mutagenesis, introgression of the mutant gene of the invention from a plant into progeny, and/or transformation of plant cells by a mutant gene of the invention, and subsequent regeneration of a plant from said plant cell.

Preferably the plant, plant part and/or plant product is not of the species Taraxacum officinale sensu lato, comprising a mutant gene of the invention. Preferably, the plant, plant part and/or plant product is an eudicot. Said plant or plant cell preferably is of a species listed herein as suitable host plant, preferably from the family selected from the group consisting of Brassicaceae, Cucurbitaceae, Fabaceae, Gramineae, Solanaceae and Asteraceae (Compositae).

Preferably the plant, plant part and/or plant product comprising the mutant gene of the invention is obtained by genetic modification or by introgression, wherein preferably said mutant gene is located in its genome. Preferably said plant, plant part and/or plant product is capable of parthenogenesis and/or shows parthenogenesis. Even more preferably said plant, plant part and/or plant product is further capable of apomeiosis. The invention provides seed, plant parts or plant products of a plant or plant cell of the invention.

The invention also pertains to plant parts and plant products derived from the plant of the invention, wherein the plant parts and/or plant products comprise the mutant gene of the invention as defined herein, the nucleic acid of the invention as defined herein and/or the construct of the invention as defined herein, which may be fragments as defined herein that allow for assessing the presence of such protein, mutant gene, nucleic acid or construct in the plant from which the plant part of plant product is derived. Such parts and/or products may be seed or fruit and/or products derived therefrom (e.g. sugars or protein). Such parts, products and/or products derived therefrom may be non-propagating material.

Any plant may be a suitable host, but most preferably the host plant species should be a plant species which would benefit from enhanced parthenogenesis. Suitable hosts include any plant species. Particularly, cultivars or breeding lines having otherwise good agronomic characteristics are preferred. The skilled person knows how to test whether the mutant gene as taught herein, and/or variants or fragments thereof, can confer the required increase or reduction of parthenogenesis onto the host plant, by generating transgenic plants and assessing parthenogenesis, together with suitable control plants.

Suitable host plants include for example hosts which belong to the Brassicaceae, Cucurbitaceae, Fabaceae, Gramineae, Solanaceae, Asteraceae (Compositae), Rosaceae or Poaceae.

In a preferred embodiment, the host plant may be a plant species selected from the group consisting of the genera Taraxacum, Lactuca, Pisum, Capsicum, Solanum, Cucumis, Zea, Gossypium, Glycine, Tryticum, Oryza and Sorghum.

In a preferred embodiment, the plant, plant part, plant cell or seed as taught herein is from a species selected from the group consisting of the genera Taraxacum, Lactuca, Pisum, Capsicum, Solanum, Cucumis, Zea, Gossypium, Glycine, Triticum, Oryza, Allium, Brassica, Helianthus, Beta, Cichorium, Chrysanthemum, Pennisetum, Secale, Hordeum, Medicago, Phaseolus, Rosa, Lilium, Coffea, Linum, Cannabis, Cassava, Daucus, Cucurbita, Citrullus, and Sorghum.

Suitable host plants include for example maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), safflower, yam, cassava, alfalfa (Medicago sativa), rice (Oryza species, e.g. O. sativa indica cultivar-group or japonica cultivar-group), forage grasses, pearl millet (Pennisetum spp. e.g. P. glaucum), tree species (Pinus, poplar, fir, plantain, etc), tea, coffea, oil palm, coconut, vegetable species, such as pea, zucchini, beans (e.g. Phaseolus species), hot pepper, cucumber, artichoke, asparagus, eggplant, broccoli, garlic, leek, lettuce, onion, radish, turnip, tomato, potato, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Gerbera species), herbs (mint, parsley, basil, thyme, etc.), woody trees (e.g. species of Populus, Salix, Quercus, Eucalyptus), fibre species e.g. flax (Linum usitatissimum) and hemp (Cannabis sativa).

A mutant gene obtained or obtainable by the method of the invention or a nucleic acid of the invention comprising said mutant gene may be used for conferring parthenogenesis, for conferring apomixis for increasing ploidy and/or for producing a double haploid. Preferably said use is in plant biotechnology and/or breeding, i.e. in/on plant or plant cells.

Parthenogenesis is an element of apomixis and a gene for parthenogenesis could be used in combination with a gene for apomeiosis (e.g. diplospory) to generate apomixis, preferably to use it for the applications listed herein. These genes can be introduced into sexual crops by transformation, introgression or by modifying endogenous suitable genes thereby converting them in apomeiotic (or diplosporous) genes. Knowledge of the structure and function of the apomixis genes can also be used to modify endogenous sexual reproduction genes in such a way that they become apomixis genes. The preferred use would be to bring the apomixis genes under a inducible promoter such that apomixis can be switched off when sexual reproduction generates new genotypes and switched on when apomixis is needed to propagate the elite genotypes.

The mutant gene of the invention can be used as a component of apomixis. Both apomeiosis and parthenogenesis are required for functional gametophytic apomixis. Apomeiosis can be achieved by a combination of mutations affecting meiosis (Crismani et al., 2013), with the outcome of chromosomal non-reduction in megaspores, i.e., mitosis rather than meiosis. Somatic cells that assume a gametophytic fate through epigenetic alterations (Grimanelli, 2012) also result in unreduced spore-like cells that potentially can give rise to unreduced gametes (egg cells). In another embodiment, apomeiosis is achieved by transgenic or non-transgenic expression of a natural apomeiosis gene. By whatever means unreduced egg cells are formed, proper temporal and spatial expression from the mutant gene of the invention can induce the egg cells to behave as zygotes and divide in the absence of fertilization.

A mutant gene of the invention could be used in entirely new ways, e.g. not directly as tool in apomixis. For example whereas in apomixis both parthenogenesis and apomeiosis are combined in a single plant, the use of apomeiosis in one generation and the use of parthenogenesis in the next generation would link sexual gene pools of a crop at the diploid and at the polyploid level, by going up in ploidy level by apomeiosis and going down in ploidy level by parthenogenesis. This is very useful because polyploid populations may be better for mutation induction because they can tolerate more mutations. Polyploid plants can also be more vigorous. However diploid populations are better for selection and diploid crosses are better for genetic mapping, the construction of BAC libraries etc. Parthenogenesis in polyploids may generate haploids which can be crossed with diploids. Diplospory in diploids generates unreduced 2n egg cells which can be fertilized by pollen from polyploids to produce polyploid offspring. Thus, an alternation of apomeiosis and parthenogenesis in different breeding generations links the diploid and the polyploid gene pools.

Another use of the mutant gene of the invention without apomeiosis, is the production of haploid offspring, which could be used for the production of haploids and by genome doubling of doubled haploids (DHs) (e.g. spontaneous genome doubling, colchicine, sodium azide or other chemicals). Doubled haploids can be used as parents to produce sexual F1 hybrids. Doubled haploids is the fastest methods to make plants homozygous. With doubled haploids plants can be made homozygous, whereas with the second fastest method, selfing, it takes 5-7 generations to reach a sufficiently high level of homozygosity in diploid plants. There are several methods to produce doubled haploids. In some plant species haploids can be generated by microspore culture. Other methods are the production of haploid embryos (gynogenesis) by pollination with irradiated pollen (melon), or the pollination with specific pollinator stocks (maize, potato). These methods have their limitations, such as costs, recalcitrance of genotypes, labour intensity etc. In some crops no methods for haploid production exist (e.g. tomato). With the dominant allele of the parthenogenesis gene the frequency of gynogenesis could be significantly increased, reducing the costs of haploid production.

The following non-limiting Examples illustrate the different embodiments of the invention. Unless stated otherwise in the Examples, all recombinant DNA techniques are carried out according to standard protocols as described in Sambrook et al. (1989), and Sambrook and Russell (2001); and in Volumes 1 and 2 of Ausubel et al. (1994). Standard materials and methods for plant molecular work are described in Plant Molecular Biology Labfax (1993) by R. D. D. Croy, jointly published by BIOS Scientific Publications Ltd (UK) and Blackwell Scientific Publications, UK.

TABLE 1
Overview of SEQ ID NOs used herein.

| SEQ ID NO | Name |
|---|---|
| 1 | Par allele protein Taraxacum officinale |
| 2 | Par allele promoter Taraxacum officinale |
| 3 | Par allele coding sequence Taraxacum officinale |
| 4 | Par allele 3′UTR Taraxacum officinale |
| 5 | Par allele gene Taraxacum officinale |
| 6 | par allele-1 protein Taraxacum officinale |
| 7 | par allele-1 promoter Taraxacum officinale |
| 8 | par allele-1 coding sequence Taraxacum officinale |
| 9 | par allele-1 3′UTR gene Taraxacum officinale |
| 10 | par allele-1 gene Taraxacum officinale |
| 11 | par allele-2 protein Taraxacum officinale |
| 12 | par allele-2 promoter Taraxacum officinale |
| 13 | par allele-2 coding sequence Taraxacum officinale |
| 14 | par allele-2 3′UTR gene Taraxacum officinale |
| 15 | par allele-2 gene Taraxacum officinale |
| 16 | Lactuca sativa LSAT_8X112340 promoter |
| 17 | Lactuca sativa LSAT_8X112340 promoter without 2 MYB binding sites |
| 18 | Lactuca sativa LSAT_8X112340 promoter with Taraxacum officinale Par allele |
| | MITE insertion |
| 19 | gene specific part of guide RNA-1 for Par allele |
| 20 | Lactuca sativa LSAT_8X112340 promoter with deletion |
| 21 | Helianthus annuus_XR_002563155.1 |
| 22 | Lactuca sativa _PLY80414.1 |
| 23 | nucleotides 325-360 of the Par allele (wild type) |
| 24 | mutated sequence of nucleotides 325-360 of the Par allele (1 bp insertion) |
| 25 | mutated sequence of nucleotides 325-360 of the Par allele (1 bp insertion) |
| 26 | mutated sequence of nucleotides 325-360 of the Par allele (1 bp deletion) |
| 27 | mutated sequence of nucleotides 325-360 of the Par allele (3 bp deletion) |
| 28 | encoded amino acid sequence of SEQ ID NO: 24 |
| 29 | encoded amino acid sequence of SEQ ID NO: 25 |
| 30 | encoded amino acid sequence of SEQ ID NO: 26 |
| 31 | encoded amino acid sequence of SEQ ID NO: 27 |
| 32 | encoded amino acid sequence of SEQ ID NO: 23 |
| 33 | Lactuca sativa LSAT_8X112340 coding sequence |
| 34 | LsPromoter_LsCoding sequence_To3′UTR |
| 35 | LsPromoter-without-MYB-sites_LsCoding sequence_To3′UTR |
| 36 | LsPromoter-with-MITE-insert_LsCoding sequence_To3′UTR |
| 37 | CXXCXXXXXXX[K/R]AXXGHX[R/N]XH K2-2 zinc finger domain |
| 38 | CXXCXXXXXXX[X]XXXGHXRXH zinc finger domain consensus sequence |
| 39 | Cichorium endivia PAR protein |
| 40 | Hieracium praealtum of aurantiacum PAR protein |
| 41 | Senecio cambrensis PAR protein |
| 42 | Hevea brasiliensis PAR protein |
| 43 | Cucurbita moschata PAR protein |
| 44 | Eutrema salsugineum PAR protein |
| 45 | Arachis ipaensis PAR protein |
| 46 | Cajanus cajan PAR protein |
| 47 | Brassica_rapa PAR protein |
| 48 | Lagenaria siceraria PAR protein |
| 49 | Arachis ipaensis PAR protein |
| 50 | Prunus_persica PAR protein |
| 51 | Glycine max PAR protein |
| 52 | Glycine max PAR protein |
| 53 | Glycine max PAR protein |
| 54 | Glycine max PAR protein |
| 55 | Cicer arietinum_fabales PAR protein |
| 56 | Cicer arietinum PAR protein |
| 57 | Cicer arietinum PAR protein |
| 58 | EAR motif |
| 59 | EAR motif |
| 60 | Tar-MITE insert |
| 61 | LsPromoter-with-deletion_LsCoding sequence_To3′UTR |
| 62 | Arabidopsis thaliana DUO1 |
| 63 | LsPromoter_ToCoding sequence_To3′UTR |
| 64 | LsPromoter-with-MITE-insert_ToCoding sequence_To3′UTR |
| 65 | ToPromoter_LsCoding sequence_To3′UTR |

### LEGENDS TO THE FIGURES

FIGS. 1A-1G. Complementation and transformation experiments in the Taraxacum CRISPR/Cas9 Loss of Parthenogenesis mutant and sexual lettuce. FIG. 1A) Different promoter-gene constructs used for complementation of the Taraxacum LOP mutant and the number of successfully complemented lines. Shown are the ToPar promoter and the sexual homolog from lettuce (Lspar) and the Arabidopsis egg cell (EC1.1) promoter with the Taraxacum Pargene. FIG. 1B) Similar for transformation of lettuce with the Taraxacum ToPar gene driven by Arabidopsis egg cell EC1.1 promoter). FIG. 1C), FIG. 1D), FIG. 1E), Embryo-like structures in decapitated inflorescences of lettuce transformed with a pEC1.1::Par construct. FIG. 1C) Embryo sac from control non-transformed lettuce 75 h after decapitation. Unfertilized egg cell (ec) and central cell (cc) nuclei are visible. FIG. 1D) Embryo sac with developing embryo-like structures 75 h after decapitation. FIG. 1E) Embryo sac with multiple embryo-like structures. Asterisks show individual embryo-like structures. FIG. 1F) Flow cytometry analysis of embryo sacs from control non-transformed lettuce five days after self-pollination. FIG. 1G), Flow cytometry analysis of embryo sacs from transgenic lettuce carrying pEC1. 1::Par construct five days after decapitation.

FIG. 2. Polymorphisms in the Taraxacum Par/par promoters. ClustalW alignment of the region 350 bp upstream of the ATG start codon (underlined) of the Parallele and three sexual alleles: par-1 and par-2 of Taraxacum officinale (Topar) and of the sexual allele of the rubber dandelion Taraxacum koksaghyz (Tkpar). The 1335 bp MITE insertion has been removed from the ToPar promoter. The 6 bp direct repeat, which is the insertion site of the MITE, is underlined. Of the 13 SNPs, three are between the PAR promoter the sexual promoters (in bold and underlined); ten occur between the sexual promoters.

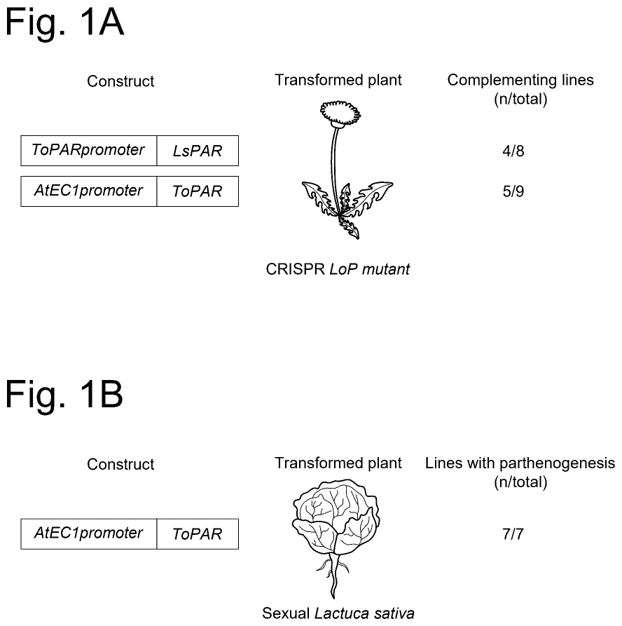

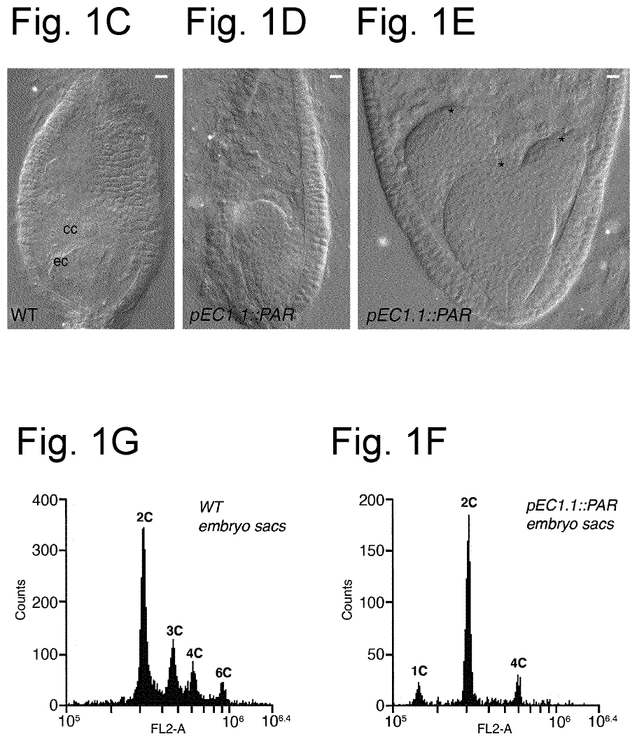

### EXAMPLES

### Example 1

### Induction of Parthenogenesis by the Par Promoter

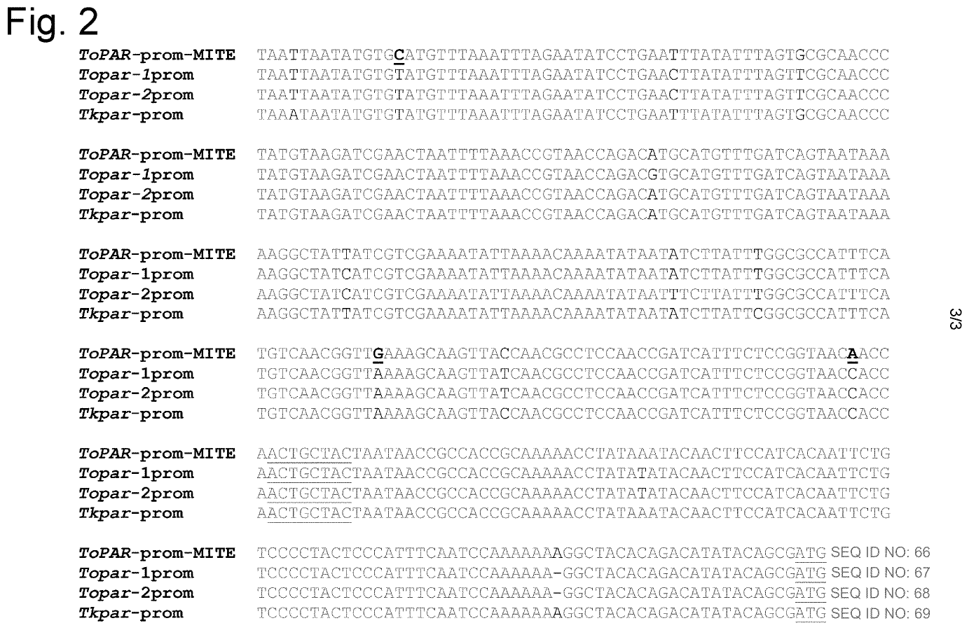

To test if the Taraxacum Par promoter plays a role in the genetic control of parthenogenesis, we tested whether it could be combined with a Par coding sequence homologue from a sexual species to induce parthenogenesis. The Taraxacum Par promoter was used to drive the expression of a homologous gene (Lspar) from lettuce, a related Asteraceae species and an important vegetable crop plant. This construct was transformed into a self-incompatible, tetraploid Taraxacum CRISPR/Cas9 Loss of Parthenogenesis (LOP) mutant that was derived from a cross between the 3×PAR CRISPR mutant derived from the A68 line, with pollen from diploid plant FCH72, and that cannot produce viable seeds. As the Parallele is dominant, testing was performed on the primary transformed plants (TO). Remarkably the Par::Lspar construct led to seed formation and tetraploid (due to presence of the dominant Diplospory gene) offspring in four independent transformants (Table 2). This demonstrates that the Taraxacum Par promoter can invoke a lettuce gene to induce parthenogenesis. No genetic polymorphisms specific to the ToPar coding sequence (when compared to the sexual alleles par and par2) were found in the lettuce gene, ruling out coding sequence polymorphisms as being causal for parthenogenesis. Moving upstream from the Par ATG start site, the MITE insertion represents the first genetic polymorphism that is unique to the apomictic allele when compared to three sexual alleles from dandelion (par1, par2 and par^{TKS}; FIG. 2). Only 13 SNPs are found between the four Taraxacum promoters in the 350 bp upstream of the ATG (when the MITE is excised from the PAR allele) of which only three are Par-allele specific. Taken together this provides strong evidence that the functional difference between the dandelion alleles of the Par gene is caused by the promoter and not by the coding sequence. As previously demonstrated, a different construct, where the Par gene is expressed under the egg cell specific Arabidopsis EC1 (pEC1::Par) promoter, can also lead to complementation of the CRISPR/Cas9 LOP mutant, consistent with the hypothesis that egg cell expression of PAR can cause parthenogenesis (see Example 2 of PCT/EP2020/064991 and Table 3).

This experiment proves that the Taraxacum Par promoter driving expression of a sexual lettuce gene rescues the parthenogenetic phenotype in a loss-of-parthenogenesis Taraxacum plant. In other words, the Taraxacum Par promoter driving expression of a sexual lettuce gene is capable of inducing parthenogenesis.

TABLE 2
Apomixis complementation of the T. officinale
4x CRISPR/Cas9 PAR deletion mutant with the ToPar
promoter fused with the lettuce Lspar gene.
Primary transformants were grown in the greenhouse, seeds collected
and germination tests of up to three seed-heads (SH; 30 seeds per
seed-head) performed. All progeny plants tested contained the PCR
marker for the dominant DIP gene indicating recombination and
reduction did not occur in female meiosis. Ploidy level of progenies
was measured by flow cytometry (FCM). Seedlings produced by
parthenogenesis are tetraploid; hexaploid (6x) seedlings result
from self-fertilization (a tetraploid diplosporous egg cell
fertilized by a reduced diploid pollen grain). Four of the eight
lines produced parthenogenetic tetraploid offspring. Although
T. officinale is self-incompatible it is known that the SI
system can be leaky (Morita et al. 1990; and Tas and Van Dijk,
1999). Occasionally hexaploid progeny were observed from a control
plant transformed with a 35S::GUS construct, indicating that self-
fertilization can (rarely) occur in this genetic background. This
can explain the single hexaploid offspring of the non-
complementing line yellow 12b.

| | | | | | | FCM | | | DIP |
|---|---|---|---|---|---|---|---|---|---|
| plant | SH1 | SH2 | SH3 | # Seedlings | | analyzed | 4x | 6x | positive |
| 1 | − | − | − | 0 | (90) | | | | |
| 2 | − | + | − | 0 | (90) | | | | |
| 3 | − | + | | 7 | (60) | 3 | 3 | | 3 |
| 4 | + | + | + | 15 | (90) | 9 | 9 | | 9 |
| 5 | − | − | + | 6 | (90) | 1 | | 1 | 1 |
| 6 | + | + | + | 12 | (90) | 4 | 4 | | 4 |
| 7 | + | + | | 15 | (60) | 8 | 8 | | 8 |
| 8 | − | − | − | 0 | (90) | | | | |
| Total | | | | 55 | (660) | 25 | 24 | 1 | 25 |

(8.3%)

TABLE 3
Apomixis complementation of the T. officinale 4x
CRISPR/Cas9 PAR deletion mutant by the ToPAR gene under
the Arabidopsis EC1.1 promoter. See Table 2 for an explanation.
Among the plants transformed with the complementation construct
one plant produced four 6x seedlings due to selfing.

| | | | | | | FCM | | | DIP |
|---|---|---|---|---|---|---|---|---|---|
| plant | SH1 | SH2 | SH3 | # Seedlings | | analyzed | 4x | 6x | positive |
| 1 | + | − | − | 4 | (90) | 4 | 4 | | 4 |
| 5 | + | − | − | 7 | (90) | | | | |
| 3 | + | | | 3 | (30) | 2 | 2 | | 2 |
| 4 | − | − | − | 0 | (90) | | | | |
| 5 | + | + | + | 11 | (90) | 4 | | 4 | 4 |
| 6 | + | − | − | 5 | (90) | 3 | 3 | | 3 |
| 7 | + | | | 5 | (30) | 3 | 3 | | 3 |
| 8 | − | − | − | 0 | (90) | | | | |
| 9 | + | | | 10 | (30) | 3 | 3 | | 3 |
| Total | | | | 45 | (630) | 19 | 15 | 4 | 19 |

(7.1%)

### Example 2

### Plant Material

For this experiment, wild type lettuce: Iceberg type, Legacy, Takii Japan and Red Romaine type, Baker Creek Heirloom Seeds was used.

### DNA Construct

A binary vector is constructed with a T-DNA region comprising the construct represented by SEQ ID NO: 34 consisting of the following consecutive elements: a LSAT_8X112340 promoter of Lactuca sativa (SEQ ID NO: 16) driving expression of the LSAT_8X112340 CDS sequence of Lactuca sativa (SEQ ID NO: 33), followed by the first 1000 bases of the 3′UTR of the Par allele gene of Taraxacum officinale (the first 1000 bases of SEQ ID NO: 4), followed by a 35S terminator and a neomycin phosphotransferase gene (nptII) for selection. Suitable technologies to generate such a binary vector are Gateway®, Golden Gate or Gibson Assembly® (for an example, see Ma et al., 2015). Transgenic lines harbouring this T-DNA are numbered with the code pKG20001.

A second binary vector is constructed with a T-DNA region comprising the construct represented by SEQ ID NO: 35 consisting of the following consecutive elements: a LSAT_8X112340 promoter of Lactuca sativa amended to remove two MYB binding sites having the sequences AACCGCCA and AACCGTC (SEQ ID NO: 17) driving expression of the LSAT_8X112340 CDS sequence of Lactuca sativa (SEQ ID NO: 33), followed by the first 1000 bases of the 3′UTR of the Par allele gene of Taraxacum officinale (the first 1000 bases of SEQ ID NO: 4), followed by a 35S terminator and a neomycin phosphotransferase gene (nptII) for selection. Suitable technologies to generate such a binary vector are Gateway®, Golden Gate or Gibson Assembly® (for an example, see Ma et al., 2015). Transgenic lines harbouring this T-DNA are numbered with the code pKG20002.

A third binary vector is constructed with a T-DNA region comprising the construct represented by SEQ ID NO: 36 that consists of the following consecutive elements: a LSAT_8X112340 promoter of Lactuca sativa with an insertion of the Par allele gene of Taraxacum officinale MITE promoter element (SEQ ID NO: 18) driving expression of the LSAT_8X112340 CDS sequence of Lactuca sativa (SEQ ID NO: 33) followed by the first 1000 bases of the 3′UTR of the Par allele gene of Taraxacum officinale (the first 1000 bases of SEQ ID NO: 4), followed by a 35S terminator and a neomycin phosphotransferase gene (nptII) for selection. Suitable technologies to generate such a binary vector are Gateway®, Golden Gate or Gibson Assembly® (for an example, see Ma et al., 2015). Transgenic lines harbouring this T-DNA are numbered with the code pKG20003.

A fourth binary vector is constructed with a T-DNA region comprising the construct represented by SEQ ID NO: 61 that consists of the following consecutive elements: a LSAT_8X112340 promoter of Lactuca sativa with a deletion upstream of the two MYB binding sites (SEQ ID NO: 20) driving expression of the LSAT_8X112340 CDS sequence of Lactuca sativa (SEQ ID NO: 33) followed by the first 1000 bases of the 3′UTR of the Par allele gene of Taraxacum officinale (the first 1000 bases of SEQ ID NO: 4), followed by a 35S terminator and a neomycin phosphotransferase gene (nptII) for selection. Suitable technologies to generate such a binary vector are Gateway®, Golden Gate or Gibson Assembly® (for an example, see Ma et al., 2015). Transgenic lines harbouring this T-DNA are numbered with the code pKG20003.

### Plant Transformation Method

Agrobacterium transformation is performed by genotype-independent transformation of lettuce using Agrobacterium tumefaciens. Such methods are well-known in the art and e.g. taught in Curtis et al. (1994). Any other method suitable for genetic transformation of lettuce may be used to produce plants harbouring the desired T-DNA, such as described in Michelmore et al. (1987) or Chupeau et al. (1989).

### Results

Plants that are positively tested for presence of the transgene as described under section “DNA construct” above, are evaluated for occurrence of parthenogenesis. As the trait is dominant, testing is performed on the primary transformed plants (TO). In the absence of cross or self-fertilization, parthenogenetic egg cells develop into embryos. In order to prevent any fertilization of the plants harboring the transgene, plants are grown in a greenhouse and prior to microscopic observation, all flowers are manually emasculated. Emasculation is performed by clipping the involucre before the corolla has grown. Parthenogenesis is detected in non-apomictic plants microscopically by Nomarski Differential Interference Microscopy (DIC) of cleared ovules. Here, the clearing method using chloral hydrate is applied; a method commonly used to clear ovules of plants for microscopic imaging (see e.g. Franks RG, 2016). At 75 hours post emasculation, flower buds are harvested and ovules are cleared with chloral hydrate. In transgenic lines of pKG20002 and pKG20003 multiple embryos may be observed in these cleared ovules. Flow cytometry on pools of embryo sacs can show that these embryos are haploid. In non-transformed control plants and in evaluated transgenic lines of pKG20001, which are emasculated and imaged in the same way, no embryos were observed at all.

These results will demonstrate that either inserting the MITE promoter element from the Par allele gene of Taraxacum officinale or removing MYB binding sites from the LSAT_8X112340 promoter of Lactuca sativa is sufficient to modify the expression as such that the LSAT_8X112340 gene can induce haploid embryo formation in lettuce.

### Example 3

### Plant Material

For this experiment, wild type lettuce: Red Romaine type, Baker Creek Heirloom Seeds was used.

### DNA Construct

A binary vector was constructed with a T-DNA region comprising the construct represented by SEQ ID NO: 63 consisting of the following consecutive elements: a LSAT_8X112340 promoter of Lactuca sativa (SEQ ID NO: 16) driving expression of the Par CDS sequence of Taraxacum officinale (SEQ ID NO: 3), followed by the first 1000 bases of the 3′UTR of the Par allele gene of Taraxacum officinale (the first 1000 bases of SEQ ID NO: 4), followed by a 35S terminator and a neomycin phosphotransferase gene (nptII) for selection. Suitable technologies to generate such a binary vector are Gateway®, Golden Gate or Gibson Assembly® (for an example, see Ma et al., 2015). Transgenic lines harbouring this T-DNA are numbered with the code pKG20004.

A second binary vector was constructed with a T-DNA region comprising the construct represented by SEQ ID NO: 64 consisting of the following consecutive elements: a LSAT_8X112340 promoter of Lactuca sativa with an insertion of the Parallele gene of Taraxacum officinale MITE promoter element (SEQ ID NO: 18) driving expression of the Par CDS sequence of Taraxacum officinale (SEQ ID NO: 3) followed by the first 1000 bases of the 3′UTR of the Par allele gene of Taraxacum officinale (the first 1000 bases of SEQ ID NO: 4), followed by a 35S terminator and a neomycin phosphotransferase gene (nptII) for selection. Suitable technologies to generate such a binary vector are Gateway®, Golden Gate or Gibson Assembly® (for an example, see Ma et al., 2015). Transgenic lines harbouring this T-DNA are numbered with the code pKG20006.

A third vector was constructed with a T-DNA region comprising the construct represented by SEQ ID NO: 65 consisting of the following consecutive elements: a Par allele promoter of Taraxacum officinale (SEQ ID NO: 2) driving expression of the LSAT_8X112340 CDS sequence of Lactuca sativa (SEQ ID NO: 33), followed by the first 1000 bases of the 3′UTR of the Par allele gene of Taraxacum officinale (the first 1000 bases of SEQ ID NO: 4), followed by a 35S terminator and a neomycin phosphotransferase gene (nptII) for selection. Suitable technologies to generate such a binary vector are Gateway®, Golden Gate or Gibson Assembly® (for an example, see Ma et al., 2015). Transgenic lines harbouring this T-DNA are numbered with the code pKG20008.

### Plant Transformation Method

Agrobacterium transformation is performed by genotype-independent transformation of lettuce using Agrobacterium tumefaciens. Such methods are well-known in the art and e.g. taught in Curtis et al. (1994). Any other method suitable for genetic transformation of lettuce may be used to produce plants harbouring the desired T-DNA, such as described in Michelmore et al. (1987) or Chupeau et al. (1989).

### Results

Plants that positively tested for presence of the transgene as described under section “DNA construct” above, were evaluated for occurrence of parthenogenesis. As the trait is dominant, testing was performed on the primary transformed plants (TO). In the absence of cross or self-fertilization, parthenogenetic egg cells develop into embryos. In order to prevent any fertilization of the plants harboring the transgene, plants were grown in a greenhouse and prior to microscopic observation, all flowers were manually emasculated. Emasculation was performed by clipping the involucre before the corolla has grown. Parthenogenesis was detected in non-apomictic plants microscopically by Nomarski Differential Interference Microscopy (DIC) of cleared ovules. Here, the clearing method using chloral hydrate was applied; a method commonly used to clear ovules of plants for microscopic imaging (see e.g. Franks R G. 2016). At 75 hours post emasculation, flower buds were harvested and ovules are cleared with chloral hydrate. In transgenic lines of pKG20005, pKG20006, pKG20007 and pKG20008 multiple embryos were observed in these cleared ovules (see table 3). Flow cytometry on pools of embryo sacs can show that these embryos are haploid. In standard GUS construct transformed control plants and in evaluated transgenic lines of pKG20004, which were emasculated and imaged in the same way, no embryos were observed at all.

These results demonstrated that a MITE promoter element from the Par allele gene of Taraxacum officinale is sufficient to modify the expression, such that the LSAT_8X112340 gene can induce haploid embryo formation in lettuce. This is a clear example of inducing parthenogenesis in lettuce by promoter modifications of the lettuce LSAT_8X112340 gene as in the absence of cross or self-fertilization, egg cells developed into embryos.

TABLE 3
embryo observations in flower buds at 75 hours post emasculation
of lettuce transgenic lines. The indication “yes”
means that in all buds there is at least one embryo.

| | Phenotyping | |
|---|---|---|
| | number of | embryos found |
| Construct-Plant ID | buds checked | in all buds |
| GUS construct control 1 | 9 | no |
| GUS construct control 2 | 4 | no |
| GUS construct control 3 | 11 | no |
| GUS construct control 4 | 8 | no |
| GUS construct control 5 | 6 | no |
| GUS construct control 6 | 7 | no |
| GUS construct control 7 | 7 | no |
| pKG20004-1 | 6 | no |
| pKG20004-2 | 11 | no |
| pKG20004-3 | 9 | no |
| pKG20004-4 | 14 | no |
| pKG20004-5 | 6 | no |
| pKG20004-6 | 7 | no |
| pKG20004-7 | 8 | no |
| pKG20004-8 | 8 | no |
| pKG20004-9 | 4 | no |
| pKG20006-1 | 4 | yes |
| pKG20006-2 | 18 | yes |
| pKG20006-3 | 14 | yes |
| pKG20008-1 | 16 | yes |
| pKG20008-2 | 9 | no |
| pKG20008-3 | 8 | yes |
| pKG20008-4 | 8 | no |
| pKG20008-5 | 7 | no |

### REFERENCES

An et al. (1996) Plant J. 10, 107
Asker, S. (1979) Progress in apomixis research. Hereditas 91(2): 231-240.
Asker, S. E. and Jerling, L. (1990) Apomixis in Plants. CRC Press, Boca Raton.
Ausubel et al. (1994) Current Protocols in Molecular Biology, Volumes 1 and 2, Current Protocols, USA
Barrell and Grossniklaus (2005) Confocal microscopy of whole ovules for analysis of reproductive development: the elongatel mutant affects meiosis II. Plant Journal 34: 309-320.
Bicknell and Koltunow 2004 Understanding apomixis: recent advances and remaining conundrums. The Plant Cell 16: S228-S245.
Borevitz, J. O., Liang, D., Plouffe, D., Chang, H.-S., Zhu, T., Weigel, D., Berry, C. C., Winzeler, E. and Chory, J. (2003) Large-scale identification of single-feature polymorphisms in Arabidopsis. Genome Res. 13: 513-523.
Bortesi, L. and Fischer, R. (2015) The CRISPR/Cas9 system for plant genome editing and beyond. Biotechnology Advanced 33(1): 41-52.
Borg et al. (2011) The R2R3 MYB Transcription Factor DUO1 Activates a Male Germline-Specific Regulon Essential for Sperm Cell Differentiation in Arabidopsis. The Plant Cell 23 (2): 534-549
Chupeau et al. (1989) Transgenic plants of lettuce (Lactuca sativa) obtained through electroporation of protoplasts. Bio/Technology 7, 503-508.
Crismani W. et al. (2013) J. Exp. Bot. 64:55-65.
Curtis I S et al. (1994) J. Exp. Bot. 45.10: 1441-1449.
Daniell, H. (2002) Molecular strategies for gene containment in transgenic crops. Nature biotechnology 20: 581-586.
Depicker A. and Van Montagu M. (1997) Post-transcriptional gene silencing in plants. Current Opinion in Cell Biology 9: 373-382.
Depicker et al. (1982) J. Mol. Appl. Genetics 1, 561-573.
Englbrecht et al. (2004) BMC Genomics, 5 (1): 39
Franks R G (2016) Methods Mol Biol. 2016; 1457:1-7
Gielen et al. (1984) EMBO J 3, 835-845.
Guo et al, Scientific reports. 2017 Jun. 1; 7(1):2634.
Gould et al. (1991) Plant Physiol. 95, 426-434.
Grimanelli D. (2012) Curr. Opin. Plant Biol. 15:57-62.
Hashimshony, T., Senderovich, N., Avital, G. et al. CEL-Seq2: sensitive highly-multiplexed single-cell RNA-Seq. Genome Biol 17, 77 (2016).
Hashimshony T, Wagner F, Sher N, Yanai I. CEL-Seq: single-cell RNA-Seq by multiplexed linear amplification. Cell Rep. 2012; 2(3):666-673.
Henikoff and Henikoff (1992) PNAS 89, 915-919.
Hermsen, J. G. Th. (1980) Breeding for apomixis in potato: Pursuing a utopian scheme. Euphytica 29:595-607.
Holmes, M (2018) Historical Studies in the Natural Sciences, 48 (1). pp. 1-23. ISSN 1939-1811
Hull and Howell (1987) Virology 86, 482-493.
Kagale et al., (2010) Plant Physiology, 152: 1009-1134.
Liu et al. (1995) Genomics 25(3):674-81.
Liu et al. (2005) Methods Mol. Biol. 286:341-8.
Lutz K A et al. (2004) Plant J. 37(6):906-13.
Ma, Xingliang, et al. “A robust CRISPR/Cas9 system for convenient, high-efficiency multiplex genome editing in monocot and dicot plants.” Molecular plant 8.8 (2015): 1274-1284.
McPherson at al. (2000) PCR-Basics: From Background to Bench, First Edition, Springer Verlag, Germany.
Michelmore, R. W., Marsh, E., Seely, S. and Landry, B. (1987) Transformation of lettuce (Lactuca sativa) mediated by Agrobacterium tumefaciens. Plant Cell Rep. 6: 439-442.
Michelmore, R. W., Paran, I. and Kesseli, R. V. (1991) Identification of markers linked to disease resistance genes by bulked segregant analysis: a rapid method to detect markers in specific genomic regions using segregating populations. Proc. Natl. Acad. Sci. 88:9828-9832.
Morgan, R., Ozias-Akins, P., and Hanna, W. W. (1998) Seed set in an apomictic BC3 pearl millet. Int. J. Plant Sci. 159, 89-97.
Morita, T. Menken, S. G. J. and Sterk, A. A. (1990) Hybridization between European and Asian dandelions (Taraxacum section Ruderalia and section Mongolica): 1. Crossability and breakdown of self-incompatibility. New Phytol. 114, 519-529.
Ozias-Akins, P. and P. J. van Dijk. (2007) Mendelian genetics of apomixis in plants. Annu. Rev. Genet. 41:509-537.
Park et al. (1997) J. Biol. Chem. 272, 6876-6881.
Plant Molecular Biology Labfax (1993) by R. D. D. Croy, jointly published by BIOS Scientific Publications Ltd (UK) and Blackwell Scientific Publications, UK.
Sambrook and Russell (2001) Molecular Cloning: A Laboratory Manual, Third Edition, Cold Spring Harbor Laboratory Press, NY.
Sambrook et al. (1989) Molecular Cloning: A Laboratory Manual, Second Edition, Cold Spring Harbor Laboratory Press.
Savidan Y. (2001) Transfer of apomixis through wide crosses. In: Savidan Y, Carman J, Dresselhaus T, editors. The flowering of apomixis: From mechanisms to genetic engineering. Mexico: CIMMYT, IR D; pp. 153-167.
Sidorov V A et al. (1999) Plant J. 19: 209-216.
Smith T F, Waterman M S (1981) J. Mol. Biol 147(1); 195-7.
Tas I. C. Q. and Van Dijk, P. J. (1999) Heredity (Edinb). 83, 707-714
Vaeck et al. (1987) Nature 328, 33-37.
Van Baarlen, De Jong, J. H., and Van Dijk, P. J. (2002) Comparative cyto-embryological investigations of sexual and apomictic dandelions (Taraxacum) and their apomictic hybrids. Sex Plant Reprod 15: 31-38.
Van Dijk, P. J., Rigola, D. and Schauer, S. E. “Plant breeding: surprisingly, less sex is better.” Current Biology 26.3 (2016): R122-R124.
Velten and Schell (1985) Nucleic Acids Research 13, 6981-6998.
Vielle-Calzada, J-Ph., B. L. Burson, E. C Bashaw, and M. A. Hussey 1995. Early fertilization events in the sexual an aposporous egg apparatus of Pennisetum ciliare (L.) Link, The Plant Journal 8(2):309-316.
Yang, J. et al. (2018) PlantEAR: Functional Analysis Platform for Plant EAR Motif-Containing Proteins. Front. Genet.

1.-15. (canceled)

16. A method of producing a mutant gene capable of inducing a parthenogenesis phenotype, wherein said method comprises the steps of:
(a) providing a gene comprising a sequence encoding a PAR protein of SEQ ID NO: 1, 6 or 11 or any orthologue thereof, operably linked to a promoter comprising one or more transcription factor MYB binding sites, wherein the PAR protein is characterized in that it comprises a zinc finger C2H2-type domain having the consensus amino acid sequence of SEQ ID NO: 37 and an EAR motif having the consensus amino acid sequence of SEQ ID NO: 58 or 59; and
(b) modifying the promoter by modifying the sequence upstream of the one or more MYB binding sites to increase transcription of the encoded PAR protein.

17. The method according to claim 16, wherein the modifying comprises introducing an insert or deletion into the promoter directly upstream of the one or more MYB binding sites.

18. The method according to claim 16, wherein the promoter is modified by random or targeted mutagenesis.

19. The method according to claim 16, wherein the gene is comprised within a plant cell.

20. The method according to claim 19, wherein the plant cell is a plant protoplast.

21. The method according to claim 19, wherein the mutant gene induces or increases parthenogenesis in the plant cell.

22. The method according to claim 16, wherein the gene is an endogenous gene.

23. The method according to claim 16, wherein the gene is a transgene that is optionally a native or a chimeric gene.

24. A mutant gene capable of inducing the parthenogenesis phenotype comprising a sequence encoding a PAR protein of SEQ ID NO: 1, 6 or 11, or any orthologue thereof, wherein the mutant gene comprises a modification as defined in claim 16.

25. A nucleic acid molecule, construct or vector comprising the mutant gene of claim 24.

26. A plant cell comprising the mutant gene of claim 24.

27. The plant cell according to claim 26, which is a plant-protoplast.

28. A plant comprising the mutant gene of claim 22, wherein the plant is a parthenogenetic plant.

29. A plant obtainable by producing a mutant gene as defined in claim 16.

30. The plant according to claim 29, wherein the plant is capable of apomeiosis.

31. The plant according to claim 30, which is apomictic.

32. A method for producing a parthenogenetic plant, comprising:
(a) regenerating and/or growing a plant tissue or a plant from the plant cell of claim 26; and optionally,
(b) screening and/or genotyping the plant tissue or plant obtained in (a).

33. The method according to claim 32, wherein the mutant gene of (a) is comprised in a plant cell capable of apomeiosis.

34. The method according to claim 33, wherein the plant cell is a plant protoplast.

35. A method for producing an apomictic F1 hybrid seed, comprising:
(i) cross-fertilizing a sexually reproducing first plant with the pollen of a second plant to produce F1 hybrid seeds, wherein the second plant comprises the mutant gene of claim 22, and wherein the first and/or second plant is capable of apomeiosis; and optionally
(ii) selecting from the F1 hybrid seed, seeds that comprise the apomictic phenotype.
